(19) United States
(12) Patent Application Publication
(10) Pub. No.: US 2022/0090060 A1
(43) Pub. Date: Mar. 24, 2022

(54) IDENTIFICATION AND PRODUCTION OF ANTIGEN-SPECIFIC ANTIBODIES

(71) Applicant: Regeneron Pharmaceuticals, Inc., Tarrytown, NY (US)

(72) Inventors: Yashu Liu, Burlingame, CA (US); Chunguang Guo, Briarcliff Manor, NY (US); Qian Zhang, Lexington, MA (US); Natasha Levenkova, Fair Lawn, NJ (US); Andrew J. Murphy, Croton-on-Hudson, NY (US); William Olson, Yorktown Heights, NY (US)

(21) Appl. No.: 17/472,132

(22) Filed: Sep. 10, 2021

Related U.S. Application Data
(60) Provisional application No. 63/077,133, filed on Sep. 11, 2020.
(60) Provisional application No. 63/077,140, filed on Sep. 11, 2020.

(51) Int. Cl. C12N 15/10

(52) U.S. Cl. CPC C12N 15/1072

(57) ABSTRACT

A method of obtaining a nucleotide sequence, from an immunized genetically modified non-human mammal, encoding an immunoglobulin variable domain of an antibody specific for a particular antigen is disclosed. A method for making antibodies against a particular antigen is also disclosed.

### CROSS REFERENCE TO RELATED APPLICATIONS

This application claims the benefit of U.S. Provisional Patent Application No. 63/077,133, filed Sep. 11, 2020 and U.S. Provisional Patent Application No. 63/077,140, filed Sep. 11, 2020, the contents of both of which are incorporated herein by reference in their entirety.

### SEQUENCE LISTING

In accordance with 37 C.F.R. § 1.52(e)(5), a Sequence Listing in the form of an ASCII text file (entitled “Sequence_Listing”, created on Nov. 8, 2021 and 12 KB in size) is incorporated herein by reference in its entirety.

### FIELD OF THE INVENTION

Methods for obtaining nucleic acids encoding antibody amino acid sequences, such as variable domain amino acid sequences, which are specific for an antigen are provided. Methods are disclosed that include obtaining, from an immunized host, nucleic acid sequences encoding antibody sequences from a first sample, and a plurality of antibodies from a second sample that are directed against the antigen of interest, in order to obtain nucleotide sequences encoding a human immunoglobulin variable domain specific for the antigen or portion thereof. Methods of making antibodies directed against an antigen of interest are also disclosed.

### BACKGROUND

Antibodies typically comprise a heavy chain component, wherein each heavy chain monomer is associated with a light chain, with the variable domains of these chains combining to form an antigen-binding site. Antibodies, particularly monoclonal antibodies, have a wide range of uses in diagnostics and therapeutics.

Two traditional approaches have been used for monoclonal antibody preparation: hybridoma technology and DNA display (e.g., in phage, yeast or bacterial systems). In hybridoma technology, B cells from immunized animals are typically fused with myeloma cell lines to produce antigen-secreting hybridoma lines. Cells producing monoclonal antibodies of interest are isolated, grown in culture, and the resultant desired antibodies purified. High-quality purification is critical in order to remove contaminants. Thus, isolation of antibodies through hybridoma technology is not efficient because of throughput limitations of hybridoma culture.

Display technology involves production of a lead antibody candidate from a phage, yeast or mammalian library. Though direct DNA isolation from B cells expressing antibodies may be utilized, DNA libraries are expressed in cell expression systems, such as phage, yeast, or bacterial systems, then “panned” or titrated to select for the antibodies having high affinities. Display technologies can provide high-quality protein libraries, although they provide limited diversity. Consequently, in vitro mutagenesis-based affinity maturation is frequently a next step in generating high affinity antibodies derived from such libraries.

Further, antibodies are often expressed and isolated from plasma, serum, ascites fluid, cell culture medium, and bacterial cultures. These are all sources containing numerous contaminants. Therefore, efficient purification of antibodies from such sources is necessary. Thus, there remains a need in the art for efficient generation and isolation of antibodies with a requisite specificity and binding affinity for a target antigen.

### SUMMARY

The current disclosure describes, among other things, methods for obtaining antibodies using a combination of mass spectrometry (“MS”) and next generation sequencing (“NGS”). Also disclosed are methods for making antibodies.

Provided methods enable efficient identification and/or selection of sequences of human immunoglobulin variable domains and/or complementarity-determining region (CDR) sequences of antibodies, and in particular, antibodies from a host (e.g., a genetically modified non-human animal, e.g., a rodent) that has been immunized with an antigen of interest. In some embodiments, provided methods include a step of comparing and/or interrogating a plurality of antibody sequences of a host (e.g., a library of antibody sequences generated by NGS) with and/or against an MS analysis of antibody peptides from of the host. A “database” as used herein can be an exemplary “library.”

In some embodiments, provided methods comprise obtaining and/or producing a plurality of immunoglobulin variable domain and/or CDR sequences (e.g., a library) from a host immunized with an antigen of interest (e.g., from B cells of a non-human animal, e.g., a rodent). In some embodiments, a library of antibody sequences comprises a plurality of nucleic acid sequences obtained by NGS. In some embodiments, a library of antibody sequences comprises a plurality of CDR3 sequences.

In some embodiments, provided methods include MS analysis of a sample of antibodies obtained from a host (e.g., rodent) that has been immunized with an antigen of interest. The present disclosure encompasses a recognition that a sample of antibodies for MS analysis can be enriched for desired characteristics in vivo and/or ex vivo. For example, a sample of antibodies may be enriched based on in vivo localization. Accordingly, in some embodiments a sample of antibodies can be obtained from any desired source within the host, e.g., serum, plasma, lymphoid organs, gut, cerebrospinal fluid, brain, spinal cord, placenta, or a combination thereof. In some embodiments, a sample of antibodies may be enriched ex vivo for one or more desired characteristics (e.g., antigen binding, binding to a cell, etc.). The present disclosure provides the insight that such enrichment in combination with provided methods enables identification of antibodies that may be difficult to identify by other methods (e.g., because present at low titer). In some embodiments, the disclosure provides a method of obtaining a human immunoglobulin variable domain or a complementarity-determining region (CDR) of an antibody specific for an antigen. In some embodiments, a method described herein comprises interrogating amino acid sequences of a plurality of human immunoglobulin variable domains from a first sample with peptide sequences of heavy and/or light chain variable domains of a population of antibodies from a second sample. In some instances, performing an interrogation step thereby obtains a human immunoglobulin variable domain or a CDR sequence of an antibody specific for the antigen. In some embodiments, interrogation comprises aligning peptide sequences of heavy and/or light chain variable domains of the population of antibodies to each other and to amino acid sequences of the plurality of immunoglobulin variable domains.

In some embodiments, a human immunoglobulin variable domain or a CDR (e.g., CDR3) of an antibody specific for an antigen is obtained from a host immunized with a particular antigen. In some embodiments, a host is a genetically modified non-human mammal. In some embodiments, a host comprises in its genome, such as its germline genome, an immunoglobulin heavy chain variable region comprising one or more human heavy chain V gene segments (also referred to as human V_{H} gene segments), one or more human D gene segments (also referred to as human D_{H} gene segments), and one or more human heavy chain J gene segments (also referred to as human J_{H} gene segments). In some embodiments, a heavy chain variable region is operably linked to a constant region (e.g., an immunoglobulin heavy chain constant region).

In some embodiments, a host comprises in its genome, such as its germline genome, an immunoglobulin light chain variable region comprising one or more human light chain V gene segments (also referred to has human V_{L} gene segments) and one or more human light chain J gene segments (also referred to has human J_{L} gene segments). In some embodiments, a light chain is operably linked to a constant region (e.g., an immunoglobulin light chain constant region).

In some embodiments, a method described herein comprises obtaining, from a first sample from an immunized host, a plurality of nucleic acids encoding a plurality of human immunoglobulin variable domains and determining amino acid sequences of the encoded plurality of immunoglobulin variable domains. In some embodiments, a method described herein comprises obtaining, from the immunized host, a second sample comprising a population of antibodies directed against the antigen and determining therefrom peptide sequences of heavy and/or light chain variable domains of the population of antibodies.

In some embodiments, a host is a rodent such as a rat or a mouse.

In some embodiments, the disclosure provides a method of identifying a human immunoglobulin variable domain or CDR sequence (e.g., CDR3 sequence) of an antibody specific for an antigen, comprising: (i) obtaining or determining a plurality of peptide sequences of human immunoglobulin heavy chain and/or light chain variable domains that were obtained from a sample comprising a population of antibodies produced by a rodent immunized with the antigen, and (ii) interrogating a library of human immunoglobulin heavy chain and/or light chain variable domain sequences with the plurality of peptide sequences determined by MS, wherein the library comprises a plurality of human immunoglobulin heavy chain and/or light chain variable domain sequences encoded by B cells of the immunized rodent, thereby obtaining a human immunoglobulin variable domain or CDR sequence of an antibody specific for the antigen.

In some embodiments, the disclosure provides a method of identifying a human immunoglobulin variable domain or CDR sequence (e.g., CDR3 sequence) of an antibody specific for an antigen, comprising: (i) obtaining a library of human immunoglobulin heavy chain and/or light chain variable domain sequences comprising a plurality of human immunoglobulin heavy chain and/or light chain variable domain sequences encoded by B cells of a rodent immunized with the antigen, and (ii) interrogating the library with a plurality of peptide sequences of human immunoglobulin heavy chain and/or light chain variable domains that were obtained from a sample comprising a population of antibodies produced by the rodent immunized with the antigen.

In some embodiments, the immunized rodent comprises in its germline genome: an immunoglobulin heavy chain variable region comprising one or more human heavy chain V gene segments, one or more human D gene segments, and one or more human heavy chain J gene segments, wherein the heavy chain variable region is operably linked to a constant region, and an immunoglobulin light chain variable region comprising one or more human light chain V gene segments and one or more human light chain J gene segments, wherein the light chain is operably linked to a constant region.

In some embodiments, the immunized rodent comprises in its germline genome a limited immunoglobulin light chain repertoire. In some embodiments, the immunized rodent comprises in its germline genome a single rearranged human light chain V/J. In some embodiments, the immunized rodent comprises in its germline genome two human light chain V gene segments and one or more human light chain J segments.

In some embodiments, the immunized rodent produces antibodies comprising two immunoglobulin heavy chains and two immunoglobulin light chains. In some embodiments, the immunized rodent does not produce single domain antibodies, heavy chain only antibodies, and/or nanobodies. In some embodiments, the immunized rodent comprises in its germline genome a limited immunoglobulin heavy chain repertoire, for example, a universal heavy chain.

In some embodiments, immunized rodent comprises in its germline genome a CH1 delete modification. In some embodiments, the immunized rodent produces single domain antibodies, a heavy chain only antibodies, and/or nanobodies.

In some embodiments, a first sample (i.e., a sample for sequence analysis) comprises a population of B cells from primary or secondary lymphoid organs, e.g., B cells from a bone marrow sample and/or a spleen sample, B cells from lymph nodes, B cells from Peyer's patches, etc. In some embodiments, the obtaining, from a first sample, a plurality of nucleic acid sequences that encode a plurality of immunoglobulin variable domains comprises preparing cDNA from the nucleic acid sequences and sequencing rearranged heavy chain VDJ sequences and/or rearranged light chain VJ sequences in the first sample. In some embodiments, obtaining a plurality of nucleic acid sequences that encode a plurality of immunoglobulin variable domains from the first sample comprises using DNA sequencing technology such as next generation DNA sequencing.

In some embodiments, a second sample (i.e., a sample for analysis of peptide sequences) is or comprises any bodily fluid comprising antibodies. In some embodiments, a second sample is or comprises serum, plasma, lymphoid organs, gut, cerebrospinal fluid, brain, spinal cord, placenta, or a combination thereof. In some embodiments, a second sample peptide sequences are obtained via mass spectrometric (MS) analysis (e.g., by combining liquid chromatography and mass spectrometry (LC-MS)) of the heavy and/or light chain variable domains of the population of antibodies in the second sample. Additionally, in some embodiments, prior to mass spectrometric analysis, a proteolytic digest of the heavy and/or light chain variable domains of the population of antibodies can be performed.

In some embodiments, a sample of antibodies for analysis of peptide sequences, may have been enriched ex vivo for one or more desired characteristics (e.g., prior to MS analysis). In some embodiments, obtaining a second sample further comprises depleting the second sample of antibodies not directed against the particular antigen. In some embodiments, obtaining a second sample further comprises enriching the second sample for antibodies directed against the particular antigen.

In some embodiments, interrogating the amino acid sequences of a plurality of immunoglobulin variable domains from a first sample with peptide sequences of heavy and/or light chain variable domains of a population of antibodies from a second sample comprises aligning the peptide sequences of heavy and/or light chain variable domains of the population of antibodies to the amino acid sequences of the plurality of immunoglobulin variable domains and, optionally, to each other.

In some embodiments, a method described herein comprises expressing an obtained nucleotide sequence encoding a human immunoglobulin variable domain in a second, recombinant antibody. In some embodiments, a nucleotide sequence encoding a human variable domain can be expressed in a cell line in operable linkage with a human immunoglobulin constant region. More specifically, in some embodiments, a human variable domain is a human heavy chain variable domain expressed in operable linkage with a human immunoglobulin heavy chain constant region to generate a human immunoglobulin heavy chain. In some embodiments, a human immunoglobulin heavy chain is expressed in a cell line with a human immunoglobulin light chain. In an embodiment where the human variable domain is a human light chain variable domain, it can be expressed in operable linkage with a human immunoglobulin light chain constant region to generate a human immunoglobulin light chain. In some embodiments, a human immunoglobulin light chain is expressed in a cell line with a human immunoglobulin heavy chain.

In some embodiments, a method described herein further comprises expressing an obtained nucleotide sequence encoding a human immunoglobulin variable domain in a recombinant antigen-binding protein.

In some embodiments, a recombinant antigen-binding protein is a human antibody, e.g., a human bispecific antibody.

In some embodiments, a recombinant antigen-binding protein is purified. In some embodiments, the affinity and/or specificity of a purified recombinant antigen-binding protein for the particular antigen is determined.

In some embodiments, a host is a genetically modified mouse that comprises in its genome (e.g., its germline genome) an immunoglobulin heavy chain variable region comprising one or more human heavy chain V gene segments, one or more human D gene segments, and one or more human heavy chain J gene segments, wherein the heavy chain variable region is operably linked to a murine constant region, and an immunoglobulin light chain variable region comprising one or more human light chain V gene segments and one or more human light chain J gene segments, wherein the light chain is operably linked to a murine constant region. In some embodiments, the immunoglobulin heavy chain variable region is operably linked to a mouse heavy chain constant region, and/or the immunoglobulin light chain variable region is operably linked to a mouse light chain constant region. Still further, an immunoglobulin heavy chain variable region may be operably linked to a mouse heavy chain constant region at the endogenous mouse heavy chain locus, and/or an immunoglobulin light chain variable region operably linked to a mouse light chain constant region is at the endogenous mouse light chain locus.

In some embodiments, a host is a genetically modified mouse that comprises in its genome, including in its germline genome, an immunoglobulin heavy chain variable region comprising a plurality of human heavy chain V gene segments, a plurality of human D gene segments, and a plurality of human heavy chain J gene segments, wherein the heavy chain variable region is operably linked to a murine heavy chain constant region, and an immunoglobulin light chain variable region comprising exactly two unrearranged human Vκ gene segments and five unrearranged human Jκ gene segments operably linked to a murine light chain constant region. In some embodiments, the exactly two unrearranged human Vκ gene segments are a human Vκ 1-39 gene segment and a human Vκ 3-20 gene segment.

In some embodiments, a host may be a genetically modified mouse whose genome (e.g., germline genome) comprises at an endogenous heavy chain locus: (i) an immunoglobulin heavy chain variable region comprising a plurality of unrearranged human V_{H} gene segments, a plurality of unrearranged human D_{H} gene segments, and a plurality of unrearranged human J_{H} gene segments operably linked to a mouse heavy chain constant region; (ii) a restricted unrearranged heavy chain variable region, comprising a single human V_{H} gene segment, one or more unrearranged human D_{H} gene segments, and one or more unrearranged human J_{H} gene segments, operably linked to a mouse heavy chain constant region; (iii) a universal heavy chain encoding sequence comprising a single rearranged human heavy chain variable region operably linked to a mouse heavy chain constant region; (iv) a histidine modified unrearranged heavy chain variable region, comprising one or more unrearranged human V_{H} gene segments, one or more unrearranged human D_{H} gene segments, and one or more unrearranged human J_{H} gene segments, further comprising substitution or insertion of at least one histidine for a non-histidine residue, operably linked to a mouse heavy chain constant region; (v) a heavy chain only immunoglobulin encoding sequence comprising an immunoglobulin heavy chain variable region, comprising one or more unrearranged human V_{H} gene segments, one or more unrearranged human D_{H} gene segments, and one or more unrearranged human J_{H} gene segments, operably linked to a heavy chain constant region wherein a non-IgM gene, e.g., an IgG gene, lacks a sequence that encodes a functional CH1 domain; or (vi) an engineered endogenous rodent immunoglobulin heavy chain locus comprising one or more unrearranged human V_{L} gene segments and one or more unrearranged human J_{L} gene segments, operably linked to a mouse immunoglobulin heavy chain constant region gene. In some embodiments, a host may be a genetically modified mouse whose genome (e.g., germline genome) comprises at an endogenous light chain locus: (i) an immunoglobulin light chain variable region comprising a plurality of unrearranged human Vκ gene segments and a plurality of unrearranged human Jκ gene segments operably linked to a mouse light chain constant region; (ii) a universal light chain encoding sequence comprising a single rearranged human light chain variable region, operably linked to mouse light chain constant region; (iii) a restricted light chain variable region, comprising two unrearranged human Vκ gene segments and one or more unrearranged human Jκ gene segments, operably linked to mouse light chain constant region; or (iv) a histidine modified light chain variable region comprising one or more human light chain V gene segments and one or more human light chain J gene segments, further comprising substitution or insertion of at least one histidine for a non-histidine residue, operably linked to mouse light chain constant region.

In some embodiments, a host comprises a functional ADAM6 gene, optionally wherein a host is a genetically modified mouse and a functional ADAM6 gene is a mouse ADAM6 gene. In some embodiments, a host may comprise and/or express an exogenous terminal deoxynucleotidyl transferase (TdT) gene.

The present disclosure also provides methods of obtaining an immunoglobulin variable domain or a CDR of an antibody specific for an antigen, comprising: interrogating peptide sequences of heavy and/or light chain variable domains of a population of antibodies from a sample obtained from a host immunized with the antigen, against a library of amino acid sequences comprising a plurality of human immunoglobulin variable domains, thereby obtaining a human immunoglobulin variable domain or CDR sequence of an antibody specific for the antigen. In some embodiments, the method comprises obtaining a sample comprising a population of antibodies directed against an antigen from a host immunized with the antigen. In some embodiments, the method comprises determining peptide sequences of heavy and/or light chain variable domains of the population of antibodies.

The present disclosure also provides methods for identifying a human immunoglobulin variable domain or CDR of an antibody specific for a particular antigen, the method comprising: comparing a plurality of amino acid sequences encoded by a plurality of nucleic acids that encode a plurality of human immunoglobulin variable domains produced by an animal immunized with said antigen with amino acid sequences comprising peptide fragments from light chain and/or heavy chain variable domains produced from a population of antibodies directed against the antigen; and thereby identifying a human immunoglobulin variable domain or CDR sequence of an antibody specific for said antigen.

In some embodiments, the immunized host is a genetically modified non-human mammal that comprises in its germline genome: an immunoglobulin heavy chain variable region comprising one or more human heavy chain V gene segments, one or more human D gene segments, and one or more human heavy chain J gene segments; wherein the heavy chain variable region is operably linked to a constant region, and an immunoglobulin light chain variable region comprising one or more human light chain V gene segments and one or more human light chain J gene segments; wherein the immunoglobulin light chain variable region is operably linked to a constant region.

In some embodiments, the present disclosure also provides methods of obtaining, from a host immunized with a particular antigen, a human immunoglobulin heavy chain variable domain or a CDR of an antibody specific for said antigen, comprising: obtaining amino acid sequences of a plurality of human immunoglobulin variable domains encoded by a plurality of nucleic acid sequences obtained from the host; determining peptide sequences of human heavy chain variable domains of a population of antibodies obtained from the immunized host; interrogating the amino acids sequences of the encoded plurality of human immunoglobulin heavy chain variable domains with the peptide sequences of the human heavy chain variable domains of the population of antibodies, thereby obtaining a human immunoglobulin heavy chain variable domain or a CDR of an antibody specific for the antigen. In some embodiments, the host is a genetically modified mouse that comprises in its genome, including in its germline genome: an immunoglobulin heavy chain variable region comprising a plurality of human heavy chain V gene segments, a plurality of human heavy chain D gene segments, and a plurality of human heavy chain J gene segments, wherein the heavy chain variable region is operably linked to a murine constant region, and an immunoglobulin light chain variable region which is a single rearranged human light chain variable region comprising a single human light chain V gene segment and a single human light chain J gene segment, wherein the human immunoglobulin light chain variable region is operably linked to a murine light chain constant region.

In some embodiments, a single rearranged human light chain variable region is a single rearranged human kappa light chain variable region comprising a single human light chain Vκ gene segment and a single human light chain Jκ gene segment. In some embodiments, a single human light chain Vκ gene segment is a Vκ1-39 or Vκ3-20 gene segment, and a single human light chain Jκ gene segment is a Jκ1 or a Jκ5 gene segment. In some embodiments, a single rearranged human kappa light chain variable region comprises a Vκ1-39 gene segment and a Jκ5 gene segment. In some embodiments, a single rearranged human kappa light chain variable region comprises a Vκ3-20 gene segment and a Jκ1 gene segment.

In some embodiments, a murine light chain constant region is a mouse kappa light chain constant region. In some embodiments, a single rearranged human light chain variable region is operably liked to a mouse kappa light chain constant region. In some embodiments, a single rearranged human light chain variable region is operably liked to a mouse kappa light chain constant region is at the endogenous mouse kappa light chain locus.

In some embodiments, a host comprises a functional ADAM6 gene or fragment thereof, optionally wherein a host is a genetically modified mouse and a functional ADAM6 gene is a mouse ADAM6 gene.

In some embodiments, a first sample comprises a population of B cells from primary or secondary lymphoid organs, e.g., B cells from a bone marrow sample and/or a spleen sample, B cells from lymph nodes, B cells from Peyer's patches, etc.

In some embodiments, obtaining from a first sample a plurality of nucleic acid sequences encoding a plurality of human immunoglobulin heavy chain variable domains comprises preparing cDNA from the nucleic acid sequences and sequencing rearranged heavy chain VDJ sequences in the first sample.

In certain embodiments, the plurality of nucleic acid sequences that encode a plurality of immunoglobulin variable domains obtained from the first sample is determined using DNA sequencing technology.

In some embodiments, a second sample is or comprises any bodily fluid comprising antibodies. In some embodiments, a second sample is or comprises serum, plasma, lymphoid organs, gut, cerebrospinal fluid, brain, spinal cord, or placenta. In some embodiments, determining peptide sequences from a second sample comprises mass spectrometric, e.g., including liquid chromatography and mass spectrometry (LC-MS), analysis of the heavy chain variable domains of the population of antibodies in the second sample. A method described herein may comprise, prior to mass spectrometric analysis, a proteolytic digest of the heavy chain variable domains of the population of antibodies.

In some embodiments, a method described herein comprises depleting the second sample of antibodies not directed against a particular antigen. In some embodiments, a method described herein comprises depleting the second sample of antibodies directed to a different antigen and/or a different epitope of the same antigen (e.g., that was used to immunize a host). In some embodiments, a method described herein comprises enriching a second sample for antibodies directed against the antigen of interest (e.g., that was used to immunize a host).

In some embodiments, interrogating amino acid sequences of a plurality of human immunoglobulin heavy chain variable domains with peptide sequences of human heavy chain variable domains of a population of antibodies comprises aligning the peptide sequences of heavy and/or light chain variable domains of the population of antibodies to the amino acid sequences of the plurality of immunoglobulin variable domains and, optionally, to each other.

In some embodiments, the present disclosure provides methods of identifying a human immunoglobulin variable domain or CDR sequence (e.g., CDR3 sequence) of an antibody specific for an antigen, comprising: (i) obtaining a plurality of peptide sequences of human immunoglobulin heavy chain and/or light chain variable domains that were obtained from a sample comprising a population of antibodies produced by a rodent immunized with the antigen, and (ii) interrogating a library of human immunoglobulin heavy chain and/or light chain variable domain sequences with the plurality of peptide sequences, wherein the library comprises a plurality of human immunoglobulin heavy chain and/or light chain variable domain sequences encoded by B cells of the immunized rodent, thereby obtaining a human immunoglobulin variable domain or CDR sequence of an antibody specific for the antigen.

In some embodiments, the present disclosure provides methods of identifying a human immunoglobulin variable domain or CDR sequence (e.g., CDR3 sequence) of an antibody specific for an antigen, comprising: (i) obtaining a library of human immunoglobulin heavy chain and/or light chain variable domain sequences comprising a plurality of human immunoglobulin heavy chain and/or light chain variable domain sequences encoded by B cells of a rodent immunized with the antigen, and (ii) interrogating the library with a plurality of peptide sequences of human immunoglobulin heavy chain and/or light chain variable domains that were obtained from a sample comprising a population of antibodies produced by the rodent immunized with the antigen.

In some embodiments, an immunized rodent comprises in its germline genome an immunoglobulin heavy chain variable region comprising a plurality of human heavy chain V gene segments, a plurality of human D gene segments, and a plurality of human heavy chain J gene segments, and an immunoglobulin light chain variable region comprising: (i) a universal light chain encoding sequence comprising a rearranged human light chain variable region comprising a single human V_{L} gene segment and single human light J_{L} gene segment, operably linked to a mouse light chain constant region; (ii) a restricted light chain variable region, comprising two unrearranged human V_{L} gene segments and one or more unrearranged human J_{L} gene segments, operably linked to a mouse light chain constant region; or (iii) a histidine modified light chain variable region comprising one or more human light chain V gene segments and one or more human light chain J gene segments, further comprising substitution or insertion of at least one histidine for a non-histidine residue, operably linked to a mouse light chain constant region. In some embodiments, provided methods comprise obtaining a library of human immunoglobulin heavy chain variable domain sequences comprising a plurality of human immunoglobulin heavy chain variable domain sequences encoded by B cells of a rodent immunized with the antigen, and (ii) interrogating the library with a plurality of peptide sequences of human immunoglobulin heavy chain variable domains that were obtained from a sample comprising a population of antibodies produced by the rodent immunized with the antigen.

In some embodiments, an immunized rodent comprises in its germline genome an immunoglobulin light chain variable region comprising a plurality of unrearranged human V_{L} gene segments and a plurality of unrearranged human J_{L} gene segments operably linked to a mouse light chain constant region and an immunoglobulin heavy chain variable region comprising: (i) a restricted unrearranged heavy chain variable region, comprising a single human V_{H} gene segment, one or more unrearranged human D_{H} gene segments, and one or more unrearranged human J_{H} gene segments, operably linked to a mouse heavy chain constant region; (ii) a universal heavy chain encoding sequence comprising a single rearranged human heavy chain variable region comprising a single human V_{H} gene segment, a single human D_{H} gene segment, and a single human J_{H} gene segment, operably linked to a mouse heavy chain constant region; or (iii) a histidine modified unrearranged heavy chain variable region, comprising one or more unrearranged human V_{H} gene segments, one or more unrearranged human D_{H} gene segments, and one or more unrearranged human J_{H} gene segments, further comprising substitution or insertion of at least one histidine for a non-histidine residue, operably linked to a mouse heavy chain constant region. In some embodiments, provided methods comprise obtaining a library of human immunoglobulin light chain variable domain sequences comprising a plurality of human immunoglobulin light chain variable domain sequences encoded by B cells of a rodent immunized with the antigen, and (ii) interrogating the library with a plurality of peptide sequences of human immunoglobulin light chain variable domains that were obtained from a sample comprising a population of antibodies produced by the rodent immunized with the antigen.

In some embodiments, a method described herein can comprise obtaining a nucleotide sequence of a human heavy chain variable domain of an antibody specific for the antigen and expressing the obtained nucleotide sequence encoding the human immunoglobulin heavy chain variable domain in an antigen-binding protein. In some embodiments, an antigen-binding protein is a second (e.g., recombinant) antibody.

In some embodiments, a nucleotide sequence encoding a human heavy chain variable domain is expressed in a cell line in operable linkage with a human immunoglobulin heavy constant region to generate a human immunoglobulin heavy chain. In some embodiments, a human immunoglobulin heavy chain may be expressed in a cell line with a human immunoglobulin light chain. In some embodiments, a human immunoglobulin light chain may be derived from the same single rearranged variable region sequence as present in the mouse, or a somatically mutated version thereof.

In some embodiments, a method described herein comprises expressing an obtained nucleotide sequence encoding a human immunoglobulin variable domain in a recombinant antigen-binding protein. In some embodiments, a recombinant antigen-binding protein is a second, recombinant antibody. In some embodiments, a second antibody is a human antibody and may be a bispecific antibody. A second antibody may be purified and affinity and/or specificity of the purified second antibody determined for the particular antigen.

In some embodiments, a sample for determining peptide sequences of heavy and/or light chain variable domains is or comprises any bodily fluid comprising antibodies. In some embodiments, a second sample is or comprises serum, plasma, lymphoid organs, gut, cerebrospinal fluid, brain, spinal cord, or placenta, or a combination thereof. In some embodiments, determining peptide sequences of heavy and/or light chain variable domains comprises MS analysis (e.g., LC/MS analysis). In some embodiments, determining peptide sequences of heavy and/or light chain variable domains comprises MS analysis (e.g., LC/MS analysis) of a sample comprising antibodies obtained from a host immunized with an antigen.

In some embodiments, a library of amino acid sequences comprising a plurality of human immunoglobulin variable domains is encoded by a plurality of nucleic acids obtained from the host immunized with the antigen. In some embodiments, a library of amino acid sequences comprising a plurality of human immunoglobulin variable domains is encoded by a plurality of nucleic acids obtained from a B cells sample such as a bone marrow and/or a spleen sample.

These and other features and advantages provided in the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTIONS OF THE DRAWINGS

FIG. 1 includes a schematic overview of an exemplary method for obtaining antibodies using LC-MS in tandem with next generation sequencing for an exemplary antigen of interest.

FIGS. 2A and 2B include graphs showing diversity, depicted as % sequences (Y axis), in human heavy chain V (FIG. 2A) and J (FIG. 2B) gene usage in IgGs obtained from spleen and bone marrow of a mouse donor immunized with CD22.

FIGS. 3A and 3B show HCDR3 overlap in (FIG. 3A) spleens from different mice (˜2% overlap) and in (FIG. 3B) bone marrow and spleen from the same mouse (10-14% overlap) as determined by Next Generation Sequencing analysis.

FIG. 4 shows an example of the selection of anti-CD22 antibody based on the mass spectra match and the NGS count from a group of Abs containing homologous CDR3 sequence. At the top of FIG. 4 is a sequence of an anti-CD22 antibody heavy chain variable domain; dashed boxes delineate the CDR1, CDR2 and CDR3 sequences (respectively, from left to right). Underlining indicates the sequence coverage from mass spectrometry analysis, with 100% coverage of CDR1, 0% coverage of CDR2, and 100% coverage of CDR3.

FIG. 5 shows diversification of antibodies based on the depicted CDR3 sequences obtained from universal light chain mice. Antibodies were grouped based on differences in their CDR3 sequences, and diverse repertoire was selected for further cloning and characterization.

### DETAILED DESCRIPTION

The disclosure provides methods for obtaining antibodies with human variable domains using a combination of mass spectrometry and next generation sequencing. The disclosure further provides methods for making antibodies.

### Certain Definitions

As utilized in accordance with the present disclosure, the following terms, unless otherwise indicated, shall be understood to have the following meanings. Unless otherwise required by context, singular terms shall include pluralities and plural terms shall include the singular.

Additionally, singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a meaningful range of a value. The allowable variation encompassed by the term “about” or “approximately” depends on the particular system under study, and can be readily appreciated by one of ordinary skill in the art.

The term “antigen” refers to any agent (e.g., protein, peptide, polysaccharide, lipid, glycoprotein, glycolipid, nucleotide, nucleic acid, polymer, and/or portions or combinations thereof) that, when introduced into an immunocompetent host is recognized by the immune system of the host and elicits an immune response by the host. In some embodiments, an antigen elicits a humoral response (e.g., including production of antigen-specific antibodies).

The terms “antibody”, “antigen-binding protein” or “epitope binding protein” and the like, refer to monoclonal antibodies, IgA, IgG, IgE or IgM antibodies, multi-specific antibodies, human antibodies, humanized antibodies, chimeric antibodies, reverse chimeric antibodies, antibodies with light chain variable gene segments on heavy chain, antibodies with heavy chain variable gene segments on light chain, as well as, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. Thus, “antigen binding fragment” and “antigen-binding portion” and “epitope-binding fragment” of an antigen binding molecule are also encompassed herein, and refer to fragments that retain the ability to bind to an antigen. The term “antigen-binding protein” also includes, for example, single domain antibodies, heavy chain only antibodies, covalent diabodies such as those disclosed in U.S. Pat. Appl. Pub. 20070004909, incorporated herein by reference in its entirety, and Ig-DARTS such as those disclosed in U.S. Pat. Appl. Pub. 20090060910, incorporated herein by reference in its entirety. In some certain embodiments, an antibody is a canonical antibody that includes at least two heavy (H) chains and two light (L) chains (e.g., inter-connected by disulfide bonds).

The term “specifically binds,” “binds in a specific manner,” “antigen-specific” or the like, indicates that the molecules involved in the specific binding are (1) able to stably bind to each other (e.g., associate, e.g., form intermolecular non-covalent bonds), under physiological conditions, and are (2) unable to stably bind under physiological conditions to other molecules outside the specified binding pair. Specific binding may also be characterized by an equilibrium dissociation constant (K_{D}) from the low micromolar to the picomolar range. High specificity may be in the low nanomolar range, with very high specificity being in the picomolar range. Methods for determining whether two molecules specifically bind are well known in the art and include, for example, equilibrium dialysis, and surface plasmon resonance.

“Host” refers to an animal or non-human mammal that produces immune system proteins in response to foreign molecules or antigens introduced into the host via injection or other suitable route. Introduction of an antigen or other foreign matter into the host elicits antibody production and associated immune responses.

The term “non-human mammal” and the like refer to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human animal is a mammal. In some embodiments, a non-human mammal is a primate, a goat, a sheep, a pig, a dog, a cow, or a rodent. Various non-human animals are additionally described herein below. Further, the term “genetically modified non-human mammal” as used herein refers to a “non-human mammal” as described above wherein the genetic material of the non-human mammal has been altered using genetic engineering techniques, for example, to introduce, delete, enhance, suppress, or mutate the genetic sequence of the non-human mammal.

The terms “humanized,” “chimeric,” “human/non-human,” and the like, are commonly used to refer to antibodies (or antigen-binding proteins, or antibody components) that include a sequence (e.g., a nucleic acid, protein, etc.) wherein at least a portion of the sequence is derived from a human or where at least a portion of the sequence was non-human in origin (e.g., of a rodent, e.g., of a mouse), has been replaced with a corresponding portion of a corresponding human antibody (or antigen-binding proteins, or antibody components) sequence in such a manner that the modified (e.g., humanized, chimeric, human/non-human, etc.) molecule retains its biological function and/or maintains the structure that performs the retained biological function. For example, a chimeric antibody includes V_{H} and V_{L} region sequences that are found in a first species (e.g., a human) and constant region sequences that are found in a second, different species (e.g., a non-human animal, e.g., a rodent, e.g., a mouse). In some embodiments, an antibody with human V_{H} and V_{L} regions linked to non-human constant regions (e.g., a mouse constant region) is referred to as a “reverse chimeric antibody”. In contrast, “human” antibodies and the like encompass sequences having only a human origin (e.g., human nucleotide and/or protein sequences).

The terms “genetically modified non-human animal” and “genetically engineered non-human animal” are used interchangeably herein and refer to any non-naturally occurring non-human animal (e.g., a rodent, e.g., a rat or a mouse) in which one or more of the cells of the non-human animal contain heterologous nucleic acid and/or a gene or genes encoding a polypeptide of interest, in whole or in part. For example, in some embodiments, a “genetically modified non-human animal” or “genetically engineered non-human animal” refers to non-human animal that contains a transgene or transgene construct as described herein. In some embodiments, a heterologous nucleic acid and/or gene is introduced into the cell, directly or indirectly by introduction into a precursor cell, by way of deliberate genetic manipulation, such as by microinjection or by infection with a recombinant virus. The term genetic manipulation does not include classic breeding techniques, but rather is directed to introduction of recombinant DNA molecule(s). This molecule may be integrated within a chromosome. The phrases “genetically modified non-human animal” or “genetically engineered non-human animal” refers to animals that are heterozygous or homozygous for a heterologous nucleic acid and/or gene, and/or animals that have single or multiple copies of a heterologous nucleic acid and/or gene.

The term “germline configuration” as used herein, refers to an arrangement of sequences (e.g., gene segments) as found in an endogenous germline genome of a wild-type animal (e.g., mouse, rat, or human). Examples of germline configurations of immunoglobulin gene segments can be found, e.g., in LeFranc, M-P., The Immunoglobulin FactsBook, Academic Press, May 23, 2001 (referred to herein as “LeFranc 2001”):
An exemplary configuration of human heavy chain variable region gene segments and human heavy chain constant region genes can be found at p. 47 of LeFranc 2001;
An exemplary configuration of human λ light chain variable region gene segments and human λ light chain constant region genes can be found at p. 61 of LeFranc 2001;
An exemplary configuration of human κ light chain variable region gene segments and human κ light chain constant region genes can be found at p. 53 of LeFranc 2001;
An exemplary configuration of mouse heavy chain variable region gene segments and mouse heavy chain constant region genes can be found at Lucas, J. et al., Chapter 1: The Structure and Regulation of the Immunoglobulin Loci, Molecular Biology of B Cells, 2nd Edition, Academic Press, 2015 (Lucas);
An exemplary configuration of mouse λ light chain variable region gene segments and mouse λ light chain constant region genes can be found at LeFranc, M-P et al., Chapter 4: Immunoglobulin Lambda (IGL) Genes of Human and Mouse, Molecular Biology of B Cells, 1^{st} Edition, Academic Press, 2004 (LeFranc 2004); and
An exemplary configuration of mouse κ light chain variable region gene segments and mouse κ light chain constant region genes can be found at Christele, M-J, et al., Nomenclature and Overview of the Mouse (Mus musculus and Mus sp.) Immunoglobulin Kappa (IGK) Genes, Exp Clin Immunogenet 2001, 18:255-279 (Christele).

Each of the cited sections of LeFranc 2001, Lucas, LeFranc 2004, and Christele listed above are incorporated herein by reference.

The term “germline genome” as used herein, refers to the genome found in a germ cell (e.g., a gamete, e.g., a sperm or egg) used in the formation of an animal. A germline genome is a source of genomic DNA for cells in an animal. As such, an animal (e.g., a mouse or rat) having a modification in its germline genome is considered to have the modification in the genomic DNA of all of its cells.

The term “germline sequence” as used herein, refers to a DNA sequence as found in an endogenous germline genome of a wild-type animal (e.g., mouse, rat, or human), or an RNA or amino acid sequence encoded by a DNA sequence as found in an endogenous germline genome of an animal (e.g., mouse, rat, or human). Representative germline sequences of immunoglobulin gene segments can be found, e.g., in LeFranc 2001:
Representative germline nucleotide sequences of human V_{H} gene segments and representative germline amino acid sequences of human V_{H} gene segments, which can be utilized in some embodiments as described herein, can be found pages 107-234 of LeFranc 2001;
Representative germline nucleotide sequences of human D gene segments and representative germline amino acid sequences of human D gene segments, which can be utilized in some embodiments as described herein, can be found pages 98-100 of LeFranc 2001;
Representative germline nucleotide sequences of human J_{H} gene segments and representative germline amino acid sequences of human J_{H} gene segments, which can be utilized in some embodiments as described herein, can be found page 104 of LeFranc 2001;
Representative germline nucleotide sequences of human Vλ gene segments and representative germline amino acid sequences of human Vλ gene segments, which can be utilized in some embodiments of a non-human animal as described herein, can be found pages 350-428 of LeFranc 2001; and
Representative germline nucleotide sequences of human Jλ gene segments and representative germline amino acid sequences of human Jλ gene segments, which can be utilized in some embodiments of a non-human animal as described herein, can be found pages 346 of LeFranc 2001.

Each of the cited sections of LeFranc 2001 listed above are incorporated herein by reference.

The phrase “complementarity determining region,” or the term “CDR,” includes an amino acid sequence encoded by a nucleic acid sequence of an organism's immunoglobulin genes that normally (i.e., in a wild-type animal) appears between two framework (FR) regions in a variable domain of a light or a heavy chain of an immunoglobulin molecule (e.g., an antibody). A CDR can be encoded by, for example, a germline sequence or a rearranged or unrearranged sequence, and, for example, by a naive or a mature B cell. A CDR can be somatically mutated (e.g., vary from a sequence encoded in an animal's germline), humanized, and/or modified with amino acid substitutions, additions, or deletions. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of connecting the sequences (e.g., V-D-J recombination to form a heavy chain CDR3). Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

The phrase “gene segment,” or “segment” includes reference to a variable (V) gene segment (e.g., an immunoglobulin light chain variable (V_{L}) gene segment or an immunoglobulin heavy chain variable (V_{H}) gene segment), an immunoglobulin heavy chain diversity (D) gene segment, or a joining (J) gene segment, e.g., an immunoglobulin light chain joining (J_{L}) gene segment or an immunoglobulin heavy chain joining (JO gene segment, which includes unrearranged sequences at immunoglobulin loci that can participate in rearrangement (mediated by, e.g., endogenous recombinases) to form a rearranged light chain V_{L}/J_{L} or rearranged heavy chain V_{H}/D/J_{H} sequence. Unless indicated otherwise, the unrearranged V, D, and J segments are associated with recombination signal sequences (RSS) that allow for V_{L}/J_{L} recombination or V_{H}/D_{H}/J_{H} recombination according to the 12/23 rule.

The term “rearranged” as used herein, describes a DNA sequence that includes two or more immunoglobulin gene segments joined (directly or indirectly) together, such that the joined gene segments together have a DNA sequence that encodes a variable region of an immunoglobulin. The two or more immunoglobulin gene segments of a rearranged DNA sequence are no longer associated with functioning recombination signal sequences (RSS), and as such cannot undergo further rearrangement. Those of skill in the art will recognize that, while two or more immunoglobulin gene segments of a rearranged DNA sequence may not be able to rearrange further, it does not mean that other immunoglobulin gene segments within the same locus cannot undergo, e.g., secondary rearrangement. Those of skill in the art will appreciate that rearranged gene segments (e.g., in a rearranged immunoglobulin variable region) can be joined together via a natural VDJ recombination process. Those of skill in the art will also appreciate that rearranged gene segments (e.g., in a rearranged immunoglobulin variable region) can be engineered to be joined together, e.g., by joining the gene segments using standard recombinant techniques. Rearranged immunoglobulin variable regions typically include two or more joined immunoglobulin gene segments. For example, a rearranged immunoglobulin λ light chain variable region can include a Vλ gene segment joined with a Jλ gene segment. A rearranged immunoglobulin heavy chain variable region can include a V_{H} gene segment, a D gene segment, a J_{H} gene segment that are joined. Those of skill in the art will also appreciate that all or substantially all intergenic sequence is generally removed between immunoglobulin gene segments in a rearranged immunoglobulin variable region. Those of skill in the art will further appreciate that a rearranged sequence can include, among other things, introns in the gene segments.

The term “unrearranged” as used herein, describes a DNA sequence that includes two or more immunoglobulin gene segments that have not undergone a recombination event or otherwise been joined, and therefore, include intergenic sequence(s) between them. Those of skill in the art will appreciate that unrearranged V gene segments and J gene segments can be associated with an intact recombination signal sequence (RSS). Unrearranged D gene segments can be flanked by two intact recombination signal sequences (RSSs). Those of skill in the art will further appreciate that unrearranged gene segments (e.g., unrearranged V gene segments) can include, among other things, introns.

The term “protein” or interchangeably, “polypeptide” is used herein encompasses all kinds of naturally occurring and synthetic proteins, including protein fragments of all lengths, peptides, fusion proteins and modified proteins, including without limitation, glycoproteins, as well as all other types of modified proteins (e.g., including but not limited to proteins resulting from phosphorylation, acetylation, myristoylation, palmitoylation, glycosylation, oxidation, formylation, amidation, polyglutamylation, ADP ribosylation, pegylation, and biotinylation).

The terms “nucleic acid” and “nucleotide” encompass both DNA and RNA unless specified otherwise. In particular, the terms “nucleic acid” and “nucleotide sequence” are used herein interchangeably.

The term “operably linked” or the like refers to a juxtaposition wherein the components described are in a relationship permitting them to function in their intended manner. For example, unrearranged variable region gene segments are “operably linked” to a contiguous constant region gene if the unrearranged variable region gene segments are capable of rearranging to form a rearranged variable region gene that is expressed in a B cell or its progenitor cells in conjunction with the constant region gene as a polypeptide chain of an antigen binding protein. A control sequence “operably linked” to a coding sequence is positioned in such a way that expression of the coding sequence is achieved under conditions compatible with the control sequences. “Operably linked” sequences include both expression control sequences that are contiguous with a gene of interest and expression control sequences that act in trans or at a distance to control a gene of interest (or sequence of interest). The term “expression control sequence” includes polynucleotide sequences, which are necessary to affect the expression and processing of coding sequences to which they are ligated. “Expression control sequences” include: appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance polypeptide stability; and when desired, sequences that enhance polypeptide secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequences. The term “control sequences” is intended to include components whose presence is essential or beneficial for expression and processing and can also include additional components whose presence is advantageous, for example, leader sequences.

The term “heterologous” refers to an agent or entity from a different source. For example, when used in reference to a polypeptide, gene, or gene product present in a particular cell or organism, the term clarifies that the relevant polypeptide, gene, or gene product: 1) was engineered by the hand of man; 2) was introduced into the cell or organism (or a precursor thereof) through the hand of man (e.g., via genetic engineering); and/or 3) is not naturally produced by or present in the relevant cell or organism (e.g., the relevant cell type or organism type). “Heterologous” also includes a polypeptide, gene or gene product that is normally present in a particular native cell or organism, but has been altered or modified, for example, by mutation or placement under the control of non-naturally associated and, in some embodiments, non-endogenous regulatory elements (e.g., a promoter).

An antibody “heavy chain” typically includes an immunoglobulin heavy chain variable domain and an immunoglobulin heavy chain constant domain. A variable domain can be further subdivided into regions of hypervariability, termed complementarity determining regions (CDR), interspersed with regions that are more conserved, termed framework regions (FR). Heavy chain variable domains include three heavy chain CDRs and four FR regions (e.g., FR1-CDR1-FR2-CDR2-FR3-CDR3-FR4), unless otherwise specified. Fragments of heavy chains include CDRs, CDRs and FRs, and combinations thereof. Generally, a full-length heavy chain comprises, from N-terminal to C-terminal, the following: a heavy chain variable domain that includes FR1-CDR1-FR2-CDR2-FR3-CDR3-FR4, a CH1 domain, a hinge, a CH2 domain, and a CH3 domain. In some embodiments, a full-length heavy chain also comprises a CH4 domain (e.g., IgE and IgM isotype antibodies). A functional fragment of a heavy chain includes a fragment that is capable of specifically recognizing an epitope (e.g., recognizing the epitope with a K_{D} in the micromolar, nanomolar, or picomolar range), that is capable of expressing and secreting from a cell, and that comprises at least one CDR.

The phrase “light chain” includes an immunoglobulin light chain sequence from any organism, and unless otherwise specified, includes human κ and λ light chains, as well as surrogate light chains (e.g., comprising VpreB, λ5, etc.) Light chain variable domains typically include three light chain CDRs and four framework (FR) regions, unless otherwise specified. Generally, a full-length light chain includes, from amino terminus to carboxyl terminus, a V_{L} domain that includes FR1-CDR1-FR2-CDR2-FR3-CDR3-FR4, and a light chain constant domain. Light chains include those, e.g., that do not selectively bind either a first or a second epitope selectively bound by the epitope-binding protein in which they appear. Light chains also include those that bind and recognize, or assist the heavy chain with binding and recognizing, one or more epitopes selectively bound by the epitope-binding protein in which they appear. Examples of light chains include universal or common light chains, e.g., those derived from a single rearranged human light chain variable region such as a human Vκ1-39Jκ5 or a human Vκ3-20Jκ1, as described herein, and include somatically mutated (e.g., affinity matured) versions of the same.

The phrase “derived from” when used concerning a rearranged variable region gene or a variable domain “derived from” an unrearranged variable region and/or unrearranged variable region gene segments refers to the ability to trace the sequence of the rearranged variable region gene or variable domain back to a set of unrearranged variable region gene segments that were rearranged to form the rearranged variable region gene that expresses the variable domain (accounting for, where applicable, splice differences and somatic mutations). For example, a rearranged variable region gene that has undergone somatic hypermutation does not change the fact that it is derived from the unrearranged variable region gene segments. In addition, the phrase “derived from” in the context of universal light chain can refer to ability to trace back the expressed antibody sequence to the universal or single rearranged light chain present in the genome of the mouse; such light chain derived from the single rearranged light chain sequence in the genome may differ from the single rearranged light chain sequence through somatic hypermutations.

As used herein, the term “locus” refers to a region on a chromosome that contains a set of related genetic elements (e.g., genes, gene segments, or regulatory elements). For example, an unrearranged immunoglobulin locus may include immunoglobulin variable region gene segments, one or more immunoglobulin constant region genes and associated regulatory elements (e.g., promoters, enhancers, switch elements, etc.) that direct V(D)J recombination and immunoglobulin expression. A locus can be endogenous or non-endogenous. The term “endogenous locus” refers to a location on a chromosome at which a particular genetic element is naturally found.

In accordance with the disclosure herein, there can be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994, each of which publications is incorporated herein in its entirety by reference. These techniques include site directed mutagenesis, see, e.g., in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932, 419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26:1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218; each of which publications is incorporated herein in its entirety by reference.

### Methods for Identification of Antigen-Specific Antibodies

The present disclosure provides methods for identifying and/or selecting a sequence of an antigen-binding protein (e.g., an antibody) with a human variable domain. Various methods described herein utilize nucleic acid sequencing and mass spectroscopy (MS) to select antibody sequences (e.g., variable domain sequences or CDR sequences) that bind a particular antigen. In exemplary embodiments, LC-MS and next generation sequencing (NGS) are used to select antibody or variable domain sequences from a plurality of variable domain sequences. In some embodiments, the LC-MS and NGS utilize information about a human immunoglobulin variable domain to identify and obtain antibodies directed against a given antigen. In some embodiments, a complementarity determining region 3 (CDR3) of the antibodies of interest is identified and obtained.

In various embodiments, methods described herein allow identification of antigen-specific antibody sequences from genetically modified non-human animals that may not be easily detected, e.g., via conventional methods. Known methods for antibody identification from genetically modified animals commonly rely on the presence of viable B cells, and/or expression of antibodies on the surface of a B cell (e.g., via hybridoma technology). Methods provided herein allow for identification/isolation of antibodies in the absence of viable cells (e.g., B cells). In some embodiments, methods provided herein allow for identification/isolation of secreted antibodies, e.g., in serum. Methods provided herein also allow identification of antibodies from antibody sources that are not typically used in conventional antibody identification methods.

In some embodiments, methods provided herein can be used in conjunction with conventional antibody identification/isolation methods in order to enrich and/or increase the pool of antibodies obtained against the antigen of interest from a genetically modified animal. For example, methods described herein may be used in conjunction with hybridoma technology, or in conjunction with a method that involves direct isolation from antigen-positive B cells, see, e.g., U.S. Pat. No. 7,582,298, incorporated herein by reference in its entirety.

The adaptive immune response is highly specific and serves as a long-term immune defense that retains memory for future antigen encounters. The adaptive immune response is antigen specific and mediated, in-part, by V(D)J recombination or rearrangement. Immunoglobulin V(D)J recombination occurs in developing B cells of the bone marrow and allows for recognition of a wide array of antigens. VDJ rearrangement is the rearrangement of variable (V), joining (J), and diversity (D) gene segments in the heavy chain of immunoglobulins. The process is similar for the light chain, however the light chain lacks D gene segments, and thus only undergoes VJ rearrangement.

Importantly V(D)J recombination, and other processes of antibody diversification such as junctional nucleotide addition/subtraction and somatic hypermutation, generate a large repertoire of antibodies from a limited number of genes. These processes allow for generation of specific high affinity antibodies against a variety of antigens. This ability to generate antibodies has been harnessed in genetically modified animals to generate therapeutic antibodies against human targets. Genetically modified mice comprising human V(D)J gene segments (e.g., those described in U.S. Pat. Nos. 5,633,425, 5,770,429, 5,814,318, 6,075,181, 6,114,598, 6,150,584, 6,998,514, 7,795,494, 7,910,798, 8,232,449, 8,703,485, 8,907,157, and 9,145,588, each of which is hereby incorporated by reference in its entirety, as well as in U.S. Pat. Pub. Nos. 2008/0098490, 2010/0146647, 2013/0145484, 2012/0167237, 2013/0167256, 2013/0219535, 2012/0207278, and 2015/0113668, each of which is hereby incorporated by reference in its entirety, and in PCT Pub. Nos. WO2007117410, WO2008151081, WO2009157771, WO2010039900, WO2011004192, WO2011123708, WO2014093908, WO2014093908, WO2006008548, WO2010109165, WO2016062990, WO2018039180, WO2011158009, WO2013041844, WO2013041846, WO2013079953, WO2013061098, WO2013144567, WO2013144566, WO2013171505, WO2019008123, and WO2020169022, each of which are hereby incorporated by reference in its entirety) are immunized against the antigen of interest, and antigen-specific antibodies are identified, purified and then screened for desired therapeutic properties. Other genetically modified mice comprising human V(D)J gene segments (e.g., those described in U.S. Pat. Nos. 6,596,541, 6,586,251, 8,642,835, 9,706,759, 10,238,093, 8,754,287, 10,143,186, 9,796,788, 10,130,081, 9,226,484, 9,012,717, 10,246,509, 9,204,624, and 9,686,970, and each of which is hereby incorporated by reference in its entirety, as well as in U.S. Pat. Pub. Nos. 2013/0212719, 2015/0289489, 2017/0347633, 2019/0223418, 2018/0125043, 2019/0261612, and 2019/0380316, each of which is hereby incorporated by reference in its entirety, in PCT Pub. Nos. WO2013138680, WO2013138712, WO2013138681, WO2015042250, WO2012148873, WO2013134263, WO2013184761, WO2014160179, WO2017214089, WO2016149678, and WO2017123808, and Murphy, A., “VelocImmune: Immunoglobulin Variable Region Humanized Mouse,” in Recombinant Antibodies for Immunotherapy, New York, N.Y., Cambridge University Press, 101-107 (2009), each of which are hereby incorporated by reference in its entirety) are immunized against the antigen of interest, and antigen-specific antibodies are identified, purified and then screened for desired therapeutic properties. Detailed embodiments of certain exemplary genetically engineered non-human animals, e.g., rodents, e.g., rats or mice, that may be used in the methods described herein, are further detailed in a separate section below. Various embodiments of the present invention allow obtaining therapeutic antibodies with desired properties from secreted antibody molecules obtained directly from the immunized animal. Obtaining secreted antibody molecules does not require presence of viable cells that express antibodies on the cell surface. As described herein, obtaining the antibodies with desired properties, from the population of antibodies, can be achieved using mass spectrometry, as discussed herein.

In various embodiments described herein, the antibody obtained/identified by the methods can be an antibody of any isotype, e.g., IgM, IgD, IgG, IgA, and IgE. In some embodiments, the antibody obtained/identified by the methods is of IgG isotype. In other embodiments, the antibody obtained/identified by the methods is of IgM isotype.

In some embodiments, an antibody or antigen-binding protein obtained/identified by the methods provided herein is not a single domain antibody, a heavy chain only antibody and/or a nanobody.

In various embodiments, provided herein are methods of obtaining a human immunoglobulin variable domain of an antibody specific for said antigen, comprising: obtaining a plurality of nucleic acid sequences that encode a plurality of immunoglobulin variable domains obtained from a first sample from a host immunized with a particular antigen; determining peptide sequences of heavy and/or light chain variable domains of a population of antibodies obtained from a second sample from the host comprising a population of antibodies directed against the antigen; interrogating the amino acid sequences of the encoded plurality of immunoglobulin variable domains with the peptide sequences of heavy and/or light chain variable domains of the population of antibodies, thereby obtaining a human immunoglobulin variable domain of an antibody specific for the antigen. In some embodiments, interrogation comprises aligning peptide sequences of heavy and/or light chain variable domains of the population of antibodies to each other and to amino acid sequences of the plurality of immunoglobulin variable domains.

In various embodiments, the method further comprises obtaining a nucleotide sequence of the human variable domain of the antibody specific for the antigen. Due to the degeneracy of the genetic code, multiple nucleotide sequences may encode the human variable domain of the antibody specific for the antigen, and in some embodiments describe herein, a nucleotide sequence may be optimized, e.g., for expression in a cell, e.g., for expression in a mammalian cell.

Samples for Sequencing

The present disclosure encompasses a recognition that information about particular antibodies that have certain binding properties can be identified using NGS and MS techniques, as described further herein. While the source of nucleic acids encoding antibodies and antibodies themselves for use in methods described herein is not restricted to animals, methods disclosed herein are particularly advantageous when an animal (e.g., a genetically modified animal as described herein) is the source of both the nucleic acid sample and the antibody sample. Nonetheless, methods described herein can also be used with other antibody platform technologies or other antibody expression technologies, including those using, e.g., phage display or intelligent design approaches.

Moreover, the present disclosure provides the recognition that antibodies derived from a restricted heavy or light chain variable sequence allow simplification of NGS and MS analyses, as the analyses can be focused on variable domain or CDR, e.g., CDR3, repertoire determination of solely the nonrestricted immunoglobulin chain. The present disclosure also recognizes that antibodies derived from a restricted heavy or light chain variable sequence can be obtained from genetically modified non-human animals, e.g., those non-human animals comprising a restricted heavy or light chain variable sequence. Such animals provide, e.g., a benefit in that the antibodies they produce have gone through natural immune system processes, and therefore, among other things, can have an increased chance of exhibiting high-affinity and specific binding while also having a decreased chance of being immunogenic.

In some embodiments, antibody sequences analyzed by NGS comprise a population of antibodies with a restricted light chain repertoire, e.g., a population of universal light chain antibodies. In some embodiments, antibody sequences analyzed by NGS comprise a population of antibodies with a restricted heavy chain repertoire, e.g., a population of universal heavy chain antibodies.

Even so, current technology allows identification of full length heavy and light chains in a plurality of immunoglobulin molecules using single cell sequencing approaches (see, e.g., DeKosky et al. (2015) Nat. Med. 21(1):85-91; Goldstein et al. (2019) Commun. Biol. 2:304; and Singh et al. (2019) Nat. Commun. 10(1):3120; incorporated herein by reference in their entirety); therefore, in some embodiments, a plurality of nucleic acid sequences that encode a plurality of immunoglobulin heavy and light chain variable domains may be obtained simultaneously from the first sample using single B cell next generation sequencing approaches, and thus, the method may encompass identification from a non-human animal host without restriction of a light or heavy chain sequence.

In some embodiments, the antigen of interest is a disease-associated antigen. In some embodiments, the disease-associated antigen is a tumor antigen. Various tumor antigens are listed in the database of T cell defined tumor antigens (van der Bruggen P, Stroobant V, Vigneron N, Van den Eynde B. Peptide database: T cell-defined tumor antigens. Cancer Immun 2013). In some other embodiments, the antigen of interest is an infectious disease antigen, e.g., a viral antigen or a bacterial antigen. A non-human animal may be immunized with an antigen of interest in a DNA or protein form, using techniques known in the art.

In some embodiments, a first sample comprises a population of B cells. In some embodiments, the population of B cells is isolated from a bone marrow sample and/or a spleen sample. In additional embodiments, the first sample may be obtained from other lymphoid organs, e.g., lymph nodes, Peyer's patches in the gut, etc.

One of skill in the art will understand that “B cell” may refer to a wide range of B-cell subtypes including, but not limited to, plasmablasts, plasma cells (e.g., long-lived plasma cells), memory B-cells, and B-2 cells, FO B cells, and MZ B cells. One of skill in the art would understand that depending on the desired source of the antibody to be obtained in the method described herein, a different source of the B cells may be used for the first sample.

Sequencing Analysis Sample Preparation

In some embodiments, methods provided herein can comprise producing a nucleic acid library comprising a plurality of nucleic acid molecules. In some embodiments, producing a nucleic acid library comprises isolating a plurality of nucleic acids from a host. In some embodiments, a plurality of nucleic acids is a plurality of RNA molecules, e.g., mRNA molecules.

In some embodiments, producing a nucleic acid library comprises producing a cDNA library. In some embodiments, a cDNA library comprises a plurality of cDNA molecules that correspond to a plurality of mRNA molecules isolated from a host. In some embodiments, a plurality of cDNA molecules are double-stranded cDNA molecules.

In various embodiments of the invention, a plurality of nucleic acid sequences that encode a plurality of immunoglobulin variable domains or CDRs are obtained from a sample obtained from an immunized host (i.e., a sample for sequencing or first sample, as described above).

In some embodiments, a plurality of nucleic acid sequences that encode a plurality of immunoglobulin variable domains or CDRs are obtained from said first sample after obtaining a first sample from an immunized host. In some embodiments, a plurality of nucleic acids obtained from the first sample encoding a plurality of immunoglobulin variable domains comprises preparing cDNA from the nucleic acid sequences and sequencing rearranged heavy chain VDJ sequences and/or rearranged light chain VJ sequences in the first sample.

In some embodiments, producing a nucleic acid library comprises enriching for the plurality of nucleic acid molecules. In some embodiments, enriching for a plurality of nucleic acid molecules comprises amplifying the plurality of nucleic acid molecules, e.g., by PCR, e.g., nested PCR. In some embodiments, enriching for a plurality of nucleic acid molecules comprises capturing the plurality of nucleic acid molecules. Capture techniques can include, e.g., hybrid capture techniques.

In some embodiments, methods provided herein comprise attaching an index to each nucleic acid molecule of a nucleic acid library. An index can be sample specific. In some embodiments, an index is between 1-25 nucleotides long. In some embodiments, an index is between 1-10 nucleotides long.

In some embodiments, methods provided herein comprise attaching a sequencing primer and/or its complementary sequence to each nucleic acid molecule of a nucleic acid library.

In some embodiments, a plurality of nucleic acid molecules in a nucleic acid library are fragmented. In some embodiments, nucleic acid molecules are fragmented by mechanical (e.g., sonication) or chemical (e.g., enzymes) methods.

In some embodiments, methods provided herein comprise performing a size-selection on nucleic acid molecules in a nucleic acid library. Size-selection parameters can be determined based on the type of sequencing to be performed. In an exemplary size-selection, nucleic acids are size selected for lengths in the range of 200-1000 bp, e.g., 400-900 bp, e.g., 400-700 bp.

In some embodiments, methods provided herein comprise quantifying the amount of nucleic acid in a nucleic acid library. In some embodiments, an amount can be a total amount, e.g., nanograms of nucleic acid. In some embodiments, an amount can be a concentration, e.g., nanograms of nucleic acid per milliliter.

In some embodiments, a plurality of nucleic acid sequences that encode a plurality of immunoglobulin variable domains is determined using next generation sequencing technology. In some embodiments, a plurality of nucleic acid sequences encode a sufficient number of amino acid sequences for identifying an immunoglobulin variable domain that binds to a particular antigen. Exemplary representative numbers of amino acid sequences can comprise tens, hundreds, thousands, or tens of thousands of sequences. In some embodiments, a final reference sequence database constructed from a plurality of immunoglobulin variable regions determined using next generation sequencing technology will likely exclude single read sequences (e.g., sequences for which only a single sequence read is produced during a sequencing run) in order to reduce impact of sequencing errors. Therefore, in some embodiments, the number of unique amino acid sequences encoded by nucleic acid sequences maybe determined after excluding such single read sequences.

### Next Generation Sequencing (NGS)

Methods provided herein can comprise performing NGS sequencing. In some embodiments, methods provided herein can include performing one or more NGS techniques.

“Next generation sequencing” (NGS), also referred to as massively parallel or deep sequencing, as used herein, relates to sequencing technologies that can sequence millions of small fragments of DNA in parallel and detect variants in the nucleic acid sequence. In some embodiments, nucleic acids are sequenced multiple times in order to provide high fidelity and depth of the results. NGS sequencing can be performed without physical separation of individual reactions. Not wishing to be bound by theory, following nucleic acid extraction, NGS sequencing can be performed using a wide range of instruments and techniques that include targeted sequencing, whole exome sequencing, and whole genome sequencing followed by library or template generation, and data analysis using bioinformatics. Generally, a wide range of platforms and bioinformatics tools exist for performing NGS and data analysis. See e.g. Levy S. E. and Myers R. M., 2016 Annu. Rev. Genom. Hum. Genet. 17: 95-115; Behjati S. and Tarpey P. S., 2013 Arch Dis Child Pract Ed. 98(6): 236-238; Alekseyev, et al. 2018 Academic Pathology 5: 1-11. In some embodiments of methods described herein, deeper sequencing will increase coverage of the antibody repertoire.

Exemplary NGS methods for use in accordance with the present disclosure include sequencing techniques including “second-generation sequencing,” “third-generation sequencing,” and “fourth-generation sequencing” techniques.

In some embodiments, methods provided herein include sequencing by techniques that include, but are not limited to, 454 pyrosequencing, Ion Torrent sequencing, and Illumina sequencing.

In some embodiments, methods provided herein include sequencing by 454 pyrosequencing. 454 pyrosequencing detects pyrophosphate, a byproduct of nucleotide incorporation, to report whether a particular base was incorporated in a growing DNA chain ((Ronaghi, Karamohamed, Pettersson, Uhlen, & Nyren, Anal. Biochem. 1996 Nov. 1; 242(1):84-9.); see also Slatko, Gardner, & Ausubel, Curr. Protoc. Mol. Biol. 2018; 122(1):e59), both of which are incorporated herein by reference in their entirety. In a typical 454 sequencing method, individual DNA fragments, e.g., 400-900 bp, e.g., 400-700 bp long, are ligated to adapters and amplified by PCR in an individual emulsion “bead” (emPCR) reaction. DNA sequences on the beads can be complementary to sequences on the adaptors, allowing the DNA fragments to bind directly to the beads, ideally one fragment to each bead. DNA synthesis followed by chemical detection of the DNA synthesis reactions then occurs and pyrophosphate release is measured. Picoliter-sized chambers including the samples are flooded with sequencing reagents containing one of the 4 nucleotides. When the correct nucleotide is incorporated in the synthesized strand, pyrophosphate release is measured utilizing a light-generating reaction. Homopolymer “runs” of nucleotides in the sequence can be detected by measuring the intensity of the light produced by the reaction. Historically, 454 sequencing technology has been used for genome sequencing and metagenome samples because of the long read lengths (up to 600-800 nt) that are typically achieved and relatively high throughput (25 million bases, at 99% or better accuracy in a 4 hour run), facilitating genome assembly.

In some embodiments, methods provided herein include sequencing by Ion Torrent sequencing. Ion Torrent™ technology directly converts nucleotide sequence into digital information on a semiconductor chip (Rothberg et al., Nature 475, 348-352 (2011), which is incorporated by reference in its entirety). In a DNA synthesis reaction, when a correct nucleotide is incorporated across from its complementary base in a growing DNA chain, a hydrogen ion is released. The release of a hydrogen ion changes the pH of the solution, which can be recorded as a voltage change by an ion sensor, much like a pH meter. If no nucleotide is incorporated, no voltage spike occurs. By sequentially flooding and washing out a “sequencing chamber” with sequencing regents that include only one of the 4 nucleotides at a time, voltage changes occur when the appropriate nucleotide is incorporated. When two adjacent nucleotides incorporate the same nucleotide, two hydrogens are released and the voltage doubles. Thus “runs” of a single nucleotide can also be determined.

Ion Torrent sequencing begins by fragmenting DNA into 200-1500 base fragments, which are ligated to adapters. The DNA fragments are attached to a bead by complementary sequences on the beads and adapters and are then amplified on the bead by emulsion PCR (emPCR). Beads are then flowed across a chip containing wells so that only one bead can enter an individual well. Sequencing reagents are then flowed across the wells, and when the appropriate nucleotide is incorporated, a hydrogen ion is given off and the signal recorded.

In some embodiments, methods provided herein include sequencing by Illumina sequencing. Illumina sequencing is based on a technique known as “bridge amplification” in which DNA molecules (about 500 bp) with appropriate adapters ligated on each end are used as substrates for repeated amplification synthesis reactions on a solid support that contains oligonucleotide sequences complementary to a ligated adapter. Oligonucleotides on the support are spaced such that the DNA, which is then subjected to repeated rounds of amplification, creates clonal “clusters” consisting of about 1000 copies of each oligonucleotide fragment. Each support can include millions of parallel cluster reactions. During the synthesis reactions, modified nucleotides, corresponding to each of the four bases, each with a different fluorescent label, are incorporated and then detected. The nucleotides also act as terminators of synthesis for each reaction, which are unblocked after detection for the next round of synthesis. The reactions are repeated for 300 or more rounds. The use of fluorescent detection increases the speed of detection due to direct imaging, in contrast to camera-based imaging.

In some embodiments, methods provided herein include sequencing by single molecule real time (SMRT) sequencing. SMRT sequencing can enable very long fragments to be sequenced, up to 30-50 kb, or longer. SMRT sequencing involves binding an engineered DNA polymerase, with bound DNA to be sequenced, to the bottom of a well (zero-mode waveguide (ZMW) in a SMRT flow cell. A ZMW is small chamber that guides light energy into an area whose dimensions are small, relative to the wavelength of the illuminating light. Because of the ZMW design and wavelength of light utilized, imaging often occurs only at the bottom of the ZMW where the DNA polymerase, bound to the DNA, incorporates each base in a growing chain. The four nucleotides are labeled with different phospho-linked fluorophores for differential detection. When a nucleotide is incorporated into the growing chain, imaging occurs on the millisecond time scale as the correct fluorescently-labeled nucleotide is bound. After incorporation, the phosphate-linked fluorescent moiety is released and can no longer be detected. The next nucleotide can then be incorporated. Imaging is timed with the rate of nucleotide incorporation so that each base is identified as it is incorporated into the growing DNA chain. This simultaneously occurs in parallel in up to one million zeptoliter ZMWs, present on a single chip within the SMRT cells.

Template preparation with SMRT sequencing involves production of a “SMRTbell,” a circular double-stranded DNA molecule with a known adapter sequence complementary to the primers used to initiate the DNA synthesis on the template. This configuration enables the polymerase to read through large templates numerous times by traversing the circular molecule in each ZMW, until the polymerase stops, to build up a consensus sequence (CC S, circular consensus sequence). As the adapters ligated to each side of the insert each have DNA synthesis priming sites, the sequencing polymerase can traverse the circular SMRTbell in the 5′ to 3′ direction on either DNA strand, providing complementary information from both strands of the ds “SMRTbell”.

In some embodiments, methods provided herein include sequencing by nanopore sequencing. In some embodiments, methods provided herein include sequencing by in situ sequencing (ISS).

### Bioinformatics

In some embodiments, bioinformatics is used to analyze the data produced by sequencing. For example, in some embodiments, bioinformatics can be used to delineate particular regions of an antibody or antigen-binding protein to be analyzed, e.g., a nucleic acid sequence of immunoglobulin variable region, an amino acid sequence of immunoglobulin variable domain, a nucleic acid sequence encoding a framework region or a complementarity determining region, or an amino acid sequence of a framework region or a complementarity determining region.

NGS sequencing typically produces large amounts of sequencing data. In some embodiments, sequence reads can be de-multiplexed. In some embodiments, de-multiplexing comprises in silico sorting of sequence reads based on the sample or source from which the sequenced nucleic acid was obtained. De-multiplexing can be performed by in silico sorting of sequence reads based on an associated index. In some embodiments, after de-multiplexing has been performed, the sequence of the index can be removed from the sequence read. In some embodiments, the identification of the index, source or sample can be added to sequence information associated with the sequence read.

In some embodiments, sequence reads are removed from further analysis (“filtered out”) based on a quality score (e.g., a Phred score). In some embodiments, a quality score represents the probability that one or more nucleotides in a sequence read is called incorrectly. In some embodiments, a quality score is a way to assign confidence to a particular base within a read.

In some embodiments, sequence reads are removed from further analysis (“filtered out”) based on sequence read length. For example, a sequence read that is either too short or too long can be removed from the analysis.

In some embodiments, sequence reads are removed from further analysis (“filtered out”) based on the identity of a portion of the sequence read to a known sequence. For example, in some embodiments, a sequence read can be removed from further analysis if a portion of the sequence read corresponding to a primer (e.g., an IgG constant region primer) has less than 90%, less than 95%, less than 100% identity to the known sequence of the primer.

In some embodiments, sequence reads are removed from further analysis because a low number of reads was detected for a particular nucleic acid sequence.

In some embodiments, nonproductive rearrangements (e.g., those with stop codons or out-of-frame rearrangements) may be removed prior to analysis.

In some embodiments, a method described herein comprises performing NGS that includes performing paired-end sequencing and the method comprises merging overlapping paired-end reads.

In some embodiments, duplicate reads can be removed. Duplicate reads are reads that correspond to the same original DNA fragment. Duplicate reads can be generated, e.g., due to an amplification step in sequencing technique. In some embodiments, removal of duplicate reads occurs prior to determining amino acid sequences encoded by a plurality of nucleic acid sequences in a nucleic acid sequence library.

In some embodiments, sequencing information obtained by performing NGS is used to determine consensus sequences corresponding to the original DNA fragments sequenced.

In some embodiments, nucleotide sequences obtained from the NGS are ranked. In some embodiments, nucleotide sequences are ranked based on cDNA abundance, read length, and/or confidence of the nucleotide sequence. In some embodiments, the top 1,000 sequences of the NGS analysis are ranked. In some embodiments, the top 500 sequences of the NGS analysis are ranked. In some embodiments, the top 400 peptides obtained by MS are ranked. In some embodiments, the top 300 sequences of the NGS analysis are ranked. In some embodiments, the top 200 sequences of the NGS analysis are ranked. In some embodiments, the top 100 sequences of the NGS analysis are ranked.

In some embodiments, the plurality of nucleic acid sequences (e.g., those encoding immunoglobulin variable domains) obtained via NGS is aligned to germline V(D)J sequences. In some embodiments, the plurality of nucleic acid sequences (e.g., those encoding immunoglobulin variable domains) obtained via NGS is aligned to germline V(D)J sequences, and further analyzed to extract information about, e.g., variable region sequences, variable domain sequences, framework sequences, and/or CDR sequences (e.g., CDR3 sequences).

In some embodiments, sequencing reads are analyzed to determine the amino acid sequences they encode (e.g., by in silico translation) and collapsing the sequences into unique full length in frame amino acid sequences. In some embodiments, methods provided comprise generating a library of amino acid sequences by in silico translating sequencing reads, e.g., of the sequence read library.

In some embodiments, the amino acid sequences of these extracted nucleic acid sequences or CDR3 sequences are analyzed to determine their amino acid sequences by obtaining amino acid sequences of the corresponding nucleic acid or CDR3 sequences (e.g., by in silico translation) and collapsing the sequences into unique full length in frame amino acid sequences. In some embodiments, these unique amino acid sequences are used to construct a library of amino acid sequences representing a plurality of immunoglobulin variable domains or immunoglobulin CDRs.

As used herein, nucleic acid sequences that encode a plurality of immunoglobulin variable domains encompass nucleic acid sequences that encode about 10,000-500,000 unique amino acid sequences including about 10,000, about 15,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 95,000, about 100,000, about 110,000, about 120,000, about 130,000, about 140,000, about 150,000, about 160,000, about 170,000, about 180,000, about 190,000, about 200,000, about 250,000, about 300,000, about 350,000, about 400,000, about 450,000, or about 500,000 unique amino acid sequences. In some embodiments, a nucleic acid sequences that encode a plurality of immunoglobulin variable domains may encompass nucleic acid sequences that encode about 10-100,000 unique amino acid sequences, or about 10; about 25; about 50; about 75; about 100; about 250; about 500; about 750; about 1000; about 1500; about 2000; about 2500; about 3000; about 3500; about 4000; about 4500; about 5000; about 10,000; about 15,000; about 20,000; about 25,000; about 30,000; about 35,000; about 40,000; about 45,000; about 50,000; about 55,000; about 60,000; about 65,000; about 70,000; about 75,000; about 80,000; about 85,000; about 90,000; about 95,000; or about 100,000 unique amino acid sequences. In some embodiments, a plurality of nucleic acid sequences encodes about 10,000-80,000 unique amino acid sequences, and may encompass about 10,000; about 15,000; about 20,000; about 25,000; about 30,000; about 35,000; about 40,000; about 45,000; about 50,000; about 55,000; about 60,000; about 65,000; about 70,000; about 75,000; or about 80,000 unique amino acid sequences. Furthermore, in some embodiments, only a single amino acid sequence may be required to identify the immunoglobulin variable domain that binds to a particular antigen.

### Samples for Peptide Analysis

In some embodiments, methods provided herein comprise obtaining and/or determining a plurality of peptide sequences of human immunoglobulin heavy chain and/or light chain variable domains that were obtained from a sample of antibodies. In some embodiments, a sample of antibodies comprises a population of antibodies obtained from an immunized host.

The present disclosure encompasses a recognition that a sample for peptide analysis can be enriched for antibodies with desired characteristics in vivo. For example, a sample of antibodies may be enriched based on in vivo localization. Accordingly, in some embodiments a sample comprising antibodies can be obtained from any desired source within the host, e.g., serum, plasma, lymphoid organs, gut, cerebrospinal fluid, brain, spinal cord, placenta, or a combination thereof.

In some embodiments, a sample for peptide analysis is or comprises any bodily fluid comprising antibodies. In some embodiments, a sample for peptide analysis is or comprises a sample obtained from serum, plasma, lymphoid organs, gut, cerebrospinal fluid, brain, spinal cord, placenta, or a combination thereof. In some certain embodiments, a sample for peptide analysis is or comprises antibodies obtained from serum of an immunized host (e.g., non-human animal, e.g., rodent). In some embodiments, a sample for peptide analysis (a “second sample”) can be obtained from a tissue lysate. In some embodiments, second samples may contain varying levels of circulating antibodies that can be isolated and sequenced. As described above, in some embodiments, the second sample may be derived from a particular antibody source, e.g., secreted antibody source, if evaluation of antibody from that source is desired. In some embodiments, a sample for peptide analysis comprises antibodies obtained from a particular tissue to enrich for antibodies that localize to that tissue.

In some embodiments, a sample for peptide analysis comprises a population of antibodies. In some embodiments, a sample for peptide analysis is enriched for antibodies with desired characteristics ex vivo. In some embodiments, a sample is enriched for antibodies using chromatography, such as, for example, ion exchange chromatography. In some embodiments, a sample is enriched for antibodies with affinity to a particular target using, e.g., affinity chromatography. In some embodiments, affinity chromatography is used to remove antibodies with certain undesired (e.g., off target) binding affinities. In some embodiments, a sample for peptide analysis is enriched for antibodies with desired characteristics by exposing the antibody to one or more conditions, e.g., heat and/or oxidation to select for antibody stability.

In some embodiments, a second sample comprises antibodies directed against the antigen of interest from the immunized host, and is depleted of antibodies not directed against the antigen of interest. The depletion of samples can be achieved using a variety of methods including, but not limited to chromatography, affinity purification methods, size exclusion methods, buffer exchanges, albumin depletion techniques, protease inhibitors, immunoglobulin depletion techniques, and high abundant protein depletion. In some embodiments, where the immunogen during immunization of the non-human animal is complexed with an adjuvant, the second sample maybe depleted of antibodies directed against the adjuvant. In some embodiments, wherein the immunogen is fused to an Fc moiety, the second sample is depleted of antibodies directed against the Fc. In other embodiments, the immunogen may be fused to a tag, e.g., His, FLAG, Myc, HA, GST, GFP, V5, etc., and the second sample depleted of antibodies directed against that tag.

In some embodiments, the second sample is enriched for antibodies directed against the antigen of interest. Similar to depletion methods, the enrichment of samples can be achieved using a variety of methods including chromatography, affinity purification methods, size exclusion methods, etc. In some embodiments, the second sample may be enriched by various methods that involve binding to the antigen immunogen. Since the enrichment step may depend on antibody binding to a polypeptide; in this step, an antibody pool can be interrogated for a specific property of the antibody of interest. In one example, the second sample may be enriched for an antibody of interest based on its ability to bind to an antigen under specific binding conditions. For example, the second sample may be enriched for antibody of interest based on its ability bind to a specific isoform/variant of the antigen, specific fragment/epitope of the antigen, monomeric or oligomeric forms of the antigen, or other desired conformations of the antigen. In some embodiments, a sample for peptide analysis is enriched for a particular Ig class, for example, by affinity chromatography using protein A (or anti-IgA and anti-IgM antibodies for affinity purification of the other major Ig classes).

In some embodiments, a sample comprising a population of antibodies is digested and/or fragmented prior to peptide analysis. In some embodiments, a sample of antibodies for peptide analysis is digested into peptides. In some embodiments, a sample of antibodies for peptide analysis is enzymatically digested into peptides (e.g., using trypsin and/or pepsin). In some embodiments, a sample of antibodies for peptide analysis is denatured and reduced prior to digestion. In some embodiments, a sample of antibodies for peptide analysis is alkylated (e.g, using iodoacetamide) prior to digestion. In some embodiments, a sample of antibodies for peptide analysis is denatured, reduced and/or alkylated and then enzymatically digested (e.g., using trypsin and/or pepsin). In some embodiments, a sample is divided into multiple aliquots that are digested with different enzymes and/or for different amounts of time. In some embodiments, a sample is divided into at least two aliquots that are digested with at least 2 different enzymes.

In some embodiments, antibodies are digested into peptides and sequenced using MS analysis (e.g., tandem mass spectrometry). In some embodiments, peptide sequences from MS analysis are interrogated against of a library of antibody sequences.

In some embodiments, peptides of antibody are separated and/or resolved by chromatography, e.g., liquid chromatography. In some embodiments, peptides of antibody are separated and/or resolved by high performance liquid chromatography. In some embodiments, peptides of antibody are separated and/or resolved by reverse phase chromatography.

In certain embodiments, CDR3 peptides could be enriched from unrelated peptides via specific conjugation of the unique Cys at the end of the CDR3 sequence with a thiol-specific reagent that allows the purification of such peptides. In some embodiments, a sample of antibodies for peptide analysis is digested (e.g., enzymatically digested) into a plurality of peptides and the plurality of peptides are enriched for CDR3 peptides using a thiol-specific reagent.

### MS and Interrogation of the Library

In some embodiments, methods described herein utilize mass spectrometry (MS). Mass spectrometry obtains molecular weight and structural information on chemical compounds by ionizing the molecules and measuring either their time-of-flight or the response of the molecular trajectories to electric and/or magnetic fields.

The present disclosure further contemplates that any MS method can be adapted for use in methods of the disclosure. Exemplary MS methods include, but are not limited to, tandem MS (MS/MS), LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Such methods are described in, e.g., Pitt, Clin. Biochem. Rev. 30:19-34 (2009). Mass spectrometers that can be used in methods of the present disclosure are known in the art and are commercially available from, e.g., Agilent Inc., Bruker Corporation, and Thermo Scientific.

In some embodiments, the peptide sequences of a second sample are determined using mass spectrometric analysis of the heavy and/or light chain variable domains of the population of antibodies. In some embodiments, the mass spectrometric analysis combines liquid chromatography and mass spectrometry (LC-MS) preceded by a proteolytic digest of the heavy and/or light chain variable domains of the population of antibodies. However, alternative separation and mass spectrometry methods can be used including accelerator mass spectrometry, gas chromatography-mass spec (GC-MS), ion mobility spectrometry-MS, Matrix Assisted Laser Desorption Ionization Time of Flight (MALDI-TOF), and Surface Enhanced Laser Desorption Ionization (SELDI-TOF). In general, top-down proteomics can also be used wherein intact proteins are analyzed without digestion thereby retaining intact protein mass information. See Chen et al. 2018 Anal Chem. 90(1): 110-127. In some embodiments, provided methods incorporate multidimensional high-pressure liquid chromatography (LC/LC) and/or tandem mass spectrometry (MS/MS).

In some certain embodiments, a MS analysis is quantitative.

In some embodiments, peptide sequences obtained from the MS analysis are ranked. In some embodiments, peptide sequences are ranked based on peptide abundance and/or peptide confidence. In some embodiments, the top 1,000 peptides obtained by MS are ranked. In some embodiments, the top 500 peptides obtained by MS are ranked. In some embodiments, the top 400 peptides obtained by MS are ranked. In some embodiments, the top 300 peptides obtained by MS are ranked. In some embodiments, the top 200 peptides obtained by MS are ranked. In some embodiments, the top 100 peptides obtained by MS are ranked. In some embodiments, the MS spectra quality of the top ranked peptide sequences is manually confirmed.

In various embodiments, the peptide sequences (e.g., the peptide sequences of heavy and/or light chain variable domains) obtained through MS analysis (e.g., of the second sample) are interrogated with amino acid sequences of the plurality of immunoglobulin variable domains obtained from the sequence analysis (e.g., of a first sample). In some embodiments, the peptide sequences are interrogated with amino acid sequences obtained by translation of nucleotide sequences obtained by NGS (e.g., of a first sample).

In some embodiments, interrogating the amino acids sequences of the plurality of immunoglobulin variable domains with the peptide sequences of heavy and/or light chain variable domains of the population of antibodies comprises aligning the peptide sequences of heavy and/or light chain variable domains of the population of antibodies to each other and to the amino acid sequences of the plurality of immunoglobulin variable domains. Aligning, as used herein, also means comparing the peptide sequences of heavy and/or light chain variable domains of the population of antibodies to the amino acid sequences of the plurality of immunoglobulin variable domains and, optionally, to each other. The peptide sequences obtained through mass spectrometric analysis of the second sample may, in some embodiments, be screened against the library containing the plurality of variable domains obtained from the first sample. As contemplated by the present disclosure, interrogating the amino acid sequence can be performed using a variety of methods.

In some embodiments, peptide sequences obtained through mass spectrometric analysis of a second sample are mapped and/or searched against a library of antibody sequences (e.g., variable domain sequences and/or CDR sequences) obtained from the sequencing analysis (e.g., of a first sample) using commercially available software (e.g., Mascot, Martix Science; PEAKS, Bioinformatics Solutions, Inc.; Sequest, ThermoFisher Scientific; Byonic, Protein Metrics). Based on the various criteria, the sequence of the variable domain of the antibody of interest is obtained.

In some embodiments, obtaining a human immunoglobulin heavy chain and/or light chain variable domain or a CDR of an antibody specific for the antigen is based on one or more of: (1) a match (e.g., specified homology) of a unique peptide obtained from the second sample to a CDR3 sequence in the amino acid sequence obtained from the first sample; (2) a match (e.g., specified homology) of unique peptides obtained from the second sample to CDR1 and/or CDR2 sequences in the amino acid sequence obtained from the first sample; (3) a match (e.g., specified homology) of one or more unique peptides obtained from the second sample to one or more framework sequences in the amino acid sequence obtained from the first sample; (4) the number of next generation sequencing counts, (5) exclusion of CDR sequence with methionine; and (6) exclusion of CDR sequence with potential N glycosylation. In some embodiments, obtaining a human immunoglobulin heavy chain and/or light chain variable domain or a CDR of an antibody specific for the antigen is based on combination of two or more, three or more, four or more, five or more, or all six of these parameters.

In some embodiments, obtaining a human immunoglobulin heavy chain variable domain or a CDR of an antibody specific for the antigen is based on homology of a unique peptide obtained from MS analysis to CDR sequences and/or framework sequences in the library. In some embodiments, the library comprises amino acid sequences of antibody heavy chain variable domains that correspond to nucleic acid sequences obtained by NGS (e.g., of a first sample obtained from an immunized host).

In some embodiments, peptide sequences obtained from MS analysis are used to interrogate the library to select for only those amino acid sequences that share at least 90%, at least 91%, at least 92%, at least 93%, at least 94%, at least 95%, at least 96%, at least 97%, at least 98%, at least 99% identity or 100% identity.

In some embodiments, interrogation comprises querying a library for sequences homologous to peptide sequences (e.g., CDR sequences) obtained through MS analysis. In some embodiments, interrogation comprises querying a library for sequences homologous to CDR3 peptide sequences obtained through MS analysis. In some embodiments, interrogation comprises querying a library for sequences that are at least 90%, 91%, 92%, 93%, 94%, 95%, 96%, 97%, 98%, or 99% homologous to CDR3 peptide sequences obtained through MS analysis. In some embodiments, interrogation comprises querying a library for sequences that are 100% homologous to CDR3 peptide sequences obtained through MS analysis.

In some embodiments, peptide sequences obtained through MS analysis of the second sample are searched against a library of antibody sequences (e.g., variable domain sequences and/or CDR sequences) obtained from the sequencing analysis, using one or more of the following search paramaters: enzymatic cleavage site, enzymatic digestion specificity, missed enzymatic cleavages, mass tolerance, and/or fixed modifications. In some embodiments, peptide sequences corresponding to a CDR (e.g., CDR3) of an antibody variable domain obtained through MS of a sample are mapped and/or searched against a library of antibody sequences (e.g., CDRs sequences) obtained from the sequencing analysis (e.g., of a first sample) using commercially available software.

In various embodiments of the present invention, a match of a peptide obtained from mass spectrometry analysis of the second sample to the library of amino acid sequences generated through NGS includes peptides that are 80% or greater identical to the NGS-obtained sequence. In some embodiments, the percent identity of the peptide obtained from mass spectrometry analysis of the second sample to the library of amino acid sequences generated through NGS is at least about 80%, at least about 90%, at least about 95%, at least about 96%, at least about 97%, at least about 98%, or at least about 99% identical to the NGS-obtained sequence. The term “identity” as used herein, in connection with alignment or comparison of the peptide sequence to the NGS-obtained sequence, refers to identity as determined by a number of different algorithms known in the art used to measure nucleotide and/or amino acid sequence identity. In a further embodiment, a match can be an exact match of the peptide sequence to the NGS-obtained sequence. In some embodiments, a peptide obtained from MS analysis may cover the entire CDR or framework sequence or a portion thereof in the NGS database.

In some embodiments, an obtained antibody, variable domain, and/or CDR sequences are selected based on one or more criteria. In some embodiments, antibody sequences (or portions thereof) are grouped based on homology. In some embodiments, obtained antibody and/or variable domain sequences are grouped based on homology of one or more CDRs. In some embodiments, obtained antibody and/or variable domain sequences are grouped based on CDR3 homology.

In some embodiments, immunoglobulin heavy chain variable domain sequences are grouped based on homology. In some embodiments, immunoglobulin light chain variable domain sequences are grouped based on homology.

In some embodiments, peptide sequences mapped onto the library of antibody sequences (e.g., variable domain sequences and/or CDR sequences) obtained from the sequencing analysis are ranked. In some embodiments, peptide sequences are ranked based on sequence coverage and/or peptide confidence. In some embodiments, the top 1,000 antibody hits are ranked. In some embodiments, the top 500 antibody hits are ranked. In some embodiments, the top 400 antibody hits are ranked. In some embodiments, the top 300 antibody hits are ranked. In some embodiments, the top 200 antibody hits are ranked. In some embodiments, the top 100 antibody hits are ranked. In some embodiments, the MS spectra quality of the top ranked peptide sequences is manually confirmed.

In some embodiments, identified immunoglobulin heavy chain and/or light chain variable domain sequences are expressed as a recombinant antigen-binding protein (e.g., antibody). In some embodiments, identified immunoglobulin heavy chain and/or light chain variable domain sequences are codon optimized and expressed as a recombinant antigen-binding protein.

In some embodiments, recombinant antigen-binding proteins (e.g., antibodies) comprising identified variable domain sequences are characterized. In some embodiments, binding affinity for a target is assessed for recombinant antibodies comprising identified variable domain sequences.

### Non-Human Animals

Methods provided herein include the use of non-human animals. Exemplary non-human animals for use with the discloses methods are described in detail below. Briefly, however, in various embodiments, the host (e.g., the immunized host) is a genetically modified non-human animal, e.g., non-human mammal, that comprises in its genome an immunoglobulin heavy chain variable region comprising one or more human heavy chain V gene segments, one or more human D gene segments, and one or more human heavy chain J gene segments, wherein the heavy chain variable region is operably linked to a constant region, and an immunoglobulin light chain variable region comprising one or more human light chain V gene segments and one or more human light chain J gene segments, wherein the light chain is operably linked to a constant region.

In some embodiments, the genetically modified non-human animal can be any non-human animal. In some embodiments, the non-human animal is a vertebrate. In some embodiments, the non-human animal is a mammal. In some embodiments, the genetically modified non-human animal described herein may be selected from a group consisting of a mouse, rat, rabbit, pig, bovine (e.g., cow, bull, buffalo), deer, sheep, goat, llama, chicken, cat, dog, ferret, primate (e.g., marmoset, rhesus monkey). For non-human animals where suitable genetically modifiable ES cells are not readily available, other methods can be employed to make a non-human animal comprising the genetic modifications described herein. Such methods include, for example, modifying a non-ES cell genome (e.g., a fibroblast or an induced pluripotent cell) and employing nuclear transfer to transfer the modified genome to a suitable cell, such as an oocyte, and gestating the modified cell (e.g., the modified oocyte) in a non-human animal under suitable conditions to form an embryo.

In some embodiments, the non-human animal is a mammal. In some embodiments, the non-human animal is a small mammal, e.g., of the superfamily Dipodoidea or Muroidea. In some embodiments, the non-human animal is a rodent. In certain embodiments, the rodent is a mouse, a rat or a hamster. In some embodiments, the rodent is selected from the superfamily Muroidea. In some embodiments, the non-human animal is from a family selected from Calomyscidae (e.g., mouse-like hamsters), Cricetidae (e.g., hamster, New World rats and mice, voles), Muridae (e.g., true mice and rats, gerbils, spiny mice, crested rats), Nesomyidae (e.g., climbing mice, rock mice, white-tailed rats, Malagasy rats and mice), Platacanthomyidae (e.g., spiny dormice), and Spalacidae (e.g., mole rates, bamboo rats, and zokors). In some embodiments, the rodent is selected from a true mouse or rat (family Muridae), a gerbil, a spiny mouse, and a crested rat. In some embodiments, the mouse is from a member of the family Muridae. In some embodiments, the non-human animal is a rodent. In some embodiments, the rodent is selected from a mouse and a rat. In some embodiments, the non-human animal is a mouse.

In some embodiments, the non-human animal is a mouse of a C57BL strain. In some embodiments, the C57BL strain is selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/01a. In some embodiments, the non-human animal is a mouse of a 129 strain. In some embodiments, the 129 strain is selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. In some embodiments, the genetically modified mouse is a mix of a 129 strain and a C57BL strain. In some embodiments, the mouse is a mix of 129 strains and/or a mix of C57BL/6 strains. In some embodiments, the 129 strain of the mix is a 129S6 (129/SvEvTac) strain. In some embodiments, the mouse is a BALB strain (e.g., BALB/c). In some embodiments, the mouse is a mix of a BALB strain and another strain (e.g., a C57BL strain and/or a 129 strain). In some embodiments, the non-human animals provided herein can be a mouse derived from any combination of the aforementioned strains.

In some embodiments, the non-human animal provided herein is a rat. In some embodiments, the rat is selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

Thus, in some embodiments the immunized non-human animal host is a rodent such as a rat or mouse. Thus, in some embodiments, the host is a genetically modified rodent that comprises in its genome an immunoglobulin heavy chain variable region comprising one or more human heavy chain V gene segments (also referred to as human V_{H} gene segments), one or more human D gene segments (also referred to as human D_{H} gene segments), and one or more human heavy chain J gene segments (also referred to as human J_{H} gene segments), wherein the heavy chain variable region is operably linked to a constant region, and an immunoglobulin light chain variable region comprising one or more human light chain V gene segments and one or more human light chain J gene segments, wherein the light chain is operably linked to a constant region.

In some embodiments, the host is a genetically modified mouse that comprises in its genome an immunoglobulin heavy chain variable region comprising one or more human heavy chain V gene segment, one or more human D gene segments, and one or more human heavy chain J gene segments, wherein the heavy chain variable region is operably linked to a murine constant region, and an immunoglobulin light chain variable region comprising one or more human light chain V gene segments and one or more human light chain J gene segments, wherein the light chain is operably linked to a murine constant region.

In one aspect, the immunoglobulin heavy chain variable region comprising human heavy chain V, D, and J gene segments is operably linked to a mouse heavy chain constant region, and the immunoglobulin light chain variable region comprising human light chain V and J gene segments is operably linked to a mouse light chain constant region. In a further aspect the immunoglobulin heavy chain variable region comprising human heavy chain V, D, and J gene segments operably linked to a mouse heavy chain constant region resides at the endogenous mouse heavy chain locus, and the immunoglobulin light chain variable region comprising human light chain V and J gene segments operably linked to a mouse light chain constant region resides at the endogenous mouse light chain locus. Various embodiments of the genetically modified non-human animals, e.g., rodents, e.g., mice, are described in more detail herein below.

In some embodiments, the host is a genetically modified non-human animal comprising a restricted heavy or restricted light chain variable sequence, e.g., comprising a limited repertoire of heavy or light chain variable V(D)J gene segments, e.g., single rearranged heavy or light chain variable sequence, as described herein below.

### Genetically Modified Hosts for Identification of Antigen-Specific Antibodies

The antibodies of the present invention are obtained by first immunizing the non-human animal host with an antigen of interest. Thus, in some embodiments, an immunized non-human animal host as described herein is a rodent, e.g., a rat or mouse. In some embodiments, an immunized non-human animal host as described herein is a genetically modified non-human animal host, e.g., a genetically modified rodent. Various embodiments of the genetically modified non-human animals, e.g., rodents, e.g., rats or mice, are described in more detail herein below.

In some embodiments, the immunized non-human animal host is a rodent such as a rat or mouse. In some embodiments, the host is a genetically modified rodent that comprises in its genome an immunoglobulin heavy chain variable region comprising one or more human heavy chain V gene segments, one or more human D gene segments, and one or more human heavy chain J gene segments, wherein the immunoglobulin heavy chain variable region is operably linked to a constant region, and an immunoglobulin light chain variable region comprising one or more human light chain V gene segments and one or more human light chain J gene segments, wherein the light chain is operably linked to a constant region.

In some embodiments, the host is a genetically modified mouse that comprises in its genome an immunoglobulin heavy chain variable region comprising one or more human heavy chain V gene segments, one or more human D gene segments, and one or more human heavy chain J gene segments, wherein the heavy chain variable region is operably linked to a murine (e.g., a rat or mouse) constant region, and an immunoglobulin light chain variable region comprising one or more human light chain V gene segments and one or more human light chain J gene segments, wherein the light chain is operably linked to a murine constant region.

In some embodiments, the immunoglobulin heavy chain variable region is operably linked to a mouse heavy chain constant region, and the immunoglobulin light chain variable region is operably linked to a mouse light chain constant region. In some embodiments, the immunoglobulin heavy chain variable region operably linked to a mouse heavy chain constant region resides at the endogenous mouse heavy chain locus, and the immunoglobulin light chain variable region operably linked to a mouse light chain constant region resides at the endogenous mouse light chain locus. One exemplary embodiment is described in Macdonald et al., Proc. Natl. Acad. Sci. USA 111:5147-52 and supporting information (www.pnas.org/cgi/content/short/1323896111), which is hereby incorporated by reference in its entirety. Various embodiments of the genetically modified non-human animals, e.g., rodents, e.g., rats or mice, are described in more detail herein below.

In some embodiments, a genetically modified rodent comprises in its genome (e.g., its germline genome) an engineered immunoglobulin heavy chain locus (e.g., an engineered endogenous rodent immunoglobulin heavy chain locus) comprising one or more unrearranged human V_{H} gene segments, one or more unrearranged human D_{H} gene segments, and one or more unrearranged human J_{H} gene segments that are upstream of (e.g., operably linked to) one or more rodent (e.g., rat or mouse) immunoglobulin heavy chain constant region genes (e.g., one or more endogenous rodent (e.g., rat or mouse) immunoglobulin heavy chain constant region genes). Such an engineered immunoglobulin heavy chain locus is referred to herein as an “HoH locus.” Rodents including an HoH locus are exemplified in, e.g., U.S. Pat. Nos. 6,596,541; 8,642,835; and 8,697,940, and Murphy, A., “VelocImmune: Immunoglobulin Variable Region Humanized Mouse,” in Recombinant Antibodies for Immunotherapy, New York, N.Y., Cambridge University Press, 101-107 (2009), each of which is incorporated by reference in its entirety. In some embodiments, a genetically modified rodent (e.g., rat or mouse) is homozygous at an HoH locus. In some embodiments, a genetically modified rodent (e.g., rat or mouse) is heterozygous at an HoH locus.

In some embodiments, one or more unrearranged human V_{H} gene segments includes at least six human V_{H} gene segments. In some embodiments, one or more unrearranged human V_{H} gene segments includes at least 18 human V_{H} gene segments. In some embodiments, one or more unrearranged human V_{H} gene segments includes at least 39 human V_{H} gene segments. In some embodiments, one or more unrearranged human V_{H} gene segments includes at least 80 human V_{H} gene segments. In some embodiments, one or more unrearranged human D_{H} gene segments includes at least 27 human D_{H} gene segments. In some embodiments, one or more unrearranged human J_{H} gene segments includes at least six human J_{H} gene segments.

In some embodiments, one or more unrearranged human V_{H} gene segments includes all functional human V_{H} gene segments. In some embodiments, one or more unrearranged human V_{H} gene segments includes less than 80 human V_{H} gene segments. In some embodiments, one or more unrearranged human V_{H} gene segments includes less than 39 human V_{H} gene segments. In some embodiments, one or more unrearranged human V_{H} gene segments includes less than 18 human V_{H} gene segments. In some embodiments, one or more unrearranged human V_{H} gene segments includes less than 10 human V_{H} gene segments.

In some embodiments, one or more unrearranged human V_{H} gene segments includes at least 18 human V_{H} gene segments, one or more unrearranged human D_{H} gene segments includes 27 human D_{H} gene segments, and one or more unrearranged human V_{H} gene segments includes six human J_{H} gene segments. Such an engineered immunoglobulin heavy chain locus is referred to herein as a “VelocImmune® 1 HoH locus.” In some embodiments, one or more unrearranged human V_{H} gene segments includes at least 39 human V_{H} gene segments, one or more unrearranged human D_{H} gene segments includes 27 human D_{H} gene segments, and one or more unrearranged human J_{H} gene segments includes six human J_{H} gene segments. Such an engineered immunoglobulin heavy chain locus is referred to herein as a “VelocImmune® 2 HoH locus.” In some embodiments, one or more unrearranged human V_{H} gene segments includes at least 80 human V_{H} gene segments, one or more unrearranged human D_{H} gene segments includes 27 human D_{H} gene segments, and one or more unrearranged human J_{H} gene segments includes six human J_{H} gene segments. Such an engineered immunoglobulin heavy chain locus is referred to herein as a “VelocImmune® 3 HoH locus.”

In some embodiments, a genetically modified rodent (e.g., rat or mouse), which comprises an HoH locus, produces an antibody comprising, inter alia, heavy chains, wherein each heavy chain comprises a human heavy chain variable domain operably linked to a rodent (e.g., rat or mouse) heavy chain constant domain, e.g., in response to antigenic stimulation.

In some embodiments, a genetically modified rodent comprises in its genome (e.g., its germline genome) an engineered immunoglobulin heavy chain locus (e.g., an engineered endogenous rodent immunoglobulin heavy chain locus) comprising one or more unrearranged human V_{H} gene segments, one or more unrearranged human D_{H} gene segments, and one or more unrearranged human J_{H} gene segments, which further comprises substitution or insertion of at least one histidine for a non-histidine residue, such that the unrearranged immunoglobulin heavy chain variable gene sequence comprises in a complementarity determining region 3 (CDR3) encoding sequence a substitution of at least one non histidine codon with a histidine codon or an insertion of at least one histidine codon (see, e.g., PCT Pub. Nos. WO2013/138712 and WO2013/138681, incorporated herein by reference in their entireties). Immunizing genetically modified rodents comprising substitution of non-histidine residues with histidine residues or insertion of histidine residues facilitates identification of antibodies that exhibit pH-dependent properties towards their antigens, using the combination of repertoire sequencing and MS methods described herein and in the Examples.

In some embodiments, a genetically modified rodent (e.g., rat or mouse) comprises in its genome (e.g., its germline genome) an engineered immunoglobulin heavy chain locus, such as comprising a restricted heavy chain variable region sequence, comprising a limited human heavy chain variable region repertoire.

In some embodiments, a genetically modified rodent comprises in its genome (e.g., its germline genome) an engineered immunoglobulin heavy chain locus (e.g., an engineered endogenous rodent immunoglobulin heavy chain locus) comprising a single human V_{H} gene segment, one or more unrearranged human D_{H} gene segments, and one or more unrearranged human J_{H} gene segments that are upstream of (e.g., operably linked to) one or more rodent (e.g., rat or mouse) immunoglobulin heavy chain constant region genes (e.g., one or more endogenous rodent (e.g., rat or mouse) immunoglobulin heavy chain constant region genes). A genetically modified rodent having such an engineered immunoglobulin heavy chain locus (e.g., an engineered endogenous rodent immunoglobulin heavy chain locus) is exemplified in, e.g., U.S. Patent Publication No. 2019/0261612 and U.S. Pat. No. 10,238,093, each of which is incorporated by reference in its entirety.

In some embodiments, a genetically modified rodent (e.g., rat or mouse) comprises in its genome (e.g., its germline genome) an engineered immunoglobulin heavy chain locus (e.g., an engineered endogenous rodent immunoglobulin heavy chain locus) comprising a single rearranged human heavy chain variable region upstream of (e.g., operably linked to) one or more rodent (e.g., rat or mouse) constant region genes. Such an engineered immunoglobulin heavy chain locus is referred to herein as a “UHC locus” or a “universal heavy chain locus” or a “common heavy chain locus.” Rodents including a UHC locus are exemplified in, e.g., U.S. Pat. No. 9,204,624, which is incorporated by reference in its entirety.

In some embodiments, a single rearranged human heavy chain variable region comprises a single human V_{H} gene segment, a single human D_{H} gene segment, and a single human V_{H} gene segment. In some embodiments, a single human V_{H} gene segment is a human V_{H}3-23, a single human D_{H} gene segment is a human D_{H}4-4, and a single human J_{H} gene segment is a human J_{H}4.

In some embodiments, a single rearranged human heavy chain variable region comprises a single human V_{H} gene segment and a single human J_{H} gene segment, which are separated by two amino acids. In some embodiments, a single human V_{H} gene segment is a human V_{H}3-23, a single human J_{H} gene segment is a human J_{H}4, and two amino acids are glycine and tyrosine.

In some embodiments, one or more rodent (e.g., mouse or rat) heavy chain constant region genes are one or more endogenous rodent (e.g., mouse or rat) heavy chain constant region genes.

In some embodiments, a genetically modified rodent (e.g., rat or mouse), which comprises a UHC locus, produces an antibody comprising, inter alia, immunoglobulin chains, where each immunoglobulin chain comprises a human heavy chain variable domain operably linked to a constant domain, e.g., in response to antigenic stimulation.

In some embodiments, a genetically modified rodent (e.g., rat or mouse) comprises in its genome (e.g., its germline genome) an engineered immunoglobulin heavy chain locus (e.g., an engineered endogenous rodent immunoglobulin heavy chain locus) comprising one or more unrearranged human V_{L} gene segments and one or more unrearranged human J_{L}, gene segments that are upstream of (e.g., operably linked to) one or more rodent (e.g., rat or mouse) immunoglobulin heavy chain constant region genes (e.g., one or more endogenous rodent (e.g., rat or mouse) immunoglobulin heavy chain constant region genes). In some embodiments, such a genetically modified rodent comprises a hybrid heavy chain locus with both light chain (e.g., light chain variable region) and heavy chain (e.g., heavy chain constant region) sequences. Such an engineered immunoglobulin heavy chain locus is referred to herein as an “LoH locus.” Rodents including an LoH locus are exemplified in, e.g., U.S. Pat. No. 9,686,970 and U.S. Patent Publication No. 2013/0212719, each of which is incorporated by reference in its entirety. In some embodiments, one or more unrearranged human V_{L} gene segments and one or more unrearranged human J_{L} gene segments are one or more unrearranged human Vκ gene segments and one or more unrearranged human Jκ gene segments. In some embodiments, one or more unrearranged human V_{L} gene segments and one or more unrearranged human J_{L} gene segments are one or more unrearranged human Vλ gene segments and one or more unrearranged human Jλ gene segments. In some embodiments, a genetically modified rodent (e.g., rat or mouse) is homozygous at an LoH locus. In some embodiments, a genetically modified rodent (e.g., rat or mouse) is heterozygous at an LoH locus.

In some embodiments, a genetically modified rodent (e.g., rat or mouse), which comprises an LoH locus, produces an antibody comprising, inter alia, immunoglobulin chains, where each immunoglobulin chain comprises a human light chain variable domain operably linked to a rodent (e.g., rat or mouse) heavy chain constant domain, e.g., in response to antigenic stimulation.

In some embodiments, the immunized rodent produces antibodies comprising two immunoglobulin heavy chains and two immunoglobulin light chains. In some embodiments, the immunized rodent does not produce single domain antibodies, heavy chain only antibodies, and/or nanobodies.

In some embodiments, a genetically modified rodent (e.g., rat or mouse) as provided herein has a genome (e.g., a germline genome) comprising a modification including a deletion of a nucleic acid sequence encoding a CH1 domain of an endogenous IgG constant region gene, referred to herein as a “CH1 delete modification.” In some embodiments, a genetically modified rodent (e.g., rat or mouse), which comprises a CH1 delete modification, produces an IgG heavy chain antibody comprising, inter alia, immunoglobulin heavy chains, where each immunoglobulin heavy chain lacks a CH1 domain, in whole or in part. In some embodiments, a genetically modified rodent (e.g., rat or mouse) as provided herein has a genome (e.g., a germline genome) comprising a heavy chain only immunoglobulin encoding sequence comprising an unrearranged human heavy chain variable region in operable linkage to an endogenous heavy chain constant region, wherein the endogenous heavy chain constant region comprises (1) an intact endogenous IgM gene that encodes an IgM isotype that associates with light chain and (2) a non-IgM gene, e.g., an IgG gene, lacking a sequence that encodes a functional CH1 domain, wherein the non-IgM gene encodes a non-IgM isotype lacking a CH1 domain capable of covalently associating with a light chain constant domain. In some embodiments, an IgG antibody produced also lacks a cognate light chain and secretes an IgG heavy chain only antibody into its serum. Exemplary rodents comprising a CH1 delete modification are described, e.g., in U.S. Pat. No. 8,754,287, US Patent Publication. No. 2015/0289489, and PCT Pub. Nos. WO2006/008548, WO2010/109165, and WO2016062990, each incorporated herein by reference in its entirety. In some embodiments, the immunized rodent produces single domain antibodies, a heavy chain only antibodies, and/or nanobodies.

In some embodiments, the present disclosure provides methods of identifying a human immunoglobulin heavy chain variable domain or CDR sequence (e.g., CDR3 sequence) of an antibody specific for an antigen from a rodent comprising in its germline genome heavy chain immunoglobulin variable region comprising a CH1 deletion modification, the method comprising: (i) obtaining a plurality of peptide sequences of human immunoglobulin heavy chain variable domains that were obtained from a sample comprising a population of antibodies produced by a genetically modified rodent immunized with the antigen, and (ii) interrogating a library of human immunoglobulin heavy chain variable domain sequences with the plurality of peptide sequences, wherein the library comprises a plurality of human immunoglobulin heavy chain variable domain sequences encoded by B cells of the immunized rodent.

In some embodiments, the present disclosure provides methods of identifying a human immunoglobulin heavy chain variable domain or CDR sequence (e.g., CDR3 sequence) of an antibody specific for an antigen from a rodent comprising in its germline genome heavy chain immunoglobulin variable region comprising a CH1 deletion modification, the method comprising: (i) obtaining a library of human immunoglobulin heavy chain variable domain sequences comprising a plurality of human immunoglobulin heavy chain variable domain sequences encoded by B cells of a rodent immunized with the antigen, and (ii) interrogating the library with a plurality of peptide sequences of human immunoglobulin heavy chain variable domains that were obtained from a sample comprising a population of antibodies produced by the rodent immunized with the antigen.

In some embodiments, a genetically modified rodent (e.g., rat or mouse) as provided herein has a genome (e.g., a germline genome) comprising an engineered immunoglobulin heavy chain (e.g., HoH, UHC, LoH) locus (e.g., an engineered endogenous rodent immunoglobulin heavy chain locus) lacking a functional endogenous rodent Adam6 gene. In some embodiments, a genetically modified rodent (e.g., rat or mouse) as provided herein has a genome (e.g., a germline genome) comprising one or more nucleotide sequences encoding one or more rodent ADAM6 polypeptides, functional orthologs, functional homologs, or functional fragments thereof. In some embodiments, one or more rodent ADAM6 polypeptides is or comprises mouse ADAM6a. In some embodiments, one or more rodent ADAM6 polypeptides is or comprises mouse ADAM6b. In some embodiments, one or more rodent ADAM6 polypeptides is or comprises mouse ADAM6a and mouse ADAM6b. Rodents including one or more nucleotide sequences encoding one or more rodent ADAM6 polypeptides, functional orthologs, functional homologs, or functional fragments thereof are exemplified in, e.g., U.S. Pat. Nos. 8,642,835; 8,697,940; 9,706,759; 10,130,081; 10,238,093, and U.S. Patent Publication No. 2013/0212719, each of which is incorporated by reference in its entirety. In some embodiments, a genetically modified rodent (e.g., rat or mouse) as provided expresses one or more rodent (e.g., rat or mouse) ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof that are included on the same chromosome as an engineered immunoglobulin heavy chain (e.g., HoH, UHC, LoH) locus. In some embodiments, a genetically modified rodent (e.g., rat or mouse) as provided has a genome (e.g., a germline genome) comprising an engineered immunoglobulin heavy chain (e.g., HoH, UHC, LoH) locus comprising one or more nucleotide sequences encoding one or more rodent ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof in place of a human Adam6 pseudogene. In some embodiments, a genetically modified rodent (e.g., rat or mouse) as provided has a genome (e.g., germline genome) comprising one or more nucleotide sequences encoding one or more rodent (e.g., rat or mouse) ADAM6 polypeptides, functional orthologs, functional homologs, or functional fragments thereof that replace a human Adam6 pseudogene.

In some embodiments, a genetically modified rodent as provided has a genome (e.g., a germline genome) comprising one or more human V_{H} gene segments comprising a first and a second human V_{H} gene segment, and one or more nucleotide sequences encoding one or more rodent (e.g., rat or mouse) ADAM6 polypeptides, functional orthologs, functional homologs, or functional fragments thereof between the first human V_{H} gene segment and the second human V_{H} gene segment. In some embodiments, a first human V_{H} gene segment is V_{H}1-2 and a second human V_{H} gene segment is V_{H}6-1.

In some embodiments, one or more nucleotide sequences encoding one or more rodent (e.g., a rat or mouse) ADAM6 polypeptides, functional orthologs, functional homologs, or functional fragments thereof are between a human V_{H} gene segment and a human D_{H} gene segment.

In some embodiments, one or more nucleotide sequences encoding one or more rodent ADAM6 polypeptides restore or enhance fertility in a male rodent.

In some embodiments, a genetically modified rodent (e.g., rat or mouse) comprises in its genome (e.g., its germline genome) an engineered immunoglobulin light chain locus (e.g., an engineered endogenous rodent immunoglobulin light chain locus) comprising one or more unrearranged human V_{L} gene segments and one or more unrearranged human J_{L} gene segments that are upstream of (e.g., operably linked to) one or more immunoglobulin light chain constant region genes. In some embodiments, one or more unrearranged human V_{L} gene segments and one or more unrearranged human J_{L} gene segments are one or more unrearranged human Vκ gene segments and one or more unrearranged human Jκ gene segments. In some embodiments, one or more unrearranged human V_{L} gene segments and one or more unrearranged human J_{L} gene segments are one or more unrearranged human Vλ gene segments and one or more unrearranged human Jλ gene segments. In some embodiments, one or more unrearranged immunoglobulin light chain constant region genes is or comprises a Cκ. In some embodiments, one or more unrearranged immunoglobulin light chain constant region genes is or comprises a Cλ.

In some embodiments, an engineered immunoglobulin light chain locus (e.g., an engineered endogenous rodent immunoglobulin light chain locus) comprises a non-native leader sequence. In some embodiments, a leader sequence comprises a signal peptide. In some embodiments, a leader sequence comprises a non-native signal peptide.

In some embodiments, a genetically modified rodent (e.g., rat or mouse) comprises in its genome (e.g., its germline genome) an engineered immunoglobulin light chain locus (e.g., an engineered endogenous rodent immunoglobulin light chain locus) comprising one or more unrearranged human Vκ gene segments and one or more unrearranged human Jκ gene segments that are upstream of (e.g., operably linked to) a Cκ gene. Such an engineered immunoglobulin light chain locus is referred to herein as a “KoK locus.” Rodents including a KoK locus are exemplified in, e.g., U.S. Pat. Nos. 6,596,541; 8,642,835; and 8,697,940, each of which is incorporated by reference in its entirety. In some embodiments, an immunoglobulin κ light chain constant region gene of a KoK locus is a rodent (e.g., rat or mouse) Cκ gene. In some embodiments, an immunoglobulin κ light chain constant region gene of a KoK locus is an endogenous rodent (e.g., rat or mouse) Cκ gene. In some embodiments, an immunoglobulin κ light chain constant region gene of a KoK locus is an endogenous rodent (e.g., rat or mouse) Cκ gene at an endogenous immunoglobulin κ light chain locus. In some embodiments, a genetically modified rodent (e.g., rat or mouse) is homozygous at a KoK locus. In some embodiments, a genetically modified rodent (e.g., rat or mouse) is heterozygous at a KoK locus.

In some embodiments, a genetically modified rodent (e.g., rat or mouse), which comprises a KoK locus, produces an antibody comprising, inter alia, κ light chains, where each κ light chain comprises a human κ light chain variable domain operably linked to a rodent (e.g., rat or mouse) κ light chain constant domain, e.g., in response to antigenic stimulation.

In some embodiments, one or more unrearranged human Vκ gene segments includes at least six human Vκ gene segments. In some embodiments, one or more unrearranged human Vκ gene segments includes at least 16 human Vκ gene segments. In some embodiments, one or more unrearranged human Vκ gene segments includes at least 30 human Vκ gene segments. In some embodiments, one or more unrearranged human Vκ gene segments includes at least 40 human Vκ gene segments. In some embodiments, one or more unrearranged human Jκ gene segments includes at least five human Jκ gene segments.

In some embodiments, one or more unrearranged human Vκ gene segments includes at least 16 human Vκ gene segments, and one or more unrearranged human Jκ gene segments includes at least five human Jκ gene segments. Such an engineered immunoglobulin light chain locus is referred to herein as a “VelocImmune® 1 KoK locus.” In some embodiments, one or more unrearranged human Vκ gene segments includes at least 30 human Vκ gene segments, and one or more unrearranged human Jκ gene segments includes at least five human Jκ gene segments. Such an engineered immunoglobulin light chain locus is referred to herein as a “VelocImmune® 2 KoK locus.” In some embodiments, one or more unrearranged human Vκ gene segments includes at least 40 human Vκ gene segments, and one or more unrearranged human Jκ gene segments includes at least five human Jκ gene segments. Such an engineered immunoglobulin light chain locus is referred to herein as a “VelocImmune® 3 KoK locus.”

In some embodiments, a genetically modified rodent (e.g., rat or mouse) comprises in its genome (e.g., its germline genome) an engineered immunoglobulin light chain locus (e.g., an engineered endogenous rodent immunoglobulin light chain locus) comprising one or more unrearranged human Vλ gene segments upstream of (e.g., operably linked to) one or more unrearranged human Jλ gene segments and one or more Cλ genes. Such an engineered immunoglobulin light chain locus is referred to herein as an “LoL locus.” Mice including an LoL locus are exemplified in, e.g., U.S. Pat. Nos. 9,012,717; 9,226,484; 9,029,628, and U.S. Patent Publication No. 2018/0125043, each of which is incorporated by reference in its entirety. In some embodiments, the one or more unrearranged human Jλ gene segments and one or more Cλ genes of an LoL locus are present in Jλ-Cλ clusters. In some embodiments, one or more Cλ genes of an LoL locus comprise one or more human Cλ genes. In some embodiments, one or more Cλ genes of an LoL locus comprise one or more mouse Cλ genes. In some embodiments, one or more Cλ genes of an LoL locus comprise one or more human Cλ genes and one or more mouse Cλ genes. In some embodiments, one or more mouse Cλ genes of an LoL locus comprise a mouse Cλ1 gene. In some embodiments, a genetically modified rodent (e.g., rat or mouse) is homozygous at an LoL locus. In some embodiments, a genetically modified rodent (e.g., rat or mouse) is heterozygous at an LoL locus.

In some embodiments, a genetically modified rodent (e.g., rat or mouse), which comprises an LoL locus, produces an antibody comprising, inter alia, λ light chains, where each λ light chain comprises a human λ light chain variable domain operably linked to a rodent (e.g., rat or mouse) λ light chain constant domain, e.g., in response to antigenic stimulation. In some embodiments, a genetically modified rodent (e.g., rat or mouse), which comprises an LoL locus, produces an antibody comprising, inter alia, λ light chains, where each λ light chain comprises a human λ light chain variable domain operably linked to a human λ light chain constant domain, e.g., in response to antigenic stimulation.

In some embodiments, a genetically modified rodent (e.g., rat or mouse) comprises in its genome (e.g., its germline genome) an engineered immunoglobulin light chain locus comprising one or more unrearranged human Vλ gene segments and one or more unrearranged human gene segments upstream of (e.g., operably linked to) a Cκ gene. Such an engineered immunoglobulin light chain locus is referred to herein as an “LoK locus.” Rodents including an LoK locus are exemplified in, e.g., U.S. Pat. Nos. 9,006,511 and 9,035,128, each of which is incorporated by reference in its entirety. In some embodiments, a Cκ gene of an LoK locus is a rodent (e.g., rat or mouse) Cκ gene. In some embodiments, a Cκ gene of an LoK locus is an endogenous rodent (e.g., rat or mouse) Cκ gene. In some embodiments, a Cκ gene of an LoK locus is an endogenous rodent (e.g., rat or mouse) Cκ gene at an endogenous immunoglobulin light chain locus. In some embodiments, a genetically modified rodent (e.g., rat or mouse) is homozygous at an LoK locus. In some embodiments, a genetically modified rodent (e.g., rat or mouse) is heterozygous at an LoK locus.

In some embodiments, a genetically modified rodent (e.g., rat or mouse), which comprises an LoK locus, produces an antibody comprising, inter alia, light chains, where each light chain comprises a human λ light chain variable domain operably linked to a rodent (e.g., rat or mouse) κ light chain constant domain, e.g., in response to antigenic stimulation.

In some embodiments, a genetically modified rodent (e.g., rat or mouse) comprises in its genome (e.g., its germline genome) an engineered immunoglobulin κ light chain locus (e.g., an engineered endogenous rodent immunoglobulin κ light chain locus) comprising one or more unrearranged human Vλ gene segments and one or more unrearranged human Jλ gene segments upstream of (e.g., operably linked to) a Cλ gene. Such an engineered immunoglobulin light chain locus is referred to herein as an “LiK locus.” Rodents including an LiK locus are exemplified in, e.g., U.S. Patent Publication No. 2019/0223418 (issued as U.S. Pat. No. 11,051,498), which is incorporated by reference in its entirety. In some embodiments, a Cλ gene of an LiK locus is a rodent (e.g., rat or mouse) Cλ gene. In some embodiments, a Cλ gene of an LiK locus is a mouse Cλ1 gene. In some embodiments, a genetically modified rodent (e.g., rat or mouse) is homozygous at an LiK locus. In some embodiments, a genetically modified rodent (e.g., rat or mouse) is heterozygous at an LiK locus.

In some embodiments, a genetically modified rodent (e.g., rat or mouse), which comprises an LiK locus, produces an antibody comprising, inter alia, λ light chains, where each λ light chain comprises a human λ light chain variable domain operably linked to a rodent (e.g., rat or mouse) λ light chain constant domain, e.g., in response to antigenic stimulation.

In some embodiments, a genetically modified rodent (e.g., rat or mouse) comprises in its genome (e.g., its germline genome) an engineered immunoglobulin κ light chain locus (e.g., an engineered endogenous rodent immunoglobulin κ light chain locus) comprising one or more unrearranged human Vλ gene segments upstream of (e.g., operably linked to) one or more unrearranged human gene segments and one or more human Cλ genes. In some embodiments, the one or more unrearranged human gene segments and one or more Cλ genes of such an engineered immunoglobulin κ light chain locus are present in Jλ-Cλ clusters. In some embodiments, a genetically modified rodent (e.g., rat or mouse) is homozygous for such an engineered immunoglobulin κ light chain locus. In some embodiments, a genetically modified rodent (e.g., rat or mouse) is heterozygous for such an engineered immunoglobulin κ light chain locus. In some embodiments, a genetically modified rodent (e.g., rat or mouse), which comprises such an engineered immunoglobulin κ light chain locus, produces an antibody comprising, inter alia, λ light chains, where each λ light chain comprises a human λ light chain variable domain operably linked to a human λ light chain constant domain, e.g., in response to antigenic stimulation.

In some embodiments, a genetically modified rodent (e.g., rat or mouse) has a germline genome comprising a limited human light chain variable region repertoire.

Exemplary genetically modified rodents, comprising human V(D)J gene segments having a germline genome comprising a limited human light chain variable region repertoire are described in, e.g., U.S. Pat. Nos. 9,796,788; 10,130,081; 10,143,186; 10,167,344; 10,412,940; and 10,130,081; as well as WO 2019/008123, WO2020/247623, and WO2020/132557, each of which is hereby incorporated by reference in its entirety. In some embodiments, a limited human light chain variable region repertoire comprises a limited number of human V_{L} gene segments. In some embodiments, a limited number of human V_{L} gene segments comprises two human V_{L} gene segments. In some embodiments, a limited number of human V_{L} gene segments is one human V_{L} gene segment. For example, in some embodiments a limited number of human V_{L} gene segments is one human Vκ gene segment. One human Vκ gene segment can be, e.g., a human Vκ 1-39 gene segment, a human Vκ3-15 gene segment, a human Vκ3-11 gene segment, or a human Vκ3-20 gene segment. In some embodiments a limited number of human V_{L} gene segments is one human Vλ gene segment. One human Vλ gene segment can be, e.g., a human Vλ1-51 gene segment, a human Vλ5-45 gene segment, a human Vλ1-44 gene segment, a human Vλ1-40 gene segment, a human Vλ3-21 gene segment, or a human Vλ2-14 gene segment.

In some embodiments, a limited human light chain variable region repertoire comprises one or more J_{L} gene segments. In some embodiments, a limited human light chain variable region repertoire comprises one J_{L} gene segment. In some embodiments, one J_{L} gene segment is a Jκ gene segment. In some embodiments, one J_{L} gene segment is a Jλ gene segment. In some embodiments, one J_{L} gene segment is a human J_{L} gene segment. In some embodiments, one J_{L} gene segment is a mouse J_{L} gene segment.

In some embodiments, a limited human light chain variable region repertoire comprises (i) a human Vκ gene segment and a human Jκ gene segment, (ii) a human Vκ gene segment and a mouse Jκ gene segment, (iii) a human Vκ gene segment and a human Jλ gene segment, or (iv) a human Vκ gene segment and a mouse Jλ gene segment.

In some embodiments, a limited human light chain variable region repertoire comprises (i) a human Vλ gene segment and a human gene segment, (ii) a human Vλ gene segment and a mouse gene segment, (iii) a human Vλ gene segment and a human Jκ gene segment, or (iv) a human Vλ gene segment and a mouse Jκ gene segment.

In some embodiments, a limited human light chain variable region repertoire comprises (i) a human Vκ1-39 gene segment and a human Jκ5 gene segment, (ii) a human W1-39 gene segment and a human Jκ1 gene segment, (iii) a human Vκ 3-20 gene segment and a human Jκ1 gene segment, or (iv) a human Vκ 3-20 gene segment and a human Jκ5 gene segment.

In some embodiments, a limited human light chain variable region repertoire comprises (i) a human Vκ1-39 gene segment and a mouse Jκ2 gene segment, (ii) a human Vκ 3-20 gene segment and a mouse Jκ2 gene segment, or (iii) a human W3-15 gene segment and a mouse Jκ2 gene segment.

In some embodiments, a limited human light chain variable region repertoire comprises (i) a human Vλ1-51 gene segment and a human Jλ2 gene segment, (ii) a human Vλ5-45 gene segment and a human Jλ2 gene segment, (iii) a human Vλ1-44 gene segment and a human Jλ2 gene segment, (iv) a human Vλ1-40 gene segment and a human Jλ2 gene segment, (v) a human Vλ3-21 gene segment and a human Jλ2 gene segment, or (vi) a human Vλ2-14 gene segment and a human Jλ2 gene segment.

In some embodiments, a limited human light chain variable region repertoire is operably linked to a Cκ gene segment. In some embodiments, a Cκ gene segment is human. In some embodiments, a Cκ gene segment is mouse. In some embodiments, a mouse Cκ gene segment is an endogenous mouse Cκ gene segment, e.g., at an endogenous mouse immunoglobulin κ light chain locus. In some embodiments, a mouse Cκ gene segment is at an endogenous mouse immunoglobulin λ light chain locus.

In some embodiments, a limited human light chain variable region repertoire is operably linked to a Cλ gene segment. In some embodiments, a Cλ gene segment is human. In some embodiments, a Cλ gene segment is mouse. In some embodiments, a mouse Cλ gene segment is an endogenous mouse Cλ gene segment, e.g., at an endogenous mouse immunoglobulin λ light chain locus. In some embodiments, a mouse Cλ gene segment is at an endogenous mouse immunoglobulin κ light chain locus.

In some embodiments, a genetically modified mouse is heterozygous for a limited human light chain variable region repertoire. In some embodiments, a genetically modified mouse is homozygous for a limited human light chain variable region repertoire.

In some embodiments, a genetically modified rodent comprises an engineered immunoglobulin light chain locus (e.g., an engineered endogenous rodent immunoglobulin light chain locus) comprising a restricted light chain variable region sequence, comprising a limited human light chain variable region repertoire. In some embodiments, a limited human light chain variable region repertoire comprises one or two human light chain V gene segments and one or more human light chain J gene segments. In some embodiments, a limited human light chain variable region repertoire is operably linked to a light chain constant region gene segment. In some embodiments, a genetically modified rodent (e.g., rat or mouse) comprising a limited human light chain variable region repertoire comprises in its genome (e.g., its germline genome) exactly two unrearranged human light chain V gene segments and one or more unrearranged human light chain J gene segments operably linked to a light chain constant region sequence. Such an engineered immunoglobulin light chain locus is referred to herein as a “DLC locus.” In some embodiments, a genetically modified rodent comprising a limited human light chain variable region repertoire comprises in its genome (e.g., its germline genome) a single rearranged light chain variable region locus comprising a single human light chain V gene segment rearranged to a single human light chain J gene segment. In some embodiments, a genetically modified rodent (e.g., rat or mouse) comprising a limited human light chain variable region repertoire comprises in its genome (e.g., its germline genome) a single rearranged light chain variable region locus operably linked to a light chain constant region sequence, where the single rearranged light chain variable region locus comprises a single human light chain V gene segment rearranged to a single human light chain J gene segment. Such an engineered immunoglobulin light chain locus is referred to herein as “ULC locus.” As used herein, the phrase “ULC locus” is interchangeable with “universal light chain locus” or “common light chain locus”.

In some embodiments, a genetically modified rodent (e.g., rat or mouse) has a germline genome comprising a limited human κ light chain variable region repertoire. In some embodiments, a genetically modified rodent comprises an engineered immunoglobulin κ light chain locus (e.g., an engineered endogenous rodent immunoglobulin κ light chain locus) comprising a limited human κ light chain variable region repertoire. In some embodiments, a limited human κ light chain variable region repertoire comprises one or two human Vκ gene segments and one or more human Jκ gene segments. In some embodiments, a limited human κ light chain variable region repertoire is operably linked to a light chain constant region gene segment. In some embodiments, a genetically modified rodent as provided comprises a limited human κ light chain variable region repertoire operably linked to a Cκ gene segment.

In some embodiments, a genetically modified rodent (e.g., rat or mouse) comprises in its genome (e.g., its germline genome) a limited human κ light chain variable region repertoire, wherein the limited human κ light chain variable region repertoire comprises a single rearranged human κ light chain variable region (Vκ/Jκ). A single rearranged human κ light chain variable region comprises a human Vκ gene segment joined to a human Jκ gene segment. In some embodiments, a genetically modified rodent (e.g., rat or mouse) comprises in its genome (e.g., its germline genome) an engineered immunoglobulin κ light chain locus (e.g., an engineered endogenous rodent immunoglobulin κ light chain locus) comprising a single rearranged human κ light chain variable region upstream of (e.g., operably linked to) a Cκ gene. Such an engineered immunoglobulin light chain locus is referred to as a “κULC locus” and is an example of a ULC locus. Rodents including a κULC locus are exemplified in, e.g., U.S. Pat. Nos. 10,130,081 and 10,143,186, each of which is incorporated by reference in its entirety.

In some embodiments, a single rearranged human κ light chain variable region comprises a human Vκ gene segment and a human Jκ gene segment. In some embodiments, a human Vκ gene segment is a human Vκ1-39 gene segment or a human Vκ3-20 gene segment. In some embodiments, a human Jκ gene segment is a human Jκ1 gene segment, a human Jκ2 gene segment, a human Jκ3 gene segment, a human Jκ4 gene segment, or a human Jκ5 gene segment. In some embodiments, a human Vκ gene segment is a human Vκ1-39 gene segment, and a human Jκ gene segment is a human Jκ5 gene segment. In some embodiments, a single rearranged human κ light chain variable region is a human Vκ1-39/Jκ5. In some embodiments, a human Vκ gene segment is a human Vκ3-20 gene segment, and a human Jκ gene segment is a human Jκ1 gene segment. In some embodiments, a single rearranged human κ light chain variable region is a human Vκ3-20/Jκ1. In some embodiments, a human Vκ gene segment is a human Vκ 3-11 gene segment, and a human Jκ gene segment selected from a human Jκ1 gene segment, a human Jκ2 gene segment, a human Jκ3 gene segment, a human Jκ4 gene segment, or a human Jκ5 gene segment. In some embodiments, a human Vκ gene segment is a human Vκ 3-11 gene segment, and a human Jκ gene segment is human Jκ1 gene segment. In some embodiments, a single rearranged human κ light chain variable region is a Vκ3-11/Jκ1.

In some embodiments, a Cκ gene of a κULC locus is a rodent (e.g., rat or mouse) Cκ gene. In some embodiments, a genetically modified rodent (e.g., rat or mouse) is homozygous at a κULC locus. In some embodiments, a genetically modified rodent (e.g., rat or mouse) is heterozygous at a κULC locus.

In some embodiments, a genetically modified rodent (e.g., rat or mouse), which comprises a κULC locus, lacks endogenous Vκ and/or Jκ gene segments that are capable of rearranging to form an endogenous κ light chain variable region. In some embodiments, a genetically modified rodent (e.g., rat or mouse), which comprises a κULC locus, lacks endogenous Vλ and/or Jλ gene segments that are capable of rearranging to form an endogenous λ light chain variable region.

In some embodiments, a genetically modified rodent (e.g., rat or mouse), which comprises a κULC locus, produces an antibody comprising, inter alia, κ light chains, where each κ light chain comprises a human κ light chain variable domain operably linked to a rodent (e.g., rat or mouse) κ light chain constant domain, e.g., in response to antigenic stimulation. In some embodiments, all κ light chains expressed by B cells of a genetically modified rodent (e.g., rat or mouse), which comprises a κULC locus, comprise human κ light chain variable domains expressed from the single rearranged human κ light chain variable region or a somatically hypermutated version thereof.

In some embodiments, a genetically modified rodent (e.g., rat or mouse) comprises in its genome (e.g., its germline genome) an engineered immunoglobulin κ light chain locus (e.g., an engineered endogenous rodent immunoglobulin κ light chain locus) comprising exactly two unrearranged human Vκ gene segments and one or more unrearranged human Jκ gene segments operably linked to a κ light chain constant region sequence of (e.g., operably linked to) a Cκ gene. Such an engineered immunoglobulin κ light chain locus is referred to herein as a “κDLC locus,” and is an example of a DLC locus. Rodents including a κDLC locus are exemplified in, e.g., U.S. Pat. Nos. 9,796,788; 10,167,344; 10,412,940; and 10,130,081, each of which is incorporated by reference in its entirety.

In some embodiments, exactly two unrearranged human Vκ gene segments comprise a human Vκ1-39 gene segment and a human Vκ 3-20 gene segment. In some embodiments, one or more unrearranged human Jκ gene segments comprises two human Jκ gene segments. In some embodiments, one or more unrearranged human Jκ gene segments comprises three human Jκ gene segments. In some embodiments, one or more unrearranged human Jκ gene segments comprises four human Jκ gene segments. In some embodiments, one or more unrearranged human Jκ gene segments comprises five human Jκ gene segments. In some embodiments, one or more unrearranged human Jκ gene segments comprises a human Jκ1 gene segment, a human Jκ2 gene segment, a human Jκ3 gene segment, a human Jκ4 gene segment, a human Jκ5 gene segment, or a combination thereof.

In some embodiments, a genetically modified rodent (e.g., rat or mouse), which comprises a κDLC locus, comprises in its genome (e.g., germline genome) exactly two unrearranged human Vκ gene segments and five unrearranged human Jκ gene segments. In some embodiments, exactly two unrearranged human Vκ gene segments comprises a human Vκ1-39 gene segment and a human Vκ 3-20 gene segment, and five unrearranged human Jκ gene segments comprise a human Jκ1 gene segment, a human Jκ2 gene segment, a human Jκ3 gene segment, a human Jκ4 gene segment, and a human Jκ5 gene segment.

In some embodiments, a Cκ gene of a κDLC locus is a rodent (e.g., rat or mouse) Cκ gene. In some embodiments, a genetically modified rodent (e.g., rat or mouse) is homozygous at a κDLC locus. In some embodiments, a genetically modified rodent (e.g., rat or mouse) is heterozygous at a κDLC locus.

In some embodiments, a genetically modified rodent (e.g., rat or mouse), which comprises a κDLC locus, lacks endogenous immunoglobulin Vκ and/or Jκ gene segments that are capable of rearranging to form an endogenous immunoglobulin κ light chain variable region. In some embodiments, a genetically modified rodent (e.g., rat or mouse), which comprises a κDLC locus, lacks endogenous Vλ and/or Jλ gene segments that are capable of rearranging to form an endogenous λ light chain variable region.

In some embodiments, a genetically modified rodent (e.g., rat or mouse), which comprises a κDLC locus, produces an antibody comprising, inter alia, κ light chains, where each κ light chain comprises a human κ light chain variable domain operably linked to a rodent (e.g., rat or mouse) κ light chain constant domain, e.g., in response to antigenic stimulation.

In some embodiments, a genetically modified rodent (e.g., rat or mouse) has a genome (e.g., germline genome) comprising a limited human λ light chain variable region repertoire. In some embodiments, a genetically modified rodent (e.g., rat or mouse) has a genome (e.g., germline genome) comprising an engineered immunoglobulin κ light chain locus (e.g., an engineered endogenous rodent immunoglobulin κ light chain locus) comprising a limited human λ light chain variable region repertoire. In some embodiments, a genetically modified rodent (e.g., rat or mouse) comprises an engineered immunoglobulin κ light chain locus (e.g., an engineered endogenous rodent immunoglobulin κ light chain locus) comprising a limited human λ light chain variable region repertoire, wherein the limited human λ light chain variable region repertoire comprises one or two human Vλ gene segments and one or more human gene segments. In some embodiments, a genetically modified rodent (e.g., rat or mouse) comprises a limited human λ light chain variable region repertoire operably linked to a light chain constant region gene segment. In some embodiments, a genetically modified rodent (e.g., rat or mouse) as provided comprises a limited human λ light chain variable region repertoire operably linked to a rodent (e.g., rat or mouse) Cκ gene segment. In some embodiments, a genetically modified rodent as provided comprises a limited human λ light chain variable region repertoire operably linked to a rodent (e.g., rat or mouse) Cλ gene segment.

In some embodiments, a genetically modified rodent (e.g., rat or mouse) has a genome (e.g., germline genome) comprising an engineered immunoglobulin κ light chain locus (e.g., an engineered endogenous rodent immunoglobulin κ light chain locus) that comprises a limited human λ light chain variable region repertoire, wherein the limited human λ light chain variable region repertoire comprises a single rearranged human immunoglobulin λ light chain variable region (Vλ/Jλ). A single rearranged human λ light chain variable region comprises a human Vλ gene segment joined to a human gene segment. In some embodiments, a genetically modified rodent comprises a limited human λ light chain variable region repertoire operably linked to a rodent (e.g., rat or mouse) Cκ or Cλ gene segment (e.g., a mouse Cλ 1 gene segment). Such an engineered immunoglobulin light chain locus is an example of a ULC locus and is referred to herein as a “ULCiK locus.” Rodents including a ULCiK locus are exemplified in, e.g., WO2020/247623, which is incorporated by reference in its entirety.

In some embodiments, a human Vλ gene segment is selected from a group consisting of: Vλ4-69, Vλ8-61, Vλ4-60, Vλ6-57, Vλ10-54, Vλ5-52, Vλ1-51, Vλ9-49, Vλ1-47, Vλ7-46, Vλ5-45, Vλ1-44, Vλ7-43, Vλ1-40, Vλ5-37, Vλ1-36, Vλ3-27, Vλ3-25, Vλ2-23, Vλ3-22, Vλ3-21, Vλ3-19, Vλ2-18, Vλ3-16, Vλ2-14, Vλ3-12, Vλ2-11, Vλ3-10, Vλ3-9, Vλ2-8, Vλ4-3, and Vλ3-1. In some embodiments, a human Vλ gene segment is selected from a group consisting of: Vλ5-52, Vλ1-51, Vλ9-49, Vλ1-47, Vλ7-46, Vλ5-45, Vλ1-44, Vλ7-43, Vλ1-40, Vλ5-37, Vλ1-36, Vλ3-27, Vλ3-25, Vλ2-23, Vλ3-22, Vλ3-21, Vλ3-19, Vλ2-18, Vλ3-16, Vλ2-14, Vλ3-12, Vλ2-11, Vλ3-10, Vλ3-9, Vλ2-8, Vλ4-3, and Vλ3-1. In some embodiments, a human Vλ gene segment is selected from a group consisting of: Vλ1-51, Vλ5-45, Vλ1-44, Vλ1-40, Vλ3-21, and Vλ2-14. In some embodiments, a human Vλ gene segment is Vλ1-51 or Vλ2-14. In some embodiments, a human gene segment is selected from a group consisting of: Jλ1, Jλ2, Jλ3, Jλ6, and Jλ7. In some embodiments, a human gene segment is selected from a group consisting of: Jλ1, Jλ2, Jλ3, and Jλ7. In some embodiments, a human gene segment is Jλ2.

In some embodiments, a genetically modified rodent (e.g., rat or mouse), which comprises a ULCiK locus, lacks endogenous Vκ and/or Jκ gene segments that are capable of rearranging to form an endogenous κ light chain variable region. In some embodiments, a genetically modified rodent (e.g., rat or mouse), which comprises a ULCiK locus, lacks endogenous Vλ and/or Jλ gene segments that are capable of rearranging to form an endogenous λ light chain variable region.

In some embodiments, a genetically modified rodent (e.g., rat or mouse), which comprises a ULCiK locus, produces an antibody comprising, inter alia, light chains, wherein each light chain comprises a human λ light chain variable domain operably linked to a (e.g., rat or mouse) light chain constant domain (e.g., a Cλ, or Cκ domain), e.g., in response to antigenic stimulation. In some embodiments, all light chains expressed by B cells of a genetically modified rodent (e.g., rat or mouse), which comprises a ULCiK locus, comprise human λ light chain variable domains expressed from the single rearranged human λ light chain variable region or a somatically hypermutated version thereof.

In some embodiments, a genetically modified rodent (e.g., rat or mouse) has a genome (e.g., germline genome) comprising an engineered immunoglobulin κ light chain locus (e.g., an engineered endogenous rodent immunoglobulin κ light chain locus) that comprises a limited human λ light chain variable region repertoire, wherein the limited human λ light chain variable region repertoire comprises two unrearranged human Vλ gene segments and one or more unrearranged human Jλ gene segments. In some embodiments, a limited human λ light chain variable region repertoire comprises two unrearranged human Vλ gene segments and four unrearranged human Jλ gene segments. In some embodiments, a limited human λ light chain variable region repertoire comprises two unrearranged human Vλ gene segments and five unrearranged human Jλ gene segments. In some embodiments, a genetically modified rodent comprises a limited human λ light chain variable region repertoire operably linked to a rodent (e.g., rat or mouse) Cλ gene segment (e.g., a mouse Cλ 1 gene segment). Such an engineered immunoglobulin light chain locus is an example of a DLC locus and is referred to herein as a “DLCiK locus.” Rodents including a DLCiK locus are exemplified in, e.g., WO2020/247623, which is incorporated by reference in its entirety.

In some embodiments, a germline genome of the genetically modified rodent is homozygous for a engineered immunoglobulin κ light chain locus comprising a limited human λ light chain variable region repertoire. In some embodiments, a germline genome of the genetically modified rodent is heterozygous for a engineered immunoglobulin κ light chain locus comprising a limited human λ light chain variable region repertoire.

In some embodiments, a genetically modified rodent (e.g., rat or mouse), which comprises a DLCiK locus, lacks endogenous immunoglobulin Vκ and/or Jκ gene segments that are capable of rearranging to form an endogenous immunoglobulin κ light chain variable region. In some embodiments, a genetically modified rodent (e.g., rat or mouse), which comprises a DLCiK locus, lacks endogenous Vλ and/or Jλ gene segments that are capable of rearranging to form an endogenous λ light chain variable region.

In some embodiments, a genetically modified rodent (e.g., rat or mouse), which comprises a DLCiK locus, produces an antibody comprising, inter alia, light chains, where each light chain comprises a human λ light chain variable domain operably linked to a rodent (e.g., rat or mouse) light chain constant domain (e.g., a Cλ or Cκ domain), e.g., in response to antigenic stimulation.

In some embodiments, a genetically modified rodent (e.g., rat or mouse) comprises an exogenous terminal deoxynucleotidyl transferase (TdT) gene. Rodents including an exogenous TdT are exemplified in, e.g., U.S. Patent Publication No. 2019/0223418 and PCT Publication No. WO 2017/210586, each of which is incorporated by reference in its entirety. In some embodiments, a rodent (e.g., rat or mouse) that comprises an exogenous TdT gene can have increased antigen receptor diversity when compared to a rodent without an exogenous TdT gene.

In some embodiments, a rodent as described herein has a genome comprising an exogenous TdT gene operably linked to a transcriptional control element.

In some embodiments, a transcriptional control element includes a RAG1 transcriptional control element, a RAG2 transcriptional control element, an immunoglobulin heavy chain transcriptional control element, an immunoglobulin κ light chain transcriptional control element, an immunoglobulin λ light chain transcriptional control element, or any combination thereof.

In some embodiments, an exogenous TdT is located at an immunoglobulin κ light chain locus, an immunoglobulin λ light chain locus, an immunoglobulin heavy chain locus, a RAG1 locus, or a RAG2 locus.

In some embodiments, a TdT is a human TdT. In some embodiments, a TdT is a short isoform of TdT (TdTS).

In some embodiments, a genetically modified rodent (e.g., rat or mouse) comprises in its genome (e.g., its germline genome) a HoH locus and a KoK locus. In some embodiments, a genetically modified rodent (e.g., rat or mouse) comprises in its genome (e.g., its germline genome) a HoH locus and a LoL locus. In some embodiments, a genetically modified rodent (e.g., rat or mouse) comprises in its genome (e.g., its germline genome) a HoH locus, a KoK locus, and a LoL locus. In some embodiments, a genetically modified rodent (e.g., rat or mouse) is homozygous at a HoH locus, a KoK locus, a LoL locus, or a combination thereof.

In some embodiments, a genetically modified rodent (e.g., rat or mouse) comprises in its genome (e.g., its germline genome) a HoH locus, a KoK locus, and a LoK locus. In some embodiments, a genetically modified rodent (e.g., rat or mouse) comprises in its genome (e.g., its germline genome) a HoH locus, a KoK locus, and a LiK locus.

In some embodiments, a genetically modified rodent (e.g., rat or mouse) comprises in its genome (e.g., its germline genome) a HoH locus and a LoK locus. In some embodiments, a genetically modified rodent (e.g., rat or mouse) is homozygous at a HoH locus, a LoK locus, or a combination thereof.

In some embodiments, a genetically modified rodent (e.g., rat or mouse) comprises in its genome (e.g., its germline genome) a HoH locus and a LiK locus. In some embodiments, a genetically modified rodent (e.g., rat or mouse) is homozygous at a HoH locus, a LiK locus, or a combination thereof.

In some embodiments, a genetically modified rodent (e.g., rat or mouse) comprises in its genome (e.g., its germline genome) a HoH locus and a ULC locus. In some embodiments, a genetically modified rodent (e.g., rat or mouse) is homozygous at a HoH locus, a ULC locus, or a combination thereof.

In some embodiments, a genetically modified rodent (e.g., rat or mouse) comprises in its genome (e.g., its germline genome) a HoH locus and a DLC locus. In some embodiments, a genetically modified rodent (e.g., rat or mouse) is homozygous at a HoH locus, a DLC locus, or a combination thereof.

In some embodiments, a genetically modified rodent (e.g., rat or mouse) comprises in its genome (e.g., its germline genome) a HoH locus and a κULC locus. In some embodiments, a genetically modified rodent (e.g., rat or mouse) is homozygous at a HoH locus, a κULC locus, or a combination thereof.

In some embodiments, a genetically modified rodent (e.g., rat or mouse) comprises in its genome (e.g., its germline genome) a HoH locus and a κDLC locus. In some embodiments, a genetically modified rodent (e.g., rat or mouse) is homozygous at a HoH locus, a κDLC locus, or a combination thereof.

In some embodiments, a genetically modified rodent (e.g., rat or mouse) comprises in its genome (e.g., its germline genome) a HoH locus and a ULCiK locus. In some embodiments, a genetically modified rodent (e.g., rat or mouse) is homozygous at a HoH locus, a ULCiK locus, or a combination thereof.

In some embodiments, a genetically modified rodent (e.g., rat or mouse) comprises in its genome (e.g., its germline genome) a HoH locus and a DLCiK locus. In some embodiments, a genetically modified rodent (e.g., rat or mouse) is homozygous at a HoH locus, a DLCiK locus, or a combination thereof.

In some embodiments, a genetically modified rodent (e.g., rat or mouse) comprises in its genome (e.g., its germline genome) a HoH locus and a HULC locus. In some embodiments, a genetically modified rodent (e.g., rat or mouse) is homozygous at a HoH locus, a HULC locus, or a combination thereof.

In some embodiments, a genetically modified rodent (e.g., rat or mouse) comprises in its genome (e.g., its germline genome) a UHC locus and a KoK locus. In some embodiments, a genetically modified rodent (e.g., rat or mouse) comprises in its genome (e.g., its germline genome) a UHC locus and a LoL locus. In some embodiments, a genetically modified rodent (e.g., rat or mouse) comprises in its genome (e.g., its germline genome) a UHC locus, a KoK locus, and a LoL locus. In some embodiments, a genetically modified rodent (e.g., rat or mouse) is homozygous at a UHC locus, a KoK locus, a LoL locus, or a combination thereof.

In some embodiments, a genetically modified rodent (e.g., rat or mouse) comprises in its genome (e.g., its germline genome) a UHC locus, a KoK locus, and a LoK locus. In some embodiments, a genetically modified rodent (e.g., rat or mouse) comprises in its genome (e.g., its germline genome) a UHC locus, a KoK locus, and a LiK locus.

In some embodiments, a genetically modified rodent (e.g., rat or mouse) comprises in its genome (e.g., its germline genome) a UHC locus and a LoK locus. In some embodiments, a genetically modified rodent (e.g., rat or mouse) is homozygous at a UHC locus, a LoK locus, or a combination thereof.

In some embodiments, a genetically modified rodent (e.g., rat or mouse) comprises in its genome (e.g., its germline genome) a UHC locus and a LiK locus. In some embodiments, a genetically modified rodent (e.g., rat or mouse) is homozygous at a UHC locus, a LiK locus, or a combination thereof.

In some embodiments, a genetically modified rodent (e.g., rat or mouse) comprises in its genome (e.g., its germline genome) a UHC locus and a ULC locus. In some embodiments, a genetically modified rodent (e.g., rat or mouse) is homozygous at a UHC locus, a ULC locus, or a combination thereof.

In some embodiments, a genetically modified rodent (e.g., rat or mouse) comprises in its genome (e.g., its germline genome) a UHC locus and a DLC locus. In some embodiments, a genetically modified rodent (e.g., rat or mouse) is homozygous at a UHC locus, a DLC locus, or a combination thereof.

In some embodiments, a genetically modified rodent (e.g., rat or mouse) comprises in its genome (e.g., its germline genome) a UHC locus and a κULC locus. In some embodiments, a genetically modified rodent (e.g., rat or mouse) is homozygous at a UHC locus, a κULC locus, or a combination thereof.

In some embodiments, a genetically modified rodent (e.g., rat or mouse) comprises in its genome (e.g., its germline genome) a UHC locus and a κDLC locus. In some embodiments, a genetically modified rodent (e.g., rat or mouse) is homozygous at a UHC locus, a κDLC locus, or a combination thereof.

In some embodiments, a genetically modified rodent (e.g., rat or mouse) comprises in its genome (e.g., its germline genome) a UHC locus and a ULCiK locus. In some embodiments, a genetically modified rodent (e.g., rat or mouse) is homozygous at a UHC locus, a ULCiK locus, or a combination thereof.

In some embodiments, a genetically modified rodent (e.g., rat or mouse) comprises in its genome (e.g., its germline genome) a UHC locus and a DLCiK locus. In some embodiments, a genetically modified rodent (e.g., rat or mouse) is homozygous at a UHC locus, a DLCiK locus, or a combination thereof.

In some embodiments, a genetically modified rodent (e.g., rat or mouse) comprises in its genome (e.g., its germline genome) a UHC locus and a HULC locus. In some embodiments, a genetically modified rodent (e.g., rat or mouse) is homozygous at a UHC locus, a HULC locus, or a combination thereof.

In some embodiments, a genetically modified rodent (e.g., rat or mouse) comprises in its genome (e.g., its germline genome) a LoH locus and a KoK locus. In some embodiments, a genetically modified rodent (e.g., rat or mouse) comprises in its genome (e.g., its germline genome) a LoH locus and a LoL locus. In some embodiments, a genetically modified rodent (e.g., rat or mouse) comprises in its genome (e.g., its germline genome) a LoH locus, a KoK locus, and a LoL locus. In some embodiments, a genetically modified rodent (e.g., rat or mouse) is homozygous at a LoH locus, a KoK locus, a LoL locus, or a combination thereof.

In some embodiments, a genetically modified rodent (e.g., rat or mouse) comprises in its genome (e.g., its germline genome) a LoH locus, a KoK locus, and a LoK locus. In some embodiments, a genetically modified rodent (e.g., rat or mouse) comprises in its genome (e.g., its germline genome) a LoH locus, a KoK locus, and a LiK locus.

In some embodiments, a genetically modified rodent (e.g., rat or mouse) comprises in its genome (e.g., its germline genome) a LoH locus and a LoK locus. In some embodiments, a genetically modified rodent (e.g., rat or mouse) is homozygous at a LoH locus, a LoK locus, or a combination thereof.

In some embodiments, a genetically modified rodent (e.g., rat or mouse) comprises in its genome (e.g., its germline genome) a LoH locus and a LiK locus. In some embodiments, a genetically modified rodent (e.g., rat or mouse) is homozygous at a LoH locus, a LiK locus, or a combination thereof.

In some embodiments, a genetically modified rodent (e.g., rat or mouse) comprises in its genome (e.g., its germline genome) a LoH locus and a κULC locus. In some embodiments, a genetically modified rodent (e.g., rat or mouse) is homozygous at a LoH locus, a κULC locus, or a combination thereof.

In some embodiments, a genetically modified rodent (e.g., rat or mouse) comprises in its genome (e.g., its germline genome) a LoH locus and a κDLC locus. In some embodiments, a genetically modified rodent (e.g., rat or mouse) is homozygous at a LoH locus, a κDLC locus, or a combination thereof.

In some embodiments, a genetically modified rodent (e.g., rat or mouse) comprises in its genome (e.g., its germline genome) a LoH locus and a ULCiK locus. In some embodiments, a genetically modified rodent (e.g., rat or mouse) is homozygous at a LoH locus, a ULCiK locus, or a combination thereof.

In some embodiments, a genetically modified rodent (e.g., rat or mouse) comprises in its genome (e.g., its germline genome) a LoH locus and a DLCiK locus. In some embodiments, a genetically modified rodent (e.g., rat or mouse) is homozygous at a LoH locus, a DLCiK locus, or a combination thereof.

In some embodiments, a genetically modified rodent (e.g., rat or mouse) comprises in its genome (e.g., its germline genome) a LoH locus and a HULC locus. In some embodiments, a genetically modified rodent (e.g., rat or mouse) is homozygous at a LoH locus, a HULC locus, or a combination thereof.

### Exemplary Rodent Comprising Kappa Universal Light Chain Locus

In some exemplary embodiments of the present invention, genetically modified non-human animals, e.g., rodents, e.g., mice, comprising a genome with one of the immunoglobulin loci restricted in its ability to generate a wide repertoire of variable regions, can be conveniently utilized in the method that depends on repertoire sequence- and mass spectrometry-based analyses of the nonrestricted immunoglobulin chain. In some embodiments, the restricted immunoglobulin chain is a light chain, e.g, a kappa light chain. In some embodiments, a genetically modified rodent comprises in its genome (e.g., its germline genome) an engineered immunoglobulin heavy chain locus (e.g., an engineered endogenous rodent immunoglobulin heavy chain locus) comprising one or more unrearranged human V_{H} gene segments, one or more unrearranged human D_{H} gene segments, and one or more unrearranged human J_{H} gene segments that are upstream of (e.g., operably linked to) one or more rodent (e.g., rat or mouse) immunoglobulin heavy chain constant region genes (e.g., one or more endogenous rodent (e.g., rat or mouse) immunoglobulin heavy chain constant region genes) (i.e., a HoH locus), and an engineered immunoglobulin κ light chain locus (e.g., an engineered endogenous rodent immunoglobulin κ light chain locus) comprising a single rearranged human κ light chain variable region (Vκ/Jκ) upstream of (e.g., operably linked to) a Cκ gene (a κULC locus). Exemplary rodents including a HoH locus and a κULC locus are exemplified in, e.g., U.S. Pat. Nos. 10,130,081 and 10,143,186, each of which is incorporated by reference in its entirety. In some embodiments, a genetically modified rodent (e.g., rat or mouse) is homozygous at a HoH locus and/or a κULC locus. In some embodiments, a genetically modified rodent (e.g., rat or mouse) is homozygous at a HoH locus and a κULC locus.

In some embodiments, one or more unrearranged human V_{H} gene segments at a HoH locus includes at least six human V_{H} gene segments. In some embodiments, one or more unrearranged human V_{H} gene segments at a HoH locus includes at least 18 human V_{H} gene segments. In some embodiments, one or more unrearranged human V_{H} gene segments at a HoH locus includes at least 39 human V_{H} gene segments. In some embodiments, one or more unrearranged human V_{H} gene segments at a HoH locus includes at least 80 human V_{H} gene segments. In some embodiments, one or more unrearranged human D_{H} gene segments at a HoH locus includes at least 27 human D_{H} gene segments. In some embodiments, one or more unrearranged human J_{H} gene segments at a HoH locus includes at least six human J_{H} gene segments.

In some embodiments, one or more unrearranged human V_{H} gene segments at a HoH locus includes at least 18 human V_{H} gene segments, one or more unrearranged human D_{H} gene segments at a HoH locus includes 27 human D_{H} gene segments, and one or more unrearranged human J_{H} gene segments at a HoH locus includes six human J_{H} gene segments. As discussed herein, such an engineered immunoglobulin heavy chain locus is referred to as a “VelocImmune® 1 HoH locus.” In some embodiments, one or more unrearranged human V_{H} gene segments at a HoH locus includes at least 39 human V_{H} gene segments, one or more unrearranged human D_{H} gene segments at a HoH locus includes 27 human D_{H} gene segments, and one or more unrearranged human J_{H} gene segments at a HoH locus includes six human J_{H} gene segments. As discussed herein, such an engineered immunoglobulin heavy chain locus is referred to as a “VelocImmune® 2 HoH locus.” In some embodiments, one or more unrearranged human V_{H} gene segments at a HoH locus includes at least 80 human V_{H} gene segments, one or more unrearranged human D_{H} gene segments at a HoH locus includes 27 human D_{H} gene segments, and one or more unrearranged human J_{H} gene segments at a HoH locus includes six human J_{H} gene segments. As discussed herein, such an engineered immunoglobulin heavy chain locus is referred to as a “VelocImmune® 3 HoH locus.”

In some embodiments, a genetically modified rodent (e.g., rat or mouse) comprising a HoH locus and a κULC locus also includes a genome (e.g., a germline genome) that lacks a functional endogenous rodent Adam6 gene. In some embodiments, a genetically modified rodent (e.g., rat or mouse) comprising a HoH locus and a κULC locus also includes in its genome (e.g., a germline genome) one or more nucleotide sequences encoding one or more rodent ADAM6 polypeptides, functional orthologs, functional homologs, or functional fragments thereof. In some embodiments, one or more rodent ADAM6 polypeptides is or comprises mouse ADAM6a. In some embodiments, one or more rodent ADAM6 polypeptides is or comprises mouse ADAM6b. In some embodiments, one or more rodent ADAM6 polypeptides is or comprises mouse ADAM6a and mouse ADAM6b. Rodents comprising a HoH locus and a κULC locus and including one or more nucleotide sequences encoding one or more rodent ADAM6 polypeptides, functional orthologs, functional homologs, or functional fragments thereof are exemplified in, e.g., U.S. Pat. Nos. 10,130,081, which is incorporated by reference in its entirety. In some embodiments, a genetically modified rodent (e.g., rat or mouse) as provided expresses one or more rodent (e.g., rat or mouse) ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof that are included on the same chromosome as a HoH locus. In some embodiments, a genetically modified rodent (e.g., rat or mouse) as provided has a genome (e.g., a germline genome) comprising a HoH locus comprising one or more nucleotide sequences encoding one or more rodent ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof in place of a human Adam6 pseudogene. In some embodiments, a genetically modified rodent (e.g., rat or mouse) as provided has a genome (e.g., germline genome) comprising one or more nucleotide sequences encoding one or more rodent (e.g., rat or mouse) ADAM6 polypeptides, functional orthologs, functional homologs, or functional fragments thereof that replace a human Adam6 pseudogene.

In some embodiments, a genetically modified rodent comprising a HoH locus and a κULC locus includes a genome (e.g., a germline genome) comprising one or more human V_{H} gene segments comprising a first and a second human V_{H} gene segment, and one or more nucleotide sequences encoding one or more rodent (e.g., rat or mouse) ADAM6 polypeptides, functional orthologs, functional homologs, or functional fragments thereof between the first human V_{H} gene segment and the second human V_{H} gene segment. In some embodiments, a first human V_{H} gene segment is V_{H}1-2 and a second human V_{H} gene segment is V_{H}6-1. In some embodiments, one or more nucleotide sequences encoding one or more rodent (e.g., a rat or mouse) ADAM6 polypeptides, functional orthologs, functional homologs, or functional fragments thereof are between a human V_{H} gene segment and a human D_{H} gene segment.

In some embodiments, one or more nucleotide sequences encoding one or more rodent ADAM6 polypeptides restore or enhance fertility in a male rodent.

In some embodiments, a single rearranged human κ light chain variable region at a κULC locus comprises a human Vκ gene segment and a human Jκ gene segment. In some embodiments, a human Vκ gene segment is a human Vκ1-39 gene segment or a human Vκ 3-20 gene segment. In some embodiments, a human Jκ gene segment is a human Jκ1 gene segment, a human Jκ2 gene segment, a human Jκ3 gene segment, a human Jκ4 gene segment, or a human Jκ5 gene segment. In some embodiments, a human Vκ gene segment is a human Vκ1-39 gene segment, and a human Jκ gene segment is a human Jκ5 gene segment. In some embodiments, a single rearranged human κ light chain variable region at a κULC locus is a human Vκ1-39/10. In some embodiments, a human Vκ gene segment is a human Vκ 3-20 gene segment, and a human Jκ gene segment is a human Jκ1 gene segment. In some embodiments, a single rearranged human κ light chain variable region at a κULC locus is a human Vκ 3-20/Jκ1.

In some embodiments, a κULC locus comprises a non-native leader sequence. In some embodiments, a leader sequence comprises a signal peptide. In some embodiments, a leader sequence comprises a non-native signal peptide.

In some embodiments, a Cκ gene of a κULC locus is a rodent (e.g., rat or mouse) Cκ gene. In some embodiments, a genetically modified rodent (e.g., rat or mouse) is homozygous at a κULC locus. In some embodiments, a genetically modified rodent (e.g., rat or mouse) is heterozygous at a κULC locus.

In some embodiments, a genetically modified rodent (e.g., rat or mouse), which comprises a κULC locus, lacks endogenous Vκ and/or Jκ gene segments that are capable of rearranging to form an endogenous κ light chain variable region. In some embodiments, a genetically modified rodent (e.g., rat or mouse), which comprises a κULC locus, lacks endogenous Vλ and/or Jλ gene segments that are capable of rearranging to form an endogenous λ light chain variable region.

In some embodiments, a genetically modified rodent (e.g., rat or mouse), which comprises a HoH locus and a κULC locus, produces antibodies comprising, inter alia, (a) heavy chains, where each heavy chain comprises a human heavy chain variable domain operably linked to a rodent (e.g., rat or mouse) heavy chain constant domain, and (b) κ light chains, where each κ light chain comprises a human κ light chain variable domain operably linked to a κ light chain constant domain, e.g., in response to antigenic stimulation. In some embodiments, all κ light chains expressed by a genetically modified rodent (e.g., rat or mouse) comprise human κ light chain variable domains expressed from the single rearranged human κ light chain variable region or a somatically hypermutated version thereof.

In some embodiments, a genetically modified rodent (e.g., rat or mouse), which comprises a κULC locus comprising a single human rearranged κ variable region, further comprises a substitution of at least one non-histidine residue in its light chain variable region, e.g., its CDR3 region, with a histidine region. Such genetically modified rodents are described in U.S. Pat. No. 9,801,362, incorporated herein by reference in its entirety. Immunizing genetically modified rodents comprising substitution of non-histidine residues with histidine residues or insertion of histidine residues facilitates identification of antibodies that exhibit pH-dependent properties towards their antigens, using the combination of repertoire sequencing and MS methods described herein and in the Examples.

In some embodiments, the present disclosure provides methods of identifying a human immunoglobulin heavy chain variable domain or CDR sequence (e.g., CDR3 sequence) of an antibody specific for an antigen from a rodent comprising in its germline genome a κULC locus, the method comprising: (i) obtaining a plurality of peptide sequences of human immunoglobulin heavy chain variable domains that were obtained from a sample comprising a population of antibodies produced by a genetically modified rodent immunized with the antigen, and (ii) interrogating a library of human immunoglobulin heavy chain variable domain sequences with the plurality of peptide sequences, wherein the library comprises a plurality of human immunoglobulin heavy chain variable domain sequences encoded by B cells of the immunized rodent.

In some embodiments, the present disclosure provides methods of identifying a human immunoglobulin heavy chain variable domain or CDR sequence (e.g., CDR3 sequence) of an antibody specific for an antigen from a rodent comprising in its germline genome a κULC locus, the method comprising: (i) obtaining a library of human immunoglobulin heavy chain variable domain sequences comprising a plurality of human immunoglobulin heavy chain variable domain sequences encoded by B cells of a rodent immunized with the antigen, and (ii) interrogating the library with a plurality of peptide sequences of human immunoglobulin heavy chain variable domains that were obtained from a sample comprising a population of antibodies produced by the rodent immunized with the antigen.

### Exemplary Rodent Comprising Lambda Universal Light Chain Locus

In other embodiments of the present invention, the method utilizes a restricted lambda light chain. In some embodiments, a genetically modified rodent comprises in its genome (e.g., its germline genome) an engineered immunoglobulin heavy chain locus (e.g., an engineered endogenous rodent immunoglobulin heavy chain locus) comprising one or more unrearranged human V_{H} gene segments, one or more unrearranged human D_{H} gene segments, and one or more unrearranged human J_{H} gene segments that are upstream of (e.g., operably linked to) one or more rodent (e.g., rat or mouse) immunoglobulin heavy chain constant region genes (e.g., one or more endogenous rodent (e.g., rat or mouse) immunoglobulin heavy chain constant region genes) (i.e., a HoH locus), and an engineered immunoglobulin κ light chain locus (e.g., an engineered endogenous rodent immunoglobulin κ light chain locus) comprising a limited human λ light chain variable region repertoire, wherein the limited human λ light chain variable region repertoire comprises a single rearranged human immunoglobulin λ light chain variable region (WM) and is upstream of (e.g., operably linked to) a light chain constant region gene (a ULCiK locus). Rodents including a HoH locus and a ULCiK locus are exemplified in, e.g., WO 2020/247623, which is incorporated by reference in its entirety. In some embodiments, a genetically modified rodent (e.g., rat or mouse) is homozygous at a HoH locus and/or a ULCiK locus. In some embodiments, a genetically modified rodent (e.g., rat or mouse) is homozygous at a HoH locus and a ULCiK locus.

In some embodiments, one or more unrearranged human V_{H} gene segments at a HoH locus includes at least six human V_{H} gene segments. In some embodiments, one or more unrearranged human V_{H} gene segments at a HoH locus includes at least 18 human V_{H} gene segments. In some embodiments, one or more unrearranged human V_{H} gene segments at a HoH locus includes at least 39 human V_{H} gene segments. In some embodiments, one or more unrearranged human V_{H} gene segments at a HoH locus includes at least 80 human V_{H} gene segments. In some embodiments, one or more unrearranged human D_{H} gene segments at a HoH locus includes at least 27 human D_{H} gene segments. In some embodiments, one or more unrearranged human J_{H} gene segments at a HoH locus includes at least six human J_{H} gene segments.

In some embodiments, one or more unrearranged human V_{H} gene segments at a HoH locus includes at least 18 human V_{H} gene segments, one or more unrearranged human D_{H} gene segments at a HoH locus includes 27 human D_{H} gene segments, and one or more unrearranged human J_{H} gene segments at a HoH locus includes six human J_{H} gene segments. As discussed herein, such an engineered immunoglobulin heavy chain locus is referred to as a “VelocImmune® 1 HoH locus.” In some embodiments, one or more unrearranged human V_{H} gene segments at a HoH locus includes at least 39 human V_{H} gene segments, one or more unrearranged human D_{H} gene segments at a HoH locus includes 27 human D_{H} gene segments, and one or more unrearranged human J_{H} gene segments at a HoH locus includes six human J_{H} gene segments. As discussed herein, such an engineered immunoglobulin heavy chain locus is referred to as a “VelocImmune® 2 HoH locus.” In some embodiments, one or more unrearranged human V_{H} gene segments at a HoH locus includes at least 80 human V_{H} gene segments, one or more unrearranged human D_{H} gene segments at a HoH locus includes 27 human D_{H} gene segments, and one or more unrearranged human J_{H} gene segments at a HoH locus includes six human J_{H} gene segments. As discussed herein, such an engineered immunoglobulin heavy chain locus is referred to as a “VelocImmune® 3 HoH locus.”

In some embodiments, a genetically modified rodent (e.g., rat or mouse) comprising a HoH locus and a ULCiK locus also includes a genome (e.g., a germline genome) that lacks a functional endogenous rodent Adam6 gene. In some embodiments, a genetically modified rodent (e.g., rat or mouse) comprising a HoH locus and a ULCiK locus also includes in its genome (e.g., a germline genome) one or more nucleotide sequences encoding one or more rodent ADAM6 polypeptides, functional orthologs, functional homologs, or functional fragments thereof. In some embodiments, one or more rodent ADAM6 polypeptides is or comprises mouse ADAM6a. In some embodiments, one or more rodent ADAM6 polypeptides is or comprises mouse ADAM6b. In some embodiments, one or more rodent ADAM6 polypeptides is or comprises mouse ADAM6a and mouse ADAM6b. Rodents comprising a HoH locus and a ULCiK locus and including one or more nucleotide sequences encoding one or more rodent ADAM6 polypeptides, functional orthologs, functional homologs, or functional fragments thereof are exemplified in, e.g., U.S. Pat. Nos. 10,130,081, which is incorporated by reference in its entirety. In some embodiments, a genetically modified rodent (e.g., rat or mouse) as provided expresses one or more rodent (e.g., rat or mouse) ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof that are included on the same chromosome as a HoH locus. In some embodiments, a genetically modified rodent (e.g., rat or mouse) as provided has a genome (e.g., a germline genome) comprising a HoH locus comprising one or more nucleotide sequences encoding one or more rodent ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof in place of a human Adam6 pseudogene. In some embodiments, a genetically modified rodent (e.g., rat or mouse) as provided has a genome (e.g., germline genome) comprising one or more nucleotide sequences encoding one or more rodent (e.g., rat or mouse) ADAM6 polypeptides, functional orthologs, functional homologs, or functional fragments thereof that replace a human Adam6 pseudogene.

In some embodiments, a genetically modified rodent comprising a HoH locus and a ULCiK locus includes a genome (e.g., a germline genome) comprising one or more human V_{H} gene segments comprising a first and a second human V_{H} gene segment, and one or more nucleotide sequences encoding one or more rodent (e.g., rat or mouse) ADAM6 polypeptides, functional orthologs, functional homologs, or functional fragments thereof between the first human V_{H} gene segment and the second human V_{H} gene segment. In some embodiments, a first human V_{H} gene segment is V_{H}1-2 and a second human V_{H} gene segment is V_{H}6-1. In some embodiments, one or more nucleotide sequences encoding one or more rodent (e.g., a rat or mouse) ADAM6 polypeptides, functional orthologs, functional homologs, or functional fragments thereof are between a human V_{H} gene segment and a human D_{H} gene segment.

In some embodiments, one or more nucleotide sequences encoding one or more rodent ADAM6 polypeptides restore or enhance fertility in a male rodent.

In some embodiments, a single rearranged human λ light chain variable region at a ULC locus comprises a human Vλ gene segment and a human Jλ gene segment. In some embodiments, a human Vλ gene segment is selected from a group consisting of: Vλ4-69, Vλ8-61, Vλ4-60, Vλ6-57, Vλ10-54, Vλ5-52, Vλ1-51, Vλ9-49, Vλ1-47, Vλ7-46, Vλ5-45, Vλ1- 44, Vλ7-43, Vλ1-40, Vλ5-37, Vλ1-36, Vλ3-27, Vλ3-25, Vλ2-23, Vλ3-22, Vλ3-21, Vλ3-19, Vλ2-18, Vλ3-16, Vλ2-14, Vλ3-12, Vλ2-11, Vλ3-10, Vλ3-9, Vλ2-8, Vλ4-3, and Vλ3-1. In some embodiments, a human Vλ gene segment is selected from a group consisting of: Vλ5-52, Vλ1-51, Vλ9-49, Vλ1-47, Vλ7-46, Vλ5-45, Vλ1-44, Vλ7-43, Vλ1-40, Vλ5-37, Vλ1-36, Vλ3-27, Vλ3-25, Vλ2-23, Vλ3-22, Vλ3-21, Vλ3-19, Vλ2-18, Vλ3-16, Vλ2-14, Vλ3-12, Vλ2-11, Vλ3-10, Vλ3-9, Vλ2-8, Vλ4-3, and Vλ3-1. In some embodiments, a human Vλ gene segment is selected from a group consisting of: Vλ1-51, Vλ5-45, Vλ1-44, Vλ1-40, Vλ3-21, and Vλ2-14. In some embodiments, a human Vλ gene segment is Vλ1-51 or Vλ2-14. In some embodiments, a human gene segment is selected from a group consisting of: Jλ1, Jλ2, Jλ3, Jλ6, and Jλ7. In some embodiments, a human gene segment is selected from a group consisting of: Jλ1, Jλ2, Jλ3, and Jλ7. In some embodiments, a human gene segment is Jλ2.

In some embodiments, a ULC locus comprises a non-native leader sequence. In some embodiments, a ULC locus comprises a single rearranged human λ light chain variable region and a Vκ leader sequence. In some embodiments, a leader sequence comprises a signal peptide. In some embodiments, a leader sequence comprises a non-native signal peptide.

In some embodiments, a genetically modified rodent comprises a limited human λ light chain variable region repertoire operably linked to a rodent (e.g., rat or mouse) Cκ or Cλ gene segment (e.g., a mouse Cλ 1 gene segment).

In some embodiments, a human Vλ gene segment is Vλ1-51, a human gene segment is Jλ2, and a light chain constant region gene is a rodent Cλ. (e.g., a mouse Cλ 1). In some embodiments, a human Vλ gene segment is Vλ1-51, a human gene segment is Jλ2, and a light chain constant region gene is a rodent Cκ. In some embodiments, a human Vλ gene segment is Vλ2-14, a human gene segment is Jλ2, and a light chain constant region gene is a rodent Cλ. (e.g., a mouse Cλ 1). In some embodiments, a human Vλ gene segment is Vλ2-14, a human gene segment is Jλ2, and a light chain constant region gene is a rodent Cκ.

In some embodiments, a genetically modified rodent (e.g., rat or mouse) is homozygous at a ULCiK locus. In some embodiments, a genetically modified rodent (e.g., rat or mouse) is heterozygous at a ULCiK locus.

In some embodiments, a genetically modified rodent (e.g., rat or mouse), which comprises a ULCiK locus, lacks endogenous Vκ and/or Jκ gene segments that are capable of rearranging to form an endogenous κ light chain variable region. In some embodiments, a genetically modified rodent (e.g., rat or mouse), which comprises a ULCiK locus, lacks endogenous Vλ and/or gene segments that are capable of rearranging to form an endogenous λ light chain variable region.

In some embodiments, a genetically modified rodent (e.g., rat or mouse), which comprises a HoH locus and a ULC locus, produces antibodies comprising, inter alia, (a) heavy chains, where each heavy chain comprises a human heavy chain variable domain operably linked to a rodent (e.g., rat or mouse) heavy chain constant domain, and (b) light chains, wherein each light chain comprises a human λ light chain variable domain operably linked to a (e.g., rat or mouse) light chain constant domain (e.g., a Cλ or Cκ domain), e.g., in response to antigenic stimulation. In some embodiments, all light chains expressed by B cells of a genetically modified rodent (e.g., rat or mouse), which comprises a ULCiK locus, comprise human λ light chain variable domains expressed from the single rearranged human λ light chain variable region or a somatically hypermutated version thereof.

In some embodiments, the present disclosure provides methods of identifying a human immunoglobulin heavy chain variable domain or CDR sequence (e.g., CDR3 sequence) of an antibody specific for an antigen from a rodent comprising in its germline genome a ULCiK locus, the method comprising: (i) obtaining a plurality of peptide sequences of human immunoglobulin heavy chain variable domains that were obtained from a sample comprising a population of antibodies produced by a genetically modified rodent immunized with the antigen, and (ii) interrogating a library of human immunoglobulin heavy chain variable domain sequences with the plurality of peptide sequences, wherein the library comprises a plurality of human immunoglobulin heavy chain variable domain sequences encoded by B cells of the immunized rodent.

In some embodiments, the present disclosure provides methods of identifying a human immunoglobulin heavy chain variable domain or CDR sequence (e.g., CDR3 sequence) of an antibody specific for an antigen from a rodent comprising in its germline genome a ULCiK locus, the method comprising: (i) obtaining a library of human immunoglobulin heavy chain variable domain sequences comprising a plurality of human immunoglobulin heavy chain variable domain sequences encoded by B cells of a rodent immunized with the antigen, and (ii) interrogating the library with a plurality of peptide sequences of human immunoglobulin heavy chain variable domains that were obtained from a sample comprising a population of antibodies produced by the rodent immunized with the antigen.

### Exemplary Rodent Comprising Universal Heavy Chain Locus

In other embodiments, the restricted immunoglobulin chain in the mouse utilized in the method described herein is a heavy chain. In some embodiments, a genetically modified rodent comprises in its genome (e.g., its germline genome) an engineered immunoglobulin heavy chain locus (e.g., an engineered endogenous rodent immunoglobulin heavy chain locus) comprising a single rearranged human heavy chain variable region upstream of (e.g., operably linked to) one or more rodent (e.g., rat or mouse) constant region genes (i.e., a UHC locus or a common heavy chain locus), and an engineered immunoglobulin κ light chain locus (e.g., an engineered endogenous rodent immunoglobulin κ light chain locus) comprising one or more unrearranged human Vκ gene segments and one or more unrearranged human Jκ gene segments that are upstream of (e.g., operably linked to) a Cκ gene (i.e., a KoK locus). In some embodiments, a genetically modified rodent (e.g., rat or mouse) is homozygous at a UHC locus and/or a KoK locus. In some embodiments, a genetically modified rodent (e.g., rat or mouse) is homozygous at a UHC locus and a KoK locus.

In some embodiments, a single rearranged human heavy chain variable region at a UHC locus comprises a single human V_{H} gene segment, a single human D_{H} gene segment, and a single human J_{H} gene segment. In some embodiments, a single human V_{H} gene segment is a human V_{H}3-23, a single human D_{H} gene segment is a human D_{H}4-4, and a single human J_{H} gene segment is a human J_{H}4.

In some embodiments, a single rearranged human heavy chain variable region at a UHC locus comprises a single human V_{H} gene segment and a single human J_{H} gene segment, which are separated by two amino acids. In some embodiments, a single human V_{H} gene segment is a human V_{H}3-23, a single human J_{H} gene segment is a human J_{H}4, and two amino acids are glycine and tyrosine.

In some embodiments, one or more rodent (e.g., mouse or rat) heavy chain constant region genes at a UHC locus are one or more endogenous rodent (e.g., mouse or rat) heavy chain constant region genes.

In some embodiments, a genetically modified rodent (e.g., rat or mouse) comprising a UHC locus and a KoK locus lacks a functional endogenous rodent Adam6 gene. In some embodiments, a genetically modified rodent (e.g., rat or mouse) comprising a UHC locus and a KoK locus includes one or more nucleotide sequences encoding one or more rodent ADAM6 polypeptides, functional orthologs, functional homologs, or functional fragments thereof. In some embodiments, one or more rodent ADAM6 polypeptides is or comprises mouse ADAM6a. In some embodiments, one or more rodent ADAM6 polypeptides is or comprises mouse ADAM6b. In some embodiments, one or more rodent ADAM6 polypeptides is or comprises mouse ADAM6a and mouse ADAM6b. In some embodiments, a genetically modified rodent (e.g., rat or mouse) as provided expresses one or more rodent (e.g., rat or mouse) ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof that are included on the same chromosome as a UHC locus. In some embodiments, a genetically modified rodent (e.g., rat or mouse) as provided has a genome (e.g., a germline genome) comprising a UHC locus comprising one or more nucleotide sequences encoding one or more rodent ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof in place of a human Adam6 pseudogene. In some embodiments, a genetically modified rodent (e.g., rat or mouse) as provided has a genome (e.g., germline genome) comprising one or more nucleotide sequences encoding one or more rodent (e.g., rat or mouse) ADAM6 polypeptides, functional orthologs, functional homologs, or functional fragments thereof that replace a human Adam6 pseudogene.

In some embodiments, one or more nucleotide sequences encoding one or more rodent ADAM6 polypeptides restore or enhance fertility in a male rodent.

In some embodiments, one or more unrearranged human Vκ gene segments at a KoK locus includes at least six human Vκ gene segments. In some embodiments, one or more unrearranged human Vκ gene segments at a KoK locus includes at least 16 human Vκ gene segments. In some embodiments, one or more unrearranged human Vκ gene segments at a KoK locus includes at least 30 human Vκ gene segments. In some embodiments, one or more unrearranged human Vκ gene segments at a KoK locus includes at least 40 human Vκ gene segments. In some embodiments, one or more unrearranged human Jκ gene segments at a KoK locus includes at least five human Jκ gene segments.

In some embodiments, one or more unrearranged human Vκ gene segments at a KoK locus includes at least 16 human Vκ gene segments, and one or more unrearranged human Jκ gene segments includes at least five human Jκ gene segments. As described herein, such an engineered immunoglobulin heavy chain locus is referred to herein as a “VelocImmune® 1 KoK locus.” In some embodiments, one or more unrearranged human Vκ gene segments at a KoK locus includes at least 30 human Vκ gene segments, and one or more unrearranged human Jκ gene segments at a KoK locus includes at least five human Jκ gene segments. As described herein, such an engineered immunoglobulin heavy chain locus is referred to herein as a “VelocImmune® 2 KoK locus.” In some embodiments, one or more unrearranged human Vκ gene segments at a KoK locus includes at least 40 human Vκ gene segments, and one or more unrearranged human Jκ gene segments at a KoK locus includes at least five human Jκ gene segments. As described herein, such an engineered immunoglobulin heavy chain locus is referred to herein as a “VelocImmune® 3 KoK locus.”

In some embodiments, an immunoglobulin κ light chain constant region gene of a KoK locus is a rodent (e.g., rat or mouse) CK gene. In some embodiments, an immunoglobulin light chain constant region gene of a KoK locus is an endogenous rodent (e.g., rat or mouse) CK gene. In some embodiments, an immunoglobulin κ light chain constant region gene of a KoK locus is an endogenous rodent (e.g., rat or mouse) CK gene at an endogenous immunoglobulin light chain locus. In some embodiments, a genetically modified rodent (e.g., rat or mouse) is homozygous at a KoK locus. In some embodiments, a genetically modified rodent (e.g., rat or mouse) is heterozygous at a KoK locus.

In some embodiments, a genetically modified rodent (e.g., rat or mouse), which comprises a UHC locus and a KoK locus, produces antibodies comprising, inter alia, (a) heavy chains, where each heavy chain comprises a human heavy chain variable domain operably linked to a rodent (e.g., rat or mouse) heavy chain constant domain, and (b) K light chains, where each K light chain comprises a human κ light chain variable domain operably linked to a rodent (e.g., rat or mouse) K light chain constant domain, e.g., in response to antigenic stimulation. In some embodiments, all heavy chains expressed by a genetically modified rodent (e.g., rat or mouse) comprise human heavy chain variable domains expressed from the single rearranged human heavy chain variable region or a somatically hypermutated version thereof.

In some embodiments, a genetically modified rodent (e.g., rat or mouse) comprising a UHC locus and a KoK locus also comprises an exogenous terminal deoxynucleotidyl transferase (TdT) gene. In some embodiments, a rodent (e.g., rat or mouse) that comprises an exogenous terminal deoxynucleotidyl transferase (TdT) gene can have increased antigen receptor diversity when compared to a rodent without an exogenous TdT gene.

In some embodiments, a rodent as described herein has a genome comprising an exogenous terminal deoxynucleotidyltransferase (TdT) gene operably linked to a transcriptional control element.

In some embodiments, a transcriptional control element includes a RAG1 transcriptional control element, a RAG2 transcriptional control element, an immunoglobulin heavy chain transcriptional control element, an immunoglobulin κ light chain transcriptional control element, an immunoglobulin λ light chain transcriptional control element, or any combination thereof.

In some embodiments, an exogenous TdT is located at an immunoglobulin κ light chain locus, an immunoglobulin λ light chain locus, an immunoglobulin heavy chain locus, a RAG1 locus, or a RAG2 locus.

In some embodiments, a TdT is a human TdT. In some embodiments, a TdT is a short isoform of TdT (TdTS).

In some embodiments, the present disclosure provides methods of identifying a human immunoglobulin light chain variable domain or CDR sequence (e.g., CDR3 sequence) of an antibody specific for an antigen from a rodent comprising in its germline genome a UHC locus, the method comprising: (i) obtaining a plurality of peptide sequences of human immunoglobulin light chain variable domains that were obtained from a sample comprising a population of antibodies produced by a genetically modified rodent immunized with the antigen, and (ii) interrogating a library of human immunoglobulin light chain variable domain sequences with the plurality of peptide sequences, wherein the library comprises a plurality of human immunoglobulin light chain variable domain sequences encoded by B cells of the immunized rodent.

In some embodiments, the present disclosure provides methods of identifying a human immunoglobulin light chain variable domain or CDR sequence (e.g., CDR3 sequence) of an antibody specific for an antigen from a rodent comprising in its germline genome a UHC locus, the method comprising: (i) obtaining a library of human immunoglobulin light chain variable domain sequences comprising a plurality of human immunoglobulin light chain variable domain sequences encoded by B cells of a rodent immunized with the antigen, and (ii) interrogating the library with a plurality of peptide sequences of human immunoglobulin light chain variable domains that were obtained from a sample comprising a population of antibodies produced by the rodent immunized with the antigen.

### Generated Antigen-Specific Antibodies

After an antibody of interest (e.g., variable domain of interest and/or CDR sequence(s) of interest) has been identified from genetically modified non-human animal (e.g., rodent, e.g., rat or mouse) using a method described herein, the method may further comprise expressing a nucleotide sequence encoding the obtained antibody (i.e., first antibody) or portion thereof (e.g., variable region), in an antigen-binding protein or a second, recombinant antibody. In some embodiments, an antibody sequence identified by the methods described herein is subsequently expressed in a host cell. In some embodiments, a variable region sequence of an antibody identified herein is cloned into a second recombinant antibody that is expressed in a host cell. Various embodiments of second recombinant antibody are described herein below. In various embodiments, the antibody obtained by the method described herein is further tested to confirm binding to the antigen immunogen, or to determine kinetic binding parameters of the antibody. In some embodiments, supernatants or purified proteins from cells expressing (e.g., transfected with) the second antibody obtained by the method described herein, are screened in a variety of assays to determine binding affinity and/or specificity for the antigen. Various assays that can be used include those described in the foregoing examples, and others that will be apparent to those skilled in the art. In various embodiments, the antibody specifically binds to the antigen of interest or to the epitope on the antigen of interest (e.g., with a K_{D} in the micromolar, nanomolar, or picomolar range).

In some embodiments, a nucleotide sequence encoding the obtained antibody is from an immunized host (e.g., genetically modified non-human animal, e.g., a rodent, e.g., a mouse or a rat), that comprises in its genome (e.g., its germline genome) a restricted repertoire of heavy and/or light chain variable regions. In some embodiments, a nucleotide sequence encoding a heavy chain variable domain is obtained from an immunized host (e.g., genetically modified non-human animal, e.g., a rodent, e.g., a mouse or a rat), that comprises in its genome (e.g., its germline genome) a restricted immunoglobulin light chain variable region repertoire. In some embodiments, a nucleotide sequence encoding a light chain variable domain is obtained from an immunized host (e.g., genetically modified non-human animal, e.g., a rodent, e.g., a mouse or a rat), that comprises in its genome (e.g., its germline genome) a restricted immunoglobulin heavy chain variable region repertoire.

In some embodiments, a nucleotide sequence encoding a heavy chain variable domain is obtained from an immunized rodent (e.g. mouse) that comprises in its genome (e.g., its germline genome) a single rearranged human light chain variable region comprising a single light chain V gene segment and a single light chain J gene segment, e.g., a single human light chain Vκ gene segment and a single human light chain Jκ gene segment or a single human light chain Vλ gene segment and a single human light chain Jλ gene segment (rodent comprising a ULC locus, see., e.g., U.S. Pat. Nos. 10,143,186 and 10,130,081, incorporated herein by reference in their entireties). Thus, upon immunization of such rodent (e.g., mouse) with an antigen of interest, the method described herein allows analysis of heavy chain variable region (e.g., heavy chain CDR3) sequences of antibodies directed against the antigen of interest, and selection of a heavy chain variable region sequence.

In some embodiments, a nucleotide sequence encoding the obtained antibody from an immunized host (e.g., genetically modified non-human animal, e.g., a rodent, e.g., a mouse or a rat) is codon optimized. In some embodiments, a nucleotide sequence encoding an obtained heavy chain and/or light chain variable domain is codon optimized. In some embodiments, a nucleotide sequence encoding one or more obtained CDR sequences are codon optimized.

In some embodiments, the obtained nucleotide sequence encoding the human immunoglobulin variable domain (e.g., heavy chain and/or light chain variable region) is inserted into a construct for expression of an antigen-binding protein. In some embodiments, an antigen-binding protein is an antibody.

In some embodiments, the obtained nucleotide sequence encoding the human immunoglobulin variable domain is inserted into a construct in operable linkage with a human immunoglobulin constant region, such that the antibody is expressed as a fully human antibody, with the human variable region upstream of a human constant region. Thus, in some embodiments, the method further comprises, subsequent to obtaining nucleotide sequence encoding a human immunoglobulin heavy chain variable domain and/or a human immunoglobulin light chain variable domain as described herein, (i) joining or ligating the nucleotide sequence encoding the human immunoglobulin heavy chain variable domain to a nucleotide sequence encoding a human immunoglobulin heavy chain constant domain, thereby forming a human immunoglobulin heavy chain sequence encoding a fully human immunoglobulin heavy chain, and/or (ii) joining or ligating the nucleotide sequence encoding the human immunoglobulin light chain variable domain (e.g., human immunoglobulin κ and/or λ light chain variable domain) to a nucleotide sequence encoding a human immunoglobulin light chain constant domain (e.g., human immunoglobulin κ and/or λ light chain constant domain), thereby forming a human immunoglobulin κ and/or λ light chain sequence encoding a fully human immunoglobulin κ and/or λ light chain. In certain embodiments, a human immunoglobulin heavy chain sequence, and a human immunoglobulin κ and/or λ light chain sequence are expressed in a cell (e.g., a host cell, a mammalian cell) so that fully human immunoglobulin heavy chains and fully human immunoglobulin κ and/or λ light chains are expressed and form human antibodies. In some embodiments, human antibodies are isolated from the cell or culture media including the cell.

In some embodiments the antigen-binding protein (e.g., second antibody) is a human antibody and/or a bispecific antibody. The phrase “bispecific antibody” includes an antibody capable of selectively binding two or more epitopes. Bispecific antibodies generally comprise two non-identical heavy chains, with each heavy chain specifically binding a different epitope—either on two different molecules (e.g., different epitopes on two different immunogens) or on the same molecule (e.g., different epitopes on the same immunogen). If a bispecific antibody is capable of selectively binding two different epitopes (a first epitope and a second epitope), the affinity of the first heavy chain for the first epitope will generally be at least one to two or three or four or more orders of magnitude lower than the affinity of the first heavy chain for the second epitope, and vice versa. Epitopes specifically bound by the bispecific antibody can be on the same or a different target (e.g., on the same or a different protein). Bispecific antibodies can be made, for example, by combining heavy chains that recognize different epitopes of the same immunogen. For example, nucleic acid sequences encoding heavy chain variable sequences that recognize different epitopes of the same immunogen can be fused to nucleic acid sequences encoding the same or different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer epitope-binding specificity but that can associate with each heavy chain, or that can associate with each heavy chain and that can bind one or more of the epitopes bound by the heavy chain epitope-binding regions, or that can associate with each heavy chain and enable binding of one or both of the heavy chains to one or both epitopes.

For example, where the antigen-binding protein (e.g., second antibody) is a bispecific antibody, in some embodiments, the bispecific antibody is generated by immunizing a genetically modified non-human animal, e.g., a rodent, e.g., a mouse or a rat, that comprises in its genome (e.g., its germline genome) a restricted repertoire of heavy and/or light chain variable regions. In some embodiments, the non-human animal is a mouse and the mouse comprises in its genome (e.g., its germline genome) a single rearranged human light chain variable region comprising a single light chain V gene segment and a single light chain J gene segment, e.g., a single human light chain Vκ gene segment and a single human light chain Jκ gene segment or a single human light chain Vλ gene segment and a single human light chain Jλ gene segment (rodent comprising a ULC locus, see., e.g., U.S. Pat. Nos. 10,143,186 and 10,130,081, incorporated herein by reference in their entireties). Thus, upon immunization of such mouse with a first antigen of interest, the method described herein allows analysis of heavy chain variable region (e.g., heavy chain CDR3) sequences of antibodies directed against the first antigen of interest, and selection of a first heavy chain variable region sequence for use in a bispecific antibody. The method is repeated in order to obtain a second heavy chain variable region against a second antigen of interest, by immunizing a second mouse also comprising a single rearranged human light chain variable region comprising a single light chain V gene segment and a single light chain J gene segment (e.g., the same light chain V and J gene segments as present in the first mouse), and obtaining the second heavy chain variable region from said second mouse using the method described herein. Alternatively, the second heavy chain variable region sequence can be obtained using the methods known in the art (e.g., hybridoma technology or other methods described in U.S. Pat. Nos. 10,143,186 and 10,130,081, incorporated herein by reference in their entireties). The first and the second heavy chain variable regions are expressed in a first and second heavy chain (e.g., first and second human heavy chain) together with the same light chain as present in the first and second mouse, or a somatically mutated version thereof, to generate a bispecific antibody.

In some embodiments, e.g., where the antigen-binding protein (e.g., second antibody) is a bispecific antibody, the obtained nucleotide sequence encoding the human immunoglobulin variable domain, e.g., human immunoglobulin heavy chain variable domain, is inserted into a construct in operable linkage with a human heavy chain immunoglobulin constant region, wherein the Fc domain of the heavy chain comprises modifications to facilitate heavy chain heterodimer formation and/or to inhibit heavy chain homodimer formation. Such modifications are provided, for example, in U.S. Pat. Nos. 5,731,168, 5,807,706, 5,821,333, 7,642,228 and 8,679,785 and in U.S. Pat. Pub. No. 2013/0195849, each of which is hereby incorporated by reference. In yet another embodiment, e.g., where the second antibody is a bispecific antibody, the obtained nucleotide sequence encoding the human immunoglobulin variable domain, e.g., human immunoglobulin heavy chain variable domain, is inserted into a construct in operable linkage with a human heavy chain immunoglobulin constant region (e.g., human IgG constant region) wherein one of the heavy chains of the bispecific antibody is modified to omit a Protein A-binding determinant, resulting in a differential affinity of a homodimeric antigen binding protein from a heterodimeric antigen binding protein. As such, one immunoglobulin heavy chain of the bispecific antibody comprises a first CH3 region of a human IgG selected from IgG1, IgG2, and IgG4, wherein the first CH3 region binds to Protein A, and a second immunoglobulin heavy chain comprises a second CH3 region of a human IgG selected from IgG1, IgG2, and IgG4, wherein the second CH3 region comprises a modification that reduces or eliminates binding of the second CH3 region to Protein A, while an immunoglobulin light chain of the bispecific antibody pairs with both immunoglobulin heavy chains. Compositions and methods that address this issue are described in U.S. Pat. No. 9,309,326, hereby incorporated by reference in its entirety.

In some embodiments, the nucleotide sequence encoding the human variable domain obtained by the methods described herein is expressed in a cell line in operable linkage with a human immunoglobulin constant region, such that a fully human antibody is generated. In some embodiments, the cell line that expresses the fully human antibody is any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF −9, SF −21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vera, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HTl 080 cell, 10 myeloma cell, tumor cell, and a cell line derived from an aforementioned cell. In some embodiments, the cell comprises one or more viral genes, e.g., a retinal cell that expresses a viral gene (e.g., a PER.C6™ cell).

Mammalian host cells used to produce the antibody may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. Media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as, e.g., gentamycin), trace elements (defined as inorganic compounds usually present at final concentrations in the micromolar range), and glucose or an equivalent energy source. Any other supplements may also be included at appropriate concentrations as known to those skilled in the art. The culture conditions, such as temperature, pH, and the like, are, in various embodiments, those previously used with the host cell selected for expression, and will be apparent to those skilled in the art.

### Methods of Making Antigen Binding Proteins and Nucleic Acid Sequences Encoding the Same

The disclosure herein describes a method for obtaining an amino acid and/or nucleotide sequence of a light chain and/or heavy chain of an antibody from a host (i.e. genetically modified host described herein) immunized with an antigen of interest.

In some embodiments, a method comprises obtaining a nucleotide sequence encoding a human immunoglobulin variable domain of a first antibody specific for said antigen, comprising: obtaining from a first sample from the immunized host comprising a plurality of nucleic acid sequences that encode a plurality of immunoglobulin variable domains and determining amino acid sequences of the plurality of immunoglobulin variable domains, obtaining from the immunized host a second sample comprising a population of antibodies directed against the antigen of interest and determining therefrom peptide sequences of heavy and/or light chain variable domains of the population of antibodies, interrogating the amino acids sequences of the plurality of immunoglobulin variable domains with the peptide sequences of heavy and/or light chain variable domains of the population of antibodies, thereby obtaining a sequence of a human variable domain of an antibody specific for the antigen, and obtaining a nucleotide sequence encoding a human immunoglobulin variable domain of the antibody specific for the antigen. In some embodiments, the method further comprises utilizing the obtained nucleotide sequence encoding a human immunoglobulin variable domain in an antigen-binding protein (e.g., a second antibody). In some embodiments, a nucleotide sequence encoding a human immunoglobulin variable domain in an antigen-binding protein is codon optimized.

In some embodiments, provided herein is a method of obtaining a nucleotide sequence encoding a human immunoglobulin variable domain of an antibody specific for an antigen, comprising: obtaining from a first sample from a host immunized with the antigen a plurality of nucleic acid sequences that encode a plurality of immunoglobulin variable domains and determining amino acid sequences of the encoded plurality of immunoglobulin variable domains; obtaining from the immunized host a second sample comprising a population of antibodies directed against the antigen of interest and determining therefrom peptide sequences of heavy and/or light chain variable domains of the population of antibodies; interrogating the amino acids sequences of the encoded plurality of immunoglobulin variable domains with the peptide sequences heavy and/or light chain variable domains of the population of antibodies, thereby obtaining a human immunoglobulin variable domain of an antibody specific for the antigen; and obtaining a nucleotide sequence encoding the human immunoglobulin variable domain of the antibody specific for the antigen.

In some embodiments, provided herein is a method of obtaining a nucleotide sequence encoding a human immunoglobulin variable domain CDR (e.g., CDR3) sequence of an antibody specific for an antigen, comprising: obtaining from a first sample from a host immunized with the antigen a plurality of nucleic acid sequences that encode a plurality of immunoglobulin variable domains and determining amino acid sequences of the encoded plurality of immunoglobulin variable domains; obtaining from the immunized host a second sample comprising a population of antibodies directed against the antigen of interest and determining therefrom peptide sequences of heavy and/or light chain variable domains of the population of antibodies; interrogating the amino acids sequences of the plurality of human immunoglobulin variable domains with the peptide sequences of heavy and/or light chain variable domains of the population of antibodies from the second sample, thereby obtaining a human immunoglobulin variable domain CDR, (e.g., CDR3), sequence of an antibody specific for the antigen, and obtaining a nucleotide sequence encoding the human immunoglobulin variable domain CDR, (e.g., CDR3), sequence of the antibody specific for the antigen.

In some embodiments, provided herein is a method of obtaining a human immunoglobulin variable domain sequence of an antibody specific for an antigen, comprising: obtaining from a first sample from a host immunized with the antigen a plurality of nucleic acid sequences that encode a plurality of immunoglobulin variable domains and determining amino acid sequences of the encoded plurality of immunoglobulin variable domains; obtaining from the immunized host a second sample comprising a population of antibodies directed against the antigen of interest and determining therefrom peptide sequences of heavy and/or light chain variable domains of the population of antibodies; interrogating the amino acids sequences of the plurality of immunoglobulin variable domains, thereby obtaining a human immunoglobulin variable domain sequence of an antibody specific for the antigen.

In some embodiments, provided herein is a method of obtaining a human immunoglobulin variable domain CDR (e.g., CDR3) sequence of an antibody specific for an antigen, comprising: obtaining from a first sample from a host immunized with the antigen a plurality of nucleic acid sequences that encode a plurality of immunoglobulin variable domains and determining amino acid sequences of the encoded plurality of immunoglobulin variable domains, obtaining from the immunized host a second sample comprising a population of antibodies directed against the antigen of interest and determining therefrom peptide sequences of heavy and/or light chain variable domains of the population of antibodies, interrogating the amino acids sequences of the plurality of immunoglobulin variable domains with the peptide sequences of heavy and/or light chain variable domains of the population of antibodies, thereby obtaining a human immunoglobulin variable domain CDR, e.g., CDR3, sequence of an antibody specific for the antigen.

Thus, in some embodiments, provided herein is a nucleic acid sequence encoding human immunoglobulin variable domain or encoding human immunoglobulin variable domain CDR (e.g, CDR3) obtained using the methods described herein. In other embodiments, provided herein is a nucleic acid sequence encoding an immunoglobulin light or heavy chain obtained using the methods described herein.

In some embodiments, also provided herein is an amino acid sequence of human variable domain or CDR (e.g., CDR3) obtained using the methods described herein. In other embodiments, provided herein is an amino acid sequence of an immunoglobulin light or heavy chain obtained using the methods described herein.

In some embodiments, also provided herein is a method for making an antibody comprising: expressing in a host cell (i) a nucleic acid encoding an immunoglobulin heavy chain comprising a human immunoglobulin heavy chain variable region sequence operably linked to an immunoglobulin heavy chain constant region sequence and (ii) a nucleic acid encoding an immunoglobulin light chain comprising a human immunoglobulin light chain variable region sequence operably linked to an immunoglobulin light chain constant region sequence, wherein the human immunoglobulin heavy chain variable region sequence and/or the human immunoglobulin light chain variable region sequence were identified by any of the methods provided herein. In some embodiments, the host cell is cultured under conditions such that the host cell expresses an antibody comprising the immunoglobulin heavy chain and the immunoglobulin light chain.

In some embodiments, also provided herein is a method of making a fully human immunoglobulin heavy chain and/or fully human immunoglobulin light chain comprising: (a) identifying a human immunoglobulin heavy chain and/or light chain variable domain sequence by any of the methods provided herein; (b) operably linking the nucleic acid encoding the human immunoglobulin heavy chain variable domain with a nucleic acid encoding a human immunoglobulin heavy chain constant domain to form a fully human immunoglobulin heavy chain and/or operably linking the nucleic acid encoding the human immunoglobulin light chain variable domain with a nucleic acid encoding a human immunoglobulin light chain constant domain to form a fully human immunoglobulin light chain; and (c) expressing the fully human immunoglobulin heavy chain and/or fully human immunoglobulin light chain. In some embodiments, the fully human immunoglobulin heavy chain and/or fully human immunoglobulin light chain are expressed in a host cell.

In some embodiments, also provided herein is an antibody comprising the sequences obtained using the methods described herein.

In some embodiments, provided is a cell expressing the antigen-binding protein derived from the human immunoglobulin variable domain obtained by the methods described herein. In some embodiments, the cell is a cell line used for manufacture of the antigen-binding protein, e.g., manufacture of the antigen-binding protein for administration to a subject.

### Pharmaceutical Compositions

In some embodiments, an antigen-binding protein, a nucleic acid encoding an antigen-binding protein, or a therapeutically relevant portion thereof produced by a method disclosed herein or derived from an antibody, a nucleic acid, or a therapeutically relevant portion thereof produced by a method disclosed herein can be administered to a subject (e.g., a human subject). In some embodiments, a pharmaceutical composition includes an antibody produced by a non-human animal disclosed herein. In some embodiments, a pharmaceutical composition can include a buffer, a diluent, an excipient, or any combination thereof. In some embodiments, a composition, if desired, can also contain one or more additional therapeutically active substances.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with routine, if any, experimentation.

For example, a pharmaceutical composition provided herein may be in a sterile injectable form (e.g., a form that is suitable for subcutaneous injection or intravenous infusion). For example, in some embodiments, a pharmaceutical composition is provided in a liquid dosage form that is suitable for injection. In some embodiments, a pharmaceutical composition is provided as powders (e.g., lyophilized and/or sterilized), optionally under vacuum, which can be reconstituted with an aqueous diluent (e.g., water, buffer, salt solution, etc.) prior to injection. In some embodiments, a pharmaceutical composition is diluted and/or reconstituted in water, sodium chloride solution, sodium acetate solution, benzyl alcohol solution, phosphate buffered saline, etc. In some embodiments, a powder should be mixed gently with the aqueous diluent (e.g., not shaken).

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

In some embodiments, a pharmaceutical composition including an antibody produced by a method disclosed herein can be included in a container for storage or administration, for example, a vial, a syringe (e.g., an IV syringe), or a bag (e.g., an IV bag). A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, a pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, a composition may comprise between 0.1% and 100% (w/w) active ingredient.

A pharmaceutical composition may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of a pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

In some embodiments, a provided pharmaceutical composition comprises one or more pharmaceutically acceptable excipients (e.g., preservative, inert diluent, dispersing agent, surface active agent and/or emulsifier, buffering agent, etc.). In some embodiments, a pharmaceutical composition comprises one or more preservatives. In some embodiments, a pharmaceutical composition comprises no preservative.

In some embodiments, a pharmaceutical composition is provided in a form that can be refrigerated and/or frozen. In some embodiments, a pharmaceutical composition is provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of antibody compositions for longer than the specified time results in antibody degradation.

Liquid dosage forms and/or reconstituted solutions may comprise particulate matter and/or discoloration prior to administration. In some embodiments, a solution should not be used if discolored or cloudy and/or if particulate matter remains after filtration.

General considerations in the formulation and/or manufacture of pharmaceutical agents may be found, for example, in Remington: The Science and Practice of Pharmacy 21st ed., Lippincott Williams & Wilkins, 2005, incorporated herein by reference.

### Kits

The present disclosure further provides a pack or kit comprising one or more containers filled with at least protein (single or complex (e.g., an antibody or fragment thereof)), obtained by method as described herein. Kits may be used in any applicable method (e.g., a research method). Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, and/or (c) a contract that governs the transfer of materials and/or biological products (e.g., a non-human animal or non-human cell as described herein) between two or more entities and combinations thereof.

In some embodiments, a kit comprising an amino acid (e.g., an antibody or fragment thereof) obtained by method as described herein is provided. In some embodiments, a kit comprising a nucleic acid (e.g., a nucleic acid encoding an antibody or fragment thereof) encoding an antibody or an antigen-binding fragment thereof obtained by a method as described herein is provided. In some embodiments, a kit comprising a sequence (amino acid and/or nucleic acid sequence) identified by a method described herein is provided.

In some embodiments, a kit as described herein for use in the manufacture and/or development of a drug (e.g., an antibody or fragment thereof) for therapy or diagnosis is provided.

In some embodiments, a kit as described herein for use in the manufacture and/or development of a drug (e.g., an antibody or fragment thereof) for the treatment, prevention or amelioration of a disease, disorder or condition is provided.

Other features of certain embodiments will become apparent in the course of the following descriptions of exemplary embodiments, which are given for illustration and are not intended to be limiting thereof.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXEMPLARY EMBODIMENTS

Embodiment 1. A method of obtaining from a host immunized with a particular antigen a human immunoglobulin variable domain or a CDR of an antibody specific for said antigen, comprising: (i) obtaining from a first sample from the immunized host a plurality of nucleic acids encoding a plurality of human immunoglobulin variable domains and determining amino acid sequences of the encoded plurality of immunoglobulin variable domains, (ii) obtaining from the immunized host a second sample comprising a population of antibodies directed against the antigen and determining therefrom peptide sequences of heavy and/or light chain variable domains of the population of antibodies, (iii) interrogating the amino acid sequences of the encoded plurality of human immunoglobulin variable domains from the first sample with the peptide sequences of the heavy and/or light chain variable domains of the population of antibodies from the second sample, thereby obtaining a human immunoglobulin variable domain or CDR sequence of an antibody specific for the antigen; wherein the host is a genetically modified non-human mammal that comprises in its genome an immunoglobulin heavy chain variable region comprising one or more human heavy chain V gene segment, one or more human D gene segment, and one or more human heavy chain J gene segment, wherein the heavy chain variable region is operably linked to a constant region, and an immunoglobulin light chain variable region comprising one or more human light chain V gene segment and one or more human light chain J gene segment, wherein the light chain is operably linked to a constant region.

Embodiment 2. The method of embodiment 1, wherein the host is a rodent.

Embodiment 3. The method of embodiment 2, wherein the host is a rat.

Embodiment 4. The method of embodiment 2, wherein the host is a mouse.

Embodiment 5. The method of embodiment 1, wherein the first sample comprises a population of B cells.

Embodiment 6. The method of embodiment 5, wherein the first sample is a bone marrow sample and/or a spleen sample.

Embodiment 7. The method of any one of the preceding embodiments, wherein the obtaining from the first sample a plurality of nucleic acid sequences that encode a plurality of immunoglobulin variable domains comprises preparing cDNA from the nucleic acid sequences and sequencing rearranged heavy chain VDJ sequences and/or rearranged light chain VJ sequences in the first sample.

Embodiment 8. The method of embodiment 7, wherein the obtaining from the first sample a plurality of nucleic acids encoding a plurality immunoglobulin variable domains, is determined using DNA sequencing technology.

Embodiment 9. The method of embodiment 8, wherein the DNA sequencing technology is next generation DNA sequencing.

Embodiment 10. The method of any one of the preceding embodiments, wherein the second sample is selected from the group consisting of serum, plasma, lymphoid organs, gut, cerebrospinal fluid, brain, spinal cord, or placenta.

Embodiment 11. The method of any one of the preceding embodiments, wherein the determining from the second sample peptide sequences comprises mass spectrometric analysis of the heavy and/or light chain variable domains of the population of antibodies in the second sample.

Embodiment 12. The method of embodiment 11, wherein the mass spectrometric analysis combines liquid chromatography and mass spectrometry (LC-MS).

Embodiment 13. The method of embodiment 11 or 12, wherein the method further comprises prior to mass spectrometric analysis a proteolytic digestion of the heavy and/or light chain variable domains of the population of antibodies.

Embodiment 14. The method of any one of the preceding embodiments, wherein obtaining from the immunized host a second sample comprising a population of antibodies directed against the particular antigen comprises depleting the second sample of antibodies not directed against the particular antigen.

Embodiment 15. The method of any one of the preceding embodiments, wherein obtaining from the immunized host a second sample comprising a population of antibodies directed against the particular antigen comprises enriching the second sample for antibodies directed against the particular antigen.

Embodiment 16. The method of any one of the preceding embodiments, wherein interrogating the amino acid sequences of the plurality of immunoglobulin variable domains from the first sample with the peptide sequences of the heavy and/or light chain variable domains of the population of antibodies from the second sample comprises aligning peptide sequences of heavy and/or light chain variable domains of the population of antibodies to each other and to the amino acid sequences of the plurality of immunoglobulin variable domains.

Embodiment 17. The method of any one of the preceding embodiments further comprising obtaining a nucleotide sequence of the human variable domain of the antibody specific for the antigen.

Embodiment 18. The method of embodiment 17, wherein the method further comprises expressing the obtained nucleotide sequence encoding the human immunoglobulin variable domain in a second, recombinant antibody.

Embodiment 19. The method of embodiment 18, wherein the nucleotide sequence encoding the human variable domain is expressed in a cell line in operable linkage with a human immunoglobulin constant region.

Embodiment 20. The method of embodiment 19, wherein the human variable domain is a human heavy chain variable domain expressed in operable linkage with a human immunoglobulin heavy chain constant region to generate a human immunoglobulin heavy chain.

Embodiment 21. The method of embodiment 20, wherein the human immunoglobulin heavy chain is expressed in a cell line with a human immunoglobulin light chain.

Embodiment 22. The method of embodiment 19, wherein the human variable domain is a human light chain variable domain expressed in operable linkage with a human immunoglobulin light chain constant region to generate a human immunoglobulin light chain.

Embodiment 23. The method of embodiment 22, wherein the human immunoglobulin light chain is expressed in a cell line with a human immunoglobulin heavy chain.

Embodiment 24. The method of any one of embodiments 18-23, wherein the second antibody is a fully human antibody.

Embodiment 25. The method of any one of embodiments 18-24, wherein the second antibody is a bispecific antibody.

Embodiment 26. The method of any one of embodiments 18-25, wherein the method further comprises purifying the second antibody and determining affinity and/or specificity of the purified second antibody for a particular antigen.

Embodiment 27. The method of any one of the preceding embodiments, wherein the host is a genetically modified mouse that comprises in its genome an immunoglobulin heavy chain variable region comprising one or more human heavy chain V gene segment, one or more human D gene segment, and one or more human heavy chain J gene segment, wherein the heavy chain variable region is operably linked to a murine constant region, and an immunoglobulin light chain variable region comprising one or more human light chain V gene segment and one or more human light chain J gene segment, wherein the light chain is operably linked to a murine constant region.

Embodiment 28. The method of embodiment 27, wherein the immunoglobulin heavy chain variable region is operably linked to a mouse heavy chain constant region, and/or the immunoglobulin light chain variable region is operably linked to a mouse light chain constant region.

Embodiment 29. The method of embodiment 28, wherein the immunoglobulin heavy chain variable region operably linked to a mouse heavy chain constant region is at the endogenous mouse heavy chain locus, and/or the immunoglobulin light chain variable region operably linked to a mouse light chain constant region is at the endogenous mouse light chain locus.

Embodiment 30. The method of any one of embodiments 27-29, wherein the host is a genetically modified mouse that comprises in its genome, including in its germline genome, an immunoglobulin heavy chain variable region comprising a plurality of human heavy chain V gene segments, a plurality of human D gene segments, and a plurality of human heavy chain J gene segments, wherein the heavy chain variable region is operably linked to a murine heavy chain constant region, and an immunoglobulin light chain variable region comprising exactly two unrearranged human Vκ gene segments and five unrearranged human Jκ gene segments operably linked to a murine light chain constant region, wherein the exactly two unrearranged human Vκ gene segments are a human Vκ1-39 gene segment and a human Vκ 3-20 gene segment.

Embodiment 31. The method of embodiment 27, wherein the host is a genetically modified mouse whose genome comprises (a) at an endogenous heavy chain locus: (i) an immunoglobulin heavy chain variable region comprising a plurality of unrearranged human V_{H} gene segments, a plurality of unrearranged human D_{H} gene segments, and a plurality of unrearranged human J_{H} gene segments operably linked to a mouse heavy chain constant region; (ii) a restricted unrearranged heavy chain variable region, comprising a single human V_{H} gene segment, one or more unrearranged human D_{H} gene segments, and one or more unrearranged human J_{H} gene segments, operably linked to a mouse heavy chain constant region; (iii) a universal heavy chain encoding sequence comprising a single rearranged human heavy chain variable region operably linked to a mouse heavy chain constant region; (iv) a histidine modified unrearranged heavy chain variable region, comprising one or more unrearranged human V_{H} gene segments, one or more unrearranged human D_{H} gene segments, and one or more unrearranged human J_{H} gene segments, further comprising substitution or insertion of at least one histidine for a non-histidine residue, operably linked to a mouse heavy chain constant region; (v) a heavy chain only immunoglobulin encoding sequence comprising an immunoglobulin heavy chain variable region, comprising one or more unrearranged human V_{H} gene segments, one or more unrearranged human D_{H} gene segments, and one or more unrearranged human J_{H} gene segments, operably linked to a heavy chain constant region wherein a non-IgM gene, e.g., an IgG gene, lacks a sequence that encodes a functional CH1 domain; or (vi) an engineered endogenous rodent immunoglobulin heavy chain locus comprising one or more unrearranged human V_{L} gene segments and one or more unrearranged human J_{L} gene segments, operably linked to a mouse immunoglobulin heavy chain constant region gene; and/or (b) at an endogenous light chain locus: (i) an immunoglobulin light chain variable region comprising a plurality of unrearranged human Vκ gene segments and a plurality of unrearranged human Jκ gene segments operably linked to a mouse light chain constant region; (ii) a universal light chain encoding sequence comprising a single rearranged human light chain variable region, operably linked to a mouse light chain constant region; (iii) a restricted light chain variable region, comprising two unrearranged human Vκ gene segments and one or more unrearranged human Jκ gene segments, operably linked to a mouse light chain constant region; or (iv) a histidine modified light chain variable region comprising one or more human light chain V gene segments and one or more human light chain J gene segments, further comprising substitution or insertion of at least one histidine for a non-histidine residue, operably linked to a mouse light chain constant region.

Embodiment 32. The method of any one of the preceding embodiments, wherein the genetically modified mouse further comprises a functional ADAM6 gene, optionally wherein the functional ADAM6 gene is a mouse ADAM6 gene.

Embodiment 33. The method of any one of the preceding embodiments, wherein the genetically modified mouse further expresses an exogenous terminal deoxynucleotidyl transferase (TdT) gene.

Embodiment 34. A method of obtaining from a host immunized with a particular antigen a human immunoglobulin heavy chain variable domain or a CDR of an antibody specific for said antigen, comprising: obtaining from a first sample from the immunized host a plurality of nucleic acids encoding a plurality of human immunoglobulin heavy chain variable domains and determining amino acid sequences of the encoded plurality of human immunoglobulin variable domains, obtaining from the immunized host a second sample comprising a population of antibodies directed against the particular antigen and determining therefrom peptide sequences of human heavy chain variable domains of the population of antibodies, interrogating the amino acids sequences of the plurality of human immunoglobulin heavy chain variable domains with the peptide sequences of the human heavy chain variable domains of the population of antibodies thereby obtaining a human immunoglobulin heavy chain variable domain or a CDR of an antibody specific for the antigen; wherein the host is a genetically modified mouse that comprises in its genome, including in its germline genome; an immunoglobulin heavy chain variable region comprising a plurality of human heavy chain V gene segments, a plurality of human D gene segments, and a plurality of human heavy chain J gene segments, wherein the heavy chain variable region is operably linked to a murine constant region, and an immunoglobulin light chain variable region which is a single rearranged human light chain variable region comprising a single human light chain V gene segment and a single human light chain J gene segment, wherein the human immunoglobulin light chain variable region is operably linked to a murine light chain constant region.

Embodiment 35. The method of embodiment 34, wherein the single rearranged human light chain variable region is a single rearranged human kappa light chain variable region comprising a single human light chain Vκ gene segment and a single human light chain Jκ gene segment.

Embodiment 36. The method of embodiment 35, wherein the single human light chain Vκ gene segment is a Vκ1-39 or Vκ3-20 gene segment, and the single human light chain Jκ gene segment is a Jκ1 or a Jκ5 gene segment.

Embodiment 37. The method of embodiment 35, wherein the murine light chain constant region is a mouse kappa light chain constant region.

Embodiment 38. The method of embodiment 35, wherein the single rearranged human light chain variable region is operably liked to a mouse light chain constant region at the endogenous mouse kappa light chain locus.

Embodiment 39. The method of any one of embodiments 35-38, wherein the genetically modified mouse further comprises a functional ADAM6 gene, optionally wherein the functional ADAM6 gene is a mouse ADAM6 gene.

Embodiment 40. The method of embodiment 39, wherein the first sample comprises a population of B cells.

Embodiment 41. The method of embodiment 40, wherein the first sample is a bone marrow sample and/or a spleen sample.

Embodiment 42. The method of any one of embodiments 34-41, wherein the obtaining from the first sample a plurality of nucleic acid sequences encoding a plurality of human immunoglobulin heavy chain variable domains comprises preparing cDNA from the nucleic acid sequences and sequencing rearranged heavy chain VDJ sequences in the first sample.

Embodiment 43. The method of embodiment 42, wherein the obtaining from the first sample a plurality of nucleic acid sequences that encode a plurality of immunoglobulin variable domains is determined using DNA sequencing technology.

Embodiment 44. The method of embodiment 43, wherein the DNA sequencing technology is next generation DNA sequencing.

Embodiment 45. The method of any one of embodiments 34-44, wherein the second sample is selected from the group consisting of serum, plasma, lymphoid organs, gut, cerebrospinal fluid, brain, spinal cord, or placenta.

Embodiment 46. The method of any one of embodiments 34-45, wherein the determining from the second sample peptide sequences comprises mass spectrometric analysis of the heavy chain variable domains of the population of antibodies in the second sample.

Embodiment 47. The method of embodiment 46, wherein the mass spectrometric analysis combines liquid chromatography and mass spectrometry (LC-MS).

Embodiment 48. The method of embodiment 46 or 47, wherein the method further comprises prior to mass spectrometric analysis a proteolytic digest of the heavy chain variable domains of the population of antibodies.

Embodiment 49. The method of any one of embodiments 34-48, wherein obtaining from the immunized host a second sample comprising a population of antibodies directed against the particular antigen comprises depleting the second sample of antibodies not directed against the particular antigen.

Embodiment 50. The method of any one of embodiments 34-49, wherein obtaining from the immunized host a second sample comprising a population of antibodies directed against the particular antigen comprises enriching the second sample for antibodies directed against the particular antigen.

Embodiment 51. The method of any one of embodiments 34-50, wherein interrogating the amino acid sequences of the plurality of human immunoglobulin heavy chain variable domains with the peptide sequences of human heavy chain variable domains of the population of antibodies comprises aligning the peptide sequences of human heavy chain variable domains of the population of antibodies to each other and to the amino acid sequences of the plurality of human immunoglobulin heavy chain variable domains.

Embodiment 52. The method of any one of embodiments 34-51, further comprising obtaining a nucleotide sequence of the human heavy chain variable domain of the antibody specific for the antigen.

Embodiment 53. The method of embodiment 52, wherein the method further comprises expressing the obtained nucleotide sequence encoding the human immunoglobulin heavy chain variable domain in a second, recombinant antibody.

Embodiment 54. The method of embodiment 53, wherein the nucleotide sequence encoding the human heavy chain variable domain is expressed in a cell line in operable linkage with a human immunoglobulin heavy constant region to generate a human immunoglobulin heavy chain.

Embodiment 55. The method of embodiment 54, wherein the human immunoglobulin heavy chain is expressed in a cell line with a human immunoglobulin light chain.

Embodiment 56. The method of embodiment 55, wherein the human immunoglobulin light chain is derived from the same single rearranged variable region sequence as present in the mouse, or a somatically mutated version thereof.

Embodiment 57. The method of any one of embodiments 53-56, wherein the second antibody is a human antibody.

Embodiment 58. The method of any one of embodiments 53-57, wherein the second antibody is a bispecific antibody.

Embodiment 59. The method of any one of embodiments 53-58, wherein the method further comprises purifying the second antibody and determining affinity and/or specificity of the purified second antibody for the particular antigen.

Embodiment 60. The method of any one of the preceding embodiments, wherein the obtaining a human immunoglobulin heavy chain variable domain or a CDR of an antibody specific for the antigen is based on one or more of: (1) a match of a unique peptide obtained from the second sample to a CDR3 sequence in the amino acid sequence obtained from the first sample; (2) a match of unique peptides obtained from the second sample to CDR1 and/or CDR2 sequences in the amino acid sequence obtained from the first sample, (3) a match of one or more unique peptide obtained from the second sample to one or more framework sequences in the amino acid sequence obtained from the first sample, (4) the number of next generation sequencing counts, (5) exclusion of CDR sequence with methionine, and (6) exclusion of CDR sequence with potential N glycosylation.

Embodiment 61. A method of obtaining an immunoglobulin variable domain or a CDR of an antibody specific for an antigen, comprising: obtaining a sample comprising a population of antibodies directed against an antigen from a host immunized with the antigen, and determining peptide sequences of heavy and/or light chain variable domains of the population of antibodies, interrogating peptide sequences of heavy and/or light chain variable domains of the population of antibodies from the sample with a library of amino acid sequences comprising a plurality of human immunoglobulin variable domains, thereby obtaining a human immunoglobulin variable domain or CDR sequence of an antibody specific for the antigen; wherein the immunized host is a genetically modified non-human mammal that comprises in its germline genome: an immunoglobulin heavy chain variable region comprising one or more human heavy chain V gene segments, one or more human D gene segments, and one or more human heavy chain J gene segments, wherein the heavy chain variable region is operably linked to a constant region, and an immunoglobulin light chain variable region comprising one or more human light chain V gene segments and one or more human light chain J gene segments, wherein the light chain is operably linked to a constant region.

Embodiment 62. The method of embodiment 61, wherein the library of amino acid sequences comprising a plurality of human immunoglobulin variable domains is encoded by a plurality of nucleic acids obtained from the host immunized with the antigen, wherein the immunized host is genetically modified non-human mammal that comprises in its germline genome: an immunoglobulin heavy chain variable region comprising one or more human heavy chain V gene segments, one or more human D gene segments, and one or more human heavy chain J gene segments, wherein the heavy chain variable region is operably linked to a constant region, and an immunoglobulin light chain variable region comprising one or more human light chain V gene segments, and one or more human light chain J gene segments, wherein the light chain is operably linked to a constant region.

Embodiment 63. The method of embodiments 61-62, wherein the sample is selected from the group consisting of serum, plasma, lymphoid organs, gut, cerebrospinal fluid, brain, spinal cord, or placenta.

Embodiment 64. The method of embodiments 62-63, wherein the library of amino acid sequences comprising a plurality of human immunoglobulin variable domains is encoded by a plurality of nucleic acids obtained from a B cells sample which is a bone marrow and/or a spleen sample.

Embodiment 65. A method for identifying a human immunoglobulin variable domain or CDR of an antibody specific for a particular antigen, the method comprising comparing a plurality of amino acid sequences encoded by a plurality of nucleic acids that encode a plurality of human immunoglobulin variable domains produced by an animal immunized with said antigen with amino acid sequences comprising peptide fragments from light chain and/or heavy chain variable domains produced from a population of antibodies directed against the antigen; and thereby identifying a human immunoglobulin variable domain or CDR of an antibody specific for said antigen, wherein said animal is a genetically modified non-human mammal that comprises in its genome an immunoglobulin heavy chain variable region comprising one or more human heavy chain V gene segment, one or more human D gene segment, and one or more human heavy chain J gene segment, wherein the heavy chain variable region is operably linked to a constant region, and an immunoglobulin light chain variable region comprising one or more human light chain V gene segment and one or more human light chain J gene segment, wherein the light chain is operably linked to a constant region.

Embodiment 66. The method of embodiment 65, wherein the plurality of nucleic acids and peptide fragments are obtained from the animal immunized with the antigen.

### EXAMPLES

The invention is further illustrated by the following non-limiting examples. These Examples are set forth to aid in the understanding of the invention but are not intended to, and should not be construed to, limit its scope in any way. The Examples do not include detailed descriptions of conventional methods that would be well-known to those of ordinary skill in the art (molecular cloning techniques, etc.). Unless indicated otherwise, parts are parts by weight, molecular weight is average molecular weight, and temperature is indicated in Celsius. One having ordinary skill in the art would understand that the order of steps are not necessarily absolute and can vary to achieve the same outcome in certain embodiments.

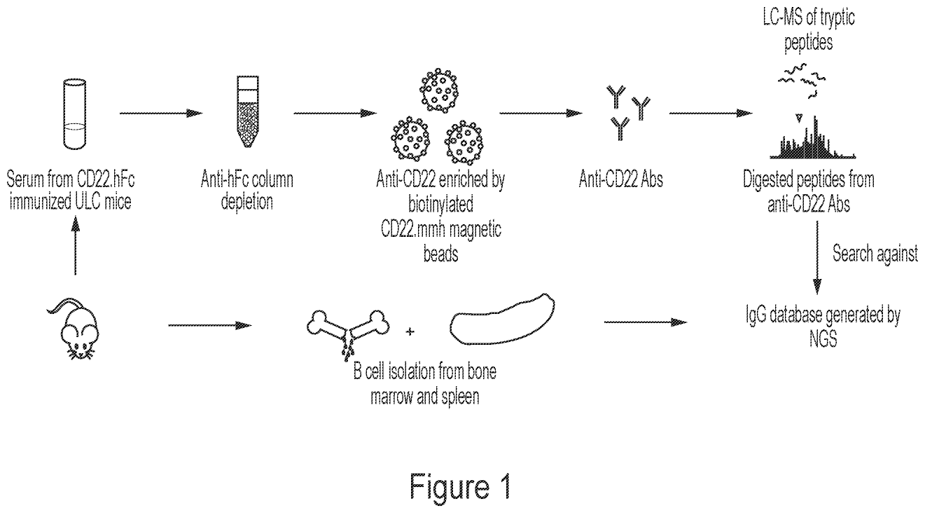

An exemplary overview of the process is provided herein in FIG. 1. Briefly, and as described in the following examples, a rodent (e.g., a mouse or rat) is immunized with an antigen of interest (such as, e.g., CD22-Fc fusion protein), and anti-antigen titers are assessed. An animal whose bleeds exhibit high anti-antigen titers is sacrificed, bone marrow and/or spleen are obtained, and B cells purified and processed by Next Generation Sequencing (NGS) to generate a database of immunoglobulin sequences (e.g., variable domain sequences, e.g., heavy chain variable domain sequences). Serum (or an alternative desired sample) is also obtained from the same sacrificed animal, and is enriched for antigen-specific antibodies (in an exemplary embodiment below, depleted for anti-Fc titers and enriched for anti-CD22 titers); antigen-enriched antibodies are enzymatically digested into peptides and these peptides are sequenced by mass spectrometry. Digested peptide sequences are searched against the generated NGS database to determine the variable domain sequences (e.g., heavy chain variable domain sequences) of antibodies specific against the antigen of interest.

### Example 1. Immunization of Universal Light Chain Mice

Immunization

Kappa Universal Light Chain (κULC) Mice (mice comprising either a single rearranged human Vk1-39Jκ5 or Vk3-20Jκ1, operably linked to a mouse Cκ, and also comprising a plurality of human heavy chain V, D, and J gene segments operably linked to a mouse heavy chain constant region; mice referred to as ULC1-39 or ULC3-20, respectively) were immunized with human CD22.Fc chimera (hCD22.hFc) immunogen. Kappa universal light chain mice were previously described, e.g., in U.S. Pat. Nos. 10,130,081, 10,143,186 and US 2019/0090462, which are incorporated in their entirely herein. Pre-immune serum was collected from the mice prior to the initiation of immunization. The mice were boosted at varying time intervals using standard adjuvants and immunization protocols. The mice were bled periodically, and anti-serum titers were assayed on respective antigens.

Anti-Serum Titer Determination

On Protein:

Antibody titers in serum against immunogen were determined on protein using ELISA. Ninety-six (96)-well microtiter plates (Thermo Scientific) were coated with 2 μg/ml each of hCD22 or human Fc proteins in phosphate-buffered saline (PBS, Irvine Scientific) overnight at 4° C. Plates were washed with phosphate-buffered saline containing 0.05% Tween 20 (PBS-T, Sigma-Aldrich) and blocked with 300 μl of 0.5% bovine serum albumin (BSA, Sigma-Aldrich) in PBS for 1 h at room temperature. Pre-immune and immune anti-sera were serially diluted three-fold in 0.5% BSA-PBS and added to the plates for 1 h at room temperature. The plates were washed and goat anti-mouse IgG-Fc-Horse Radish Peroxidase (HRP) conjugated secondary antibody (Jackson Immunoresearch) was added to the plates and incubated for 1 h at room temperature. Plates were washed and developed using TMB/H₂O₂ as substrate according to manufacturer's recommended procedure and absorbance at 450 nm were recorded using a spectrophotometer (Vκ tor, Perkin Elmer). Antibody titers were computed using Graphpad PRISM software, with antibody titer defined as interpolated serum dilution factor of which the binding signal is 2-fold over background.

On Cells:

Antibody titers in serum against immunogen were determined on cells using Meso Scale Discovery (MSD) cell binding ELISA. Ninety-six (96)-well carbon surface plates were coated with 40,000 cells/well of Raji and Jurkat cells in PBS at 37° C. for 1 hour. The cell coating solution was decanted and the plates were blocked with 150 μL of 2% bovine serum albumin (BSA, Sigma-Aldrich) in PBS for 1 h at room temperature (RT). Plates were washed with PBS three times using a plate washer (AquaMax®2000 from Molecular Devices). Pre-immune and immune anti-sera were serially diluted three-fold in 1% BSA-PBS and added to the plates for 1 h at room temperature. The plates were washed and goat anti-mouse IgG-Fc ruthenium conjugated secondary antibody was then added to the plates at 1 μg/mL and incubated for 1 hour at RT. Plates were washed and developed by adding 150 μl per well MSD's 4× surfactant free Read Buffer T (diluted to 1×) and read on MSD SECTOR™ imager 600 instrument. Anti-serum titers were computed using Graphpad PRISM software, with antibody titer defined as interpolated serum dilution factor of which the binding signal is 2-fold over background.

Results

The humoral immune responses in ULC1-39 and ULC3-20 mice were investigated following immunization with hCD22 protein immunogen. Antibody titers in serum were determined on human CD22 and human Fc proteins using ELISA and on Raji and Jurkat cells using MSD cell binding assays. Antisera from the mice showed high titers to hCD22 and hFc proteins. High specific titers were elicited on Raji cells (Table 1). The antibody titer was defined as interpolated serum dilution factor of which the binding signal is 2-fold over background.

TABLE 1
Antibody Titers form CD22 Fc Immunized Mice

2nd bleed titers

| | CD22 Fc | hFc | Raji | Jurkat |
|---|---|---|---|---|
| Strain | protein | protein | cells | cells |
| ULC 1-39 | 777,930 | 270,684 | 376,452 | 7,384 |
| mouse 1 | | | | |
| ULC 1-39 | 539,202 | 307,925 | 199,552 | 6,256 |
| mouse 2 | | | | |
| ULC 3-20 | 985,618 | 523,236 | 286,168 | 7,800 |

Spleens and bone marrow from all mice were harvested for next generation sequencing (NGS) experiments. Serum from each mouse was used in Liquid Chromatography Mass Spectrometry (LC-MS) experiments.

### Example 2. Next Generation Sequencing and Construction of a Reference Antibody Database

### Example 2.1. Next Generation Sequencing (NGS)

Next Generation Sequencing, or Repertoire sequencing, was performed on mouse bone marrow and splenocytes. Bone marrow was collected from the femurs of CD22 immunized mice by flushing the femurs with 1× phosphate buffered saline (PBS, Gibco) containing 2.5% fetal bovine serum (FBS). Single cell suspensions were prepared from mouse spleens. Red blood cells from spleen and bone marrow preparation were lysed with ACK lysis buffer (Gibco). Splenic B cells were positively enriched from total splenocytes by magnetic cell sorting using anti-CD19 (mouse, a marker for B cells) magnetic beads and MACS® columns (Miltenyi Biotech). Each mouse tissue was processed in four replicates for repertoire sequencing. Total RNA was isolated from bone marrow and purified splenic B cells using an RNeasy Plus RNA isolation kit (Qiagen) according to manufacturer's instructions.

Reverse transcription was performed to generate human heavy chain cDNA containing IgG constant region sequence, using a SMARTer™ RACE cDNA Amplification Kit (Clontech) and an oligo-dT primer. During reverse transcription, a DNA sequence, which is a reverse complement of the template switching (TS) primer, was attached to the 3′ end of newly synthesized cDNAs. Purified cDNAs were amplified by two rounds of semi-nested PCR to generate a plurality of cDNAs encoding the total IgG variable domain complement expressed by cell from which mRNA was obtained, followed by a third round of PCR to attach sequencing primers and indexes. Exemplary primers used for IgG repertoire library construction are provided in Table 2.

TABLE 2
Primers used in library preparation for IgG Repertoire Sequencing

| Template switching (TS) | | 5′ -CACCATCGATGTCGACACGCCTArGrGrG -3′ |
|---|---|---|
| primer | | (SEQ ID NO. 1) |
| RT primer | Oligo-dT | |
| 1^{st} round PCR | IgG constant | A mixture (1:1:1:1) of the following 4 primers: |
| primers | | 5′ -GGAAGGTGTGCACACCGCTGGAC -3′ (SEQ ID NO. 2) |
| | | 5' -GGAAGGTGTGCACACTGCTGGAC -3′ (SEQ ID NO. 3) |
| | | 5′ -GGAAGGTGTGCACACCACTGGAC -3′ (SEQ ID NO. 4) |
| | | 5′ -AGACTGTGCGCACACCGCTGGAC -3′ (SEQ ID NO. 5) |
| | TS specific | 5′ -AAGCAGTGGTATCAACGCAGAGTACAT -3′ (SEQ ID NO. 6) |
| 2^{nd} round | IgG constant | A mixture (1:1:1:1) of the following 4 primers: |
| PCR primers | | 5′ -ACACTCTTTCCCTACACGACGCTCTTCCGATCT |
| | | AGTGGATAGACAGATGGGGGTG -3′ (SEQ ID NO. 7) |
| | | 5′ -ACACTCTTTCCCTACACGACGCTCTTCCGATCT |
| | | AGTGGATAGACTGATGGGGGTG -3′ (SEQ ID NO. 8) |
| | | 5′ -ACACTCTTTCCCTACACGACGCTCTTCCGATCT |
| | | AGTGGATAGACCGATGGGGCTG-3′ (SEQ ID NO. 9) |
| | | 5′ -ACACTCTTTCCCTACACGACGCTCTTCCGATCT |
| | | AAGGGATAGACAGATGGGGCTG -3′ (SEQ ID NO. 10) |
| | TS specific | 5′ - GTGACTGGAGTTCAGACGTGTGCTCTTCCGATCT |
| | | CACCATCGATGTCGACACGCCTA- 3′ (SEQ ID NO. 11) |
| Final round | Forward | 5′ - |
| PCR Primers | | AATGATACGGCGACCACCGAGATCTACACXXXXXX |
| | | ACACTCTTTCCCTACACGACGCTCTTCCGATCT- 3′ |
| | | (SEQ ID NO. 12) |
| | Reverse | 5′ - CAAGCAGAAGACGGCATACGAGATXXXXXX |
| | | GTGACTGGAGTTCAGACGTGTGCTCTTCCGATCT- 3′ |
| | | (SEQ ID NO. 13) |

“XXXXXX” represents a 6 base pair index sequence to enable multiplexing samples for sequencing

Human variable domain cDNAs were size selected tor 400-700 bp using Pippin Prep (SAGE Science) and quantified by qPCR using a KAPA Library Quantification Kit (KAPA Biosystems) before loading samples onto a Miseq sequencer (Illumina) for sequencing for 2×300 cycles.

### Example 2.2. Antibody Reference Database Construction

Mouse-specific protein sequence databases were constructed using variable diversity joining (VDJ) region sequences from ULC mice, grouping by tissue for each mouse sample. VDJ sequence data obtained from NGS was first de-muliplexed and filtered based on quality, length and perfect match to IgG constant region primer. Overlapping paired-end reads were merged and analyzed using a local installation of publicly available IgBLAST (NCBI, v2.2.25+) to align rearranged heavy chain sequences to human germline V and J gene database. CDR3 sequences were extracted using International Immunogenetics Information System (IMGT) boundaries. IMGT clonotype (AA) was defined as a unique V-(D)-J rearrangement, with conserved CDR3-IMGT anchors (cysteine C 104, tryptophan W 118 or phenylalanine F 118), and a unique CDR3-IMGT AA junction sequence. Frequency of occurrence of each protein sequence and HCDR3 was calculated. For reference sequence database construction used for antibody identification via MS, single read sequences were excluded to reduce impact of sequencing errors.

Additional filters were applied to remove nonproductive sequences with stop codons and out-of-frame re-arrangements. Truncated sequences containing incomplete alignment of framework regions were also removed during creation of the database.

In total, 6,452,901 reads were obtained from bone marrow and spleen samples.

VDJ encoding sequences were collapsed based on amino acid sequence and a total of 927,191 unique full-length in-frame VDJ genes were used in construction of the reference sequence database. Results from all tissues of CD22-immunized ULC mice were used to construct the database, which can be interrogated by the variable domain peptides identified from the serum-derived antibodies.

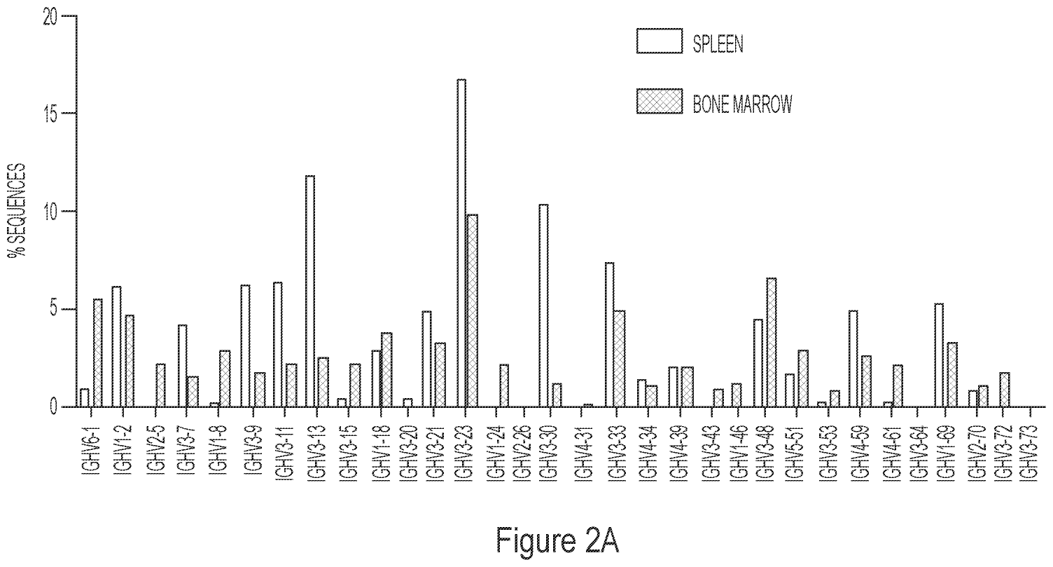

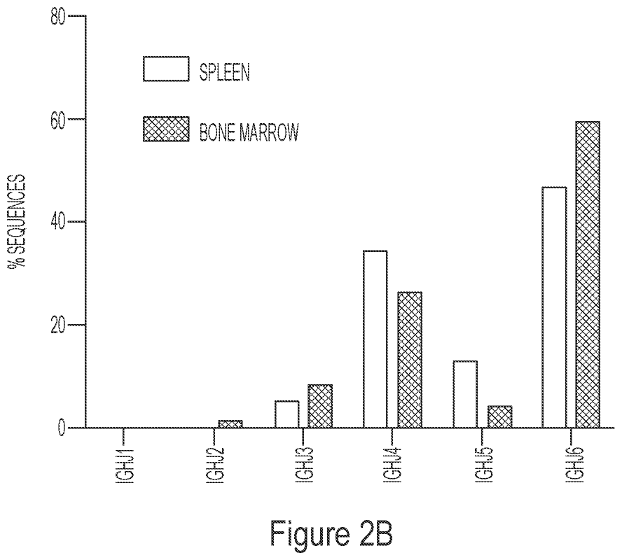

Gene usage and antibody clonotypes comprising serum IgG repertoire were delineated in ULC mice immunized with CD22. Diverse heavy chain variable gene segments (IGHV; FIG. 2A) and heavy chain joining gene segments (IGHJ: FIG. 2B) usage were identified in spleen and bone marrow. Number of distinct HCDR3 sequences detected in spleen and bone marrow samples are summarized in Table 3. The number of distinct human CDR3 sequences increased with the increased number of reads (data not shown).

TABLE 3
Number of antibody and HCDR3 sequences detected

| | | BM | | SPLEEN | |
|---|---|---|---|---|---|
| | | VDJ SEQ | HCDR3 | VDJ SEQ | HCDR3 |
| Genotype | Mouse | (AA) | (AA) | (AA) | (AA) |
| 1-39ULC | MOUSE 1 | 84063 | 15735 | 100959 | 19031 |
| | MOUSE 2 | 110724 | 16382 | 118602 | 21482 |
| 3-20ULC | MOUSE 3 | 116469 | 14295 | 147600 | 19299 |
| | MOUSE 4 | 153940 | 27015 | 94834 | 17718 |

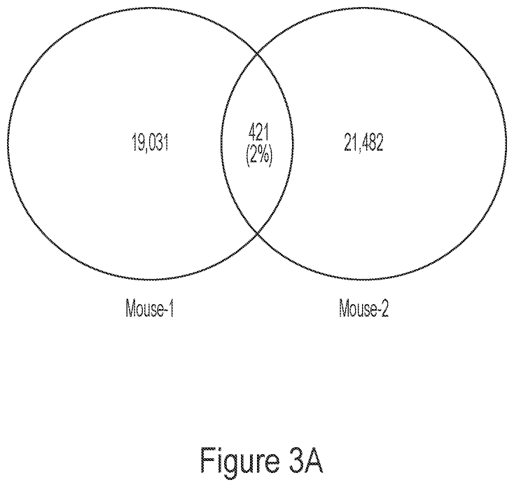

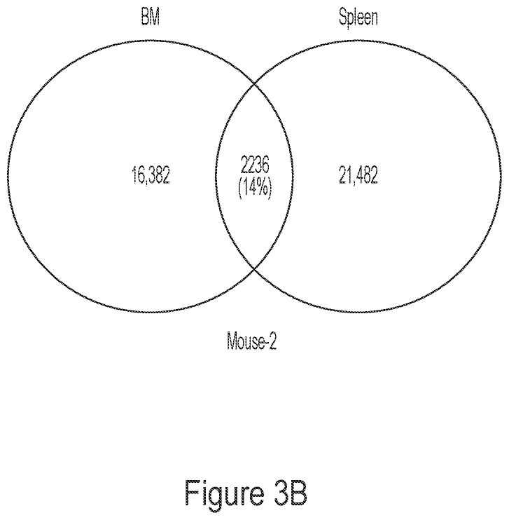

A limited number of public HCDR3 amino acid sequences were observed across different mice. HCDR3 sequences observed in more than one mouse in the same tissue comprised 2%. (FIG. 3A). 10-14% of HCDR3 amino acid sequences were found to be shared between bone marrow and spleen samples of the same mouse (FIG. 3B). The mouse-specific reference sequence database generated by high-throughput sequencing pipeline was used to interpret peptide mass spectra obtained through the proteomics analysis (see FIG. 1).

### Example 3. Exemplary Enrichment of Antibodies with Desired Characteristics by Affinity Capture of Anti-hFc and Anti-hCD22

### Example 3.1. Anti-hFc Depletion of Serum

Serum from all ULC mice contained antibody titers against hCD22 and hFc. Sequential affinity capture steps were applied to isolate anti-hFc antibodies and anti-hCD22 antibodies, respectively (FIG. 1). Immunized ULC mouse serum samples were PBS-diluted to a final volume of 1 mL and passed through a hFc conjugated agarose column to deplete anti-Fc antibodies from the sample. PBS (1 mL) was added to the column, flow-throughs combined and concentrated to a final volume of 100 μL using a 300 dalton (molecular weight) cut-off filter. Anti-hFc depleted serum flow-throughs were used downstream for anti-CD22 enrichment. The agarose column was washed 3× with 1 mL of 20 mM Tris-HCl, pH8.0 and once with 1 mL of ddH2O. Bound anti-hFc antibodies were eluted with 2 mL of 300 mM acetic acid. The anti-hFc antibody eluant was Speedvac dried and proteins separated via SDS-gel and proteins prepared for LC-MS analysis (subsequent data for anti-Fc antibodies is not shown).

### Example 3.2. Anti-CD22 Antibody Isolation

Anti-CD22 antibodies were isolated from anti-hFc depleted serum sample. Biotinylated human CD22 extracellular domain polypeptide (100 μg/mL) was immobilized onto streptavidin paramagnetic beads (100 μL) and incubated with anti-Fc depleted serum in a 96-deep well plate for two hours at room temperature. The paramagnetic beads were washed with 3×600 μL with HBS-SP, 1×600 μL of water, and 1×600 μL of 10% acetonitrile. Anti-hCD22 antibodies were eluted via incubation of the streptavidin beads with 70 μL of 1% formic acid in 30% acetonitrile/70% water for 15 minutes at room temperature. Each sample was then transferred to an Eppendorf tube and completely dried prior to LC-MS analysis.

### Example 4. Liquid Chromatography-Mass Spectrometry and Database Searching

### Example 4.1. Liquid Chromatography-Mass Spectrometry (LC-MS)

Anti-hFc and anti-hCD22 antibodies were each individually dissolved in 10 μL of 8M urea and 20 mM TCEP in 20 mM Tris-HCl (pH 8.0) at 37° C. for 1 hour. The denatured and reduced sample was then alkylated with 5 mM iodoacetamide for 30 min followed by overnight trypsin (w/v=1:20) digestion at 37° C. Tryptic peptides were analyzed by nano-LC1200 High Performance Liquid Chromatography coupled to a Q Exactive mass spectrometer. Peptides were first trapped onto a 75 μm×2 cm C18 trap column at a flow rate of 4 μL/min followed by separation at 250 nL/min using a 75 μm×25 cm C18 column at 40° C. with the following gradients: 5%-30% acetonitrile in 157 minutes; 30%-40% acetonitrile in 15 minutes; 40%-90% acetonitrile in 2 minutes, and 90% ACN for 15 min. Mass spectra were acquired under positive mode using following parameters: MS1 resolution: 70,000; MS1 target: 1E6; maximum injection time: 100 ms; scan range: 350 to 1,800 m/z; MS/MS resolution: 17,500; MS/MS target: 2e5; Top N: 10; isolation window: 2 Th; charge exclusion: 1, >5; dynamic exclusion: 30 sec.

### Example 4.2. Database Searching

The acquired LC-MS data from each immunized ULC mouse serum sample was searched against the corresponding database generated via NGS sequencing using Byonic™ search engine manufactured by Protein Metrics. The searching parameters were as follows: Cleavage site: lysine or Arginine; Cleavage site: C-terminal; Digestion specificity: fully specific; Missed cleavages: 2; Precursor mass tolerance: 10 ppm; Fragmentation type: HCD; Fragment mass tolerance: 20 ppm; Fixed modification: carbamidomethyl at cysteine. The top 200 hits were ranked based on sequence coverage and peptide confidence and checked manually.

### Example 5. Antibody Sequence Selection

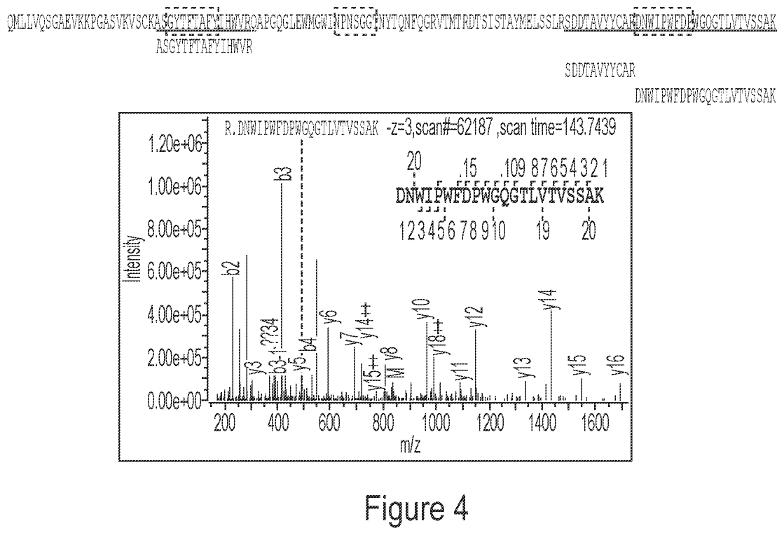

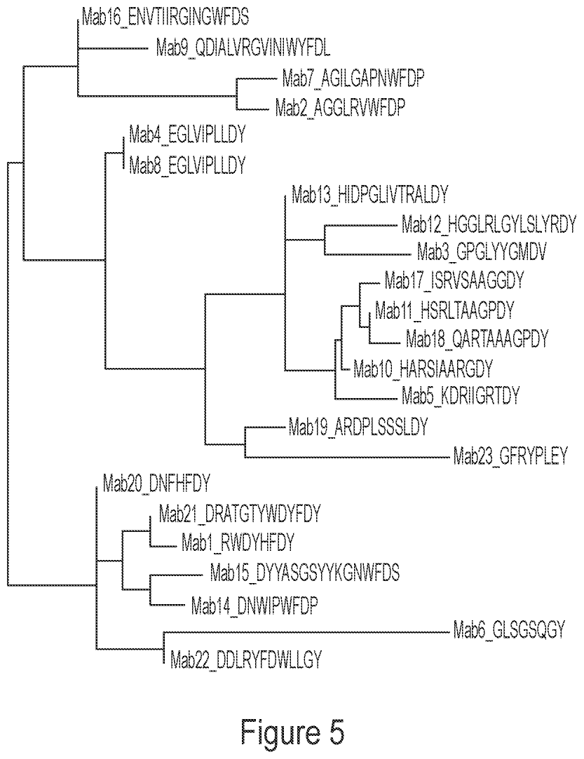

The top 200 sequence hits were manually checked for the spectra quality of all matched CDR3 peptides to make sure the majority of the fragment ions can be interpreted by the assigned peptide sequence. One or more unique CDR3 peptides with good spectra qualities were required for the antibody sequence to be a positive identification. Sequences were mapped into the CDR3 database and grouped based on CDR3. Antibodies were selected for cloning based on the following parameters: 1) exact match of unique CDR3 peptides; 2) exact match of unique CDR1 and CDR2 peptides; 3) exact match of unique framework peptides; 4) the number of next generation sequence counts; 5) excluding the CDR sequence with methionine and potential N glycosylation. Example of the selection of anti-CD22 antibody Bone629 (BM_629, mAb14) based on mass spectrometry spectra match and NGS from a group of anti-CD22 antibodies containing a homologous CDR3 sequence is shown in FIG. 4. The manual check resulted in a total of 50 antibodies for expression and cloning. To obtain a more diverse repertoire of antibody coverage for cloning, the sequences from universal light chain mice were grouped based on CDR3 homology. Twenty-three specific anti-CD22 antibodies representing diverse CDR3 groups are shown in FIG. 5.

### Example 6. Cloning and Transfection

Variable domain nucleotide sequences of hCD22 antibody candidates (n=23) were codon optimized for Chinese Hamster Ovary (CHO) cell expression and synthesized as gblocks (Integrated DNA Technologies). Variable domain gblocks were cloned into a vector in operable linkage with human immunoglobulin heavy chain constant region. Heavy chain vectors (1 μg) were paired with either light chain vector comprising ULC 3-20 in operable linkage with human immunoglobulin kappa light chain constant region (1 μg) or light chain vector comprising ULC 1-39 in operable linkage with human immunoglobulin kappa light chain constant region (1 μg) for transient transfection into a 9 cm² well of CHO K1 cells using Lipofectamine (Thermo Fisher Scientific). Supernatants (500 μL) were collected approximately 84 hours post transfection and concentrated, and the concentrate used for BIAcore binding analysis. Transfection efficiency was confirmed via western blotting under reducing conditions.

### Example 7. Kinetic Binding of Cloned Anti-CD22 Antibodies

### Example 7.1. Kinetic Binding Parameters for the Interaction of Cloned Anti-CD22 Antibodies with Human CD22

Supernatants from all transfected cells were analyzed for binding affinity and specificity against CD22 using SPR-Biacore technology. CD22 binding to each cloned antibody was measured at 25° C. and pH 7.4 by capturing the antibody from transfected CHO cell supernatant via its Fcγ domain to a goat anti-human Fcγ polyclonal antibody immobilized on a CM5 chip surface until a signal of approximately 165-202 relative units (RU) was reached, followed by injections of CD22 proteins. Recombinant CD22, at concentrations ranging from 0.313 nM to 10.0 nM, and a negative control at concentrations ranging from 1.25 nM to 40.0 nM, were individually injected over the surface captured anti-CD22 and a reference surface (anti-Fcγ-coupled chip surface without captured anti-CD22) for 3 minutes at a flow rate of 50 μL/min followed by a 10-minute (CD22) dissociation phase, and binding signal changes recorded. Regeneration of the chip was achieved using a 40 sec pulse of 10 mM glycine-HCl pH 1.5.

Kinetic binding parameters were determined from specific SPR-Biacore kinetic sensorgrams using a double referencing procedure. Double referencing was achieved by subtracting the signal for CD22 injected over the reference surface (goat anti-human Fcγ coupled surface only) from the signal for CD22 injected over the experimental surface (Fcγ captured anti-CD22 surface), thereby removing contributions from refractive index changes. In addition, the difference in signal changes resulting from the dissociation of captured anti-CD22 from the goat anti human Fcγ polyclonal antibody control buffer injections (no CD22) were also accounted for when calculating kinetic binding parameters.

The calculated kinetic binding parameters are summarized in Table 4.

TABLE 4
Summary of Kinetic Binding Parameters for selected anti-CD22 monoclonal antibodies with human CD22

| | | | | 90 nM | | | | |
|---|---|---|---|---|---|---|---|---|
| | | | Supe | hCD22.mmh | | | | |
| | Mouse | | Capture | Bound | ka | kd | KD | t½ |
| Sample | Type | NGS NO. | (RU) | (RU) | (1/Ms) | (1/s) | (M) | (min) |
| mAb1 | ULC 3-20 | BM_2841 | 193 | 107 | 3.79E + 05 | 2.13E − | 5.64E − | 5.4 |
| | | | | | | 03 | 09 | |
| mAb2 | ULC 3-20 | BM_7637 | 348 | 69 | 1.24E + 05 | 7.66E − | 6.19E − | 15.1 |
| | | | | | | 04 | 09 | |
| mAb3 | ULC 3-20 | BM_1224 | 176 | 20 | 6.28E + 04 | 8.73E − | 1.39E − | 13.2 |
| | | | | | | 04 | 08 | |
| mAb4 | ULC 3-20 | Spleen_583 | 161 | 53 | 1.96E + 05 | 7.85E − | 4.00E − | 1.5 |
| | | | | | | 03 | 08 | |
| mAb5 | ULC 3-20 | BM_2883 | 217 | 10 | 1.76E + 06 | 9.50E − | 5.39E − | 0.1 |
| | | | | | | 02 | 08 | |
| mAb6 | ULC 3-20 | BM_3347 | 255 | 40 | 6.98E + 04 | 6.94E − | 9.95E − | 1.7 |
| | | | | | | 03 | 08 | |
| mAb7 | ULC 3-20 | BM_50146 | 216 | 17 | 1.80E + 05 | 1.84E − | 1.02E − | 0.6 |
| | | | | | | 02 | 07 | |
| mAb8 | ULC 3-20 | Spleen_583 | 289 | 25 | 7.11E + 04 | 8.00E − | 1.12E − | 1.4 |
| | | | | | | 03 | 07 | |
| mAb9 | ULC 1-39 | Spleen_1170 | 151 | 18 | 6.39E + 06 | ≤1.00E − | 1.56E − | ≥115.5 |
| | | | | | | 04 | 11 | |
| mAb10 | ULC 1-39 | BM_11 | 295 | 16 | 6.78E + 04 | ≤1.00E − | 1.47E − | ≥115.5 |
| | | | | | | -04 | 09 | |
| mAb11 | ULC 1-39 | BM_314 | 409 | 30 | 5.75E + 04 | ≤1.00E − | 1.74E − | ≥115.5 |
| | | | | | | 04 | 09 | |
| mAb12 | ULC 1-39 | BM_2090 | 403 | 53 | 5.33E + 04 | ≤1.00E − | 1.87E − | ≥115.5 |
| | | | | | | 04 | 09 | |
| mAb13 | ULC 1-39 | Spleen_598 | 278 | 34 | 3.82E + 04 | ≤1.00E − | 2.62E − | ≥115.5 |
| | | | | | | 04 | 09 | |
| mAb14 | ULC 1-39 | BM_629 | 196 | 21 | 2.43E + 04 | ≤1.00E − | 4.12E − | ≥115.5 |
| | | | | | | 04 | 09 | |
| mAb15 | ULC 1-39 | BM_32414 | 319 | 59 | 9.67E + 04 | 6.90E − | 7.13E − | 16.8 |
| | | | | | | 04 | 09 | |
| mAb16 | ULC 1-39 | Spleen_39 | 196 | 63 | 1.59E + 05 | 1.42E − | 8.96E − | 8.1 |
| | | | | | | 03 | 09 | |
| mAb17 | ULC 1-39 | BM_789 | 339 | 32 | 5.51E + 04 | 5.41E − | 9.82E − | 21.4 |
| | | | | | | 04 | 09 | |
| mAb18 | ULC 1-39 | BM_435 | 325 | 63 | 8.66E + 04 | 1.18E − | 1.37E − | 9.8 |
| | | | | | | 03 | 08 | |
| mAb19 | ULC 1-39 | BM_1083 | 310 | 56 | 1.62E + 05 | 6.69E − | 4.13E − | 1.7 |
| | | | | | | 03 | 08 | |
| mAb20 | ULC 1-39 | BM_511 | 272 | 48 | 1.22E + 05 | 6.95E − | 5.70E − | 1.7 |
| | | | | | | 03 | 08 | |
| mAb21 | ULC 1-39 | BM_27845 | 325 | 26 | 1.85E + 05 | 2.11E − | 1.14E − | 0.5 |
| | | | | | | 02 | 07 | |
| mAb22 | ULC 1-39 | BM_316 | 210 | 12 | 4.96E + 05 | 5.68E − | 1.15E − | 0.2 |
| | | | | | | 02 | 07 | |
| mAb23 | ULC 1-39 | BM_3615 | 469 | 25 | 7.32E + 04 | 1.17E − | 1.60E − | 1.0 |
| | | | | | | 02 | 07 | |

As evident from the data in Table 4 above, of the 23 supernatants analyzed for binding CD22, a number showed high affinity against human CD22, with K_{D} of less than 1.0×10⁻⁸M. From these, 11 were submitted for antibody purification. All 11 purified antibodies showed specific binding to human CD22 but not mouse CD22 (data not shown).

An additional 16 monoclonal antibody sequences were chosen for BiaCore analysis based solely on sequence homology of heavy chain variable domains to anti-CD22 mAB BM_629 to heavy chain variable domains. All 16 monoclonal antibodies showed significantly reduced or lost binding properties against CD22 (Table 5), suggesting that LC-MS spectra provides essential information in antibody selection.

TABLE 5
Summary of Kinetic Binding Parameters for selected anti-CD22 antibodies based solely on sequence homology.

| | | | 90 nM | | | | |
|---|---|---|---|---|---|---|---|
| | | Supe | hCD22.mmh | | | | |
| | | Capture | Bound | ka | kd | KD | t½ |
| mAb | Description | (RU) | (RU) | (1/Ms) | (1/s) | (M) | (min) |
| mAb14 | BM_629 | 187 | 46 | 5.01E + 04 | 3.52E − 04 | 7.03E − 09 | 32.8 |
| mAb14_1 | BM_22525 | 103 | 0 | NB | NB | NB | NB |
| mAb14_2 | BM_8760 | 94 | −3 | NB | NB | NB | NB |
| mAb14_3 | BM_19611 | 199 | 2 | NB | NB | NB | NB |
| mAb14_4 | BM_58661 | 126 | −5 | NB | NB | NB | NB |
| mAb14_5 | BM_82128 | 76 | −5 | NB | NB | NB | NB |
| mAb14_6 | BM_20548 | 49 | −2 | NB | NB | NB | NB |
| mAb14_7 | BM_50339 | 45 | −5 | NB | NB | NB | NB |
| mAb14_8 | BM_51082 | 61 | −4 | NB | NB | NB | NB |
| mAb14_9 | BM_60395 | 252 | 39 | 4.06E + 04 | 3.50E − 03 | 8.63E − 08 | 3.3 |
| mAb14_10 | BM_63775 | 73 | −7 | NB | NB | NB | NB |
| mAb14_11 | BM_6421 | 78 | −4 | NB | NB | NB | NB |
| mAb14_12 | BM_72341 | 366 | 28 | 3.71E + 04 | 1.03E − 02 | 2.79E − 07 | 1.1 |
| mAb14_13 | BM_9387 | 62 | −6 | NB | NB | NB | NB |
| mAb14_14 | BM_53145 | 61 | −6 | NB | NB | NB | NB |
| mAb14_15 | BM_50411 | 100 | −4 | NB | NB | NB | NB |
| mAb14_16 | BM_43396 | 481 | −5 | NB | NB | NB | NB |

Thus, the exemplary methods described herein are able to identify antibody variable domain sequences from particular in vivo sources of antibody within an immunized host (e.g., serum) with desired characteristics. The provided methods provides a robust means for quickly identifying antibodies for antibodies with desired characteristics (e.g., high binding affinity) from a genetically modified non-human animal (e.g., rodent, e.g., mouse).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of identifying a human immunoglobulin variable domain or CDR sequence of an antibody specific for an antigen, comprising:
obtaining a plurality of peptide sequences of human immunoglobulin heavy chain and/or light chain variable domains that were obtained from a sample comprising a population of antibodies produced by a rodent immunized with the antigen, and
interrogating a library of human immunoglobulin heavy chain and/or light chain variable domain sequences with the plurality of peptide sequences, wherein the library comprises a plurality of human immunoglobulin heavy chain and/or light chain variable domain sequences encoded by B cells of the immunized rodent, thereby obtaining a human immunoglobulin variable domain or CDR sequence of an antibody specific for the antigen, and
wherein the immunized rodent comprises in its germline genome:
an immunoglobulin heavy chain variable region comprising one or more human heavy chain V gene segments, one or more human D gene segments, and one or more human heavy chain J gene segments, wherein the heavy chain variable region is operably linked to a constant region, and
an immunoglobulin light chain variable region comprising one or more human light chain V gene segments and one or more human light chain J gene segments, wherein the light chain is operably linked to a constant region.

2. A method of identifying a human immunoglobulin variable domain or CDR sequence of an antibody specific for an antigen, comprising:
obtaining a library of human immunoglobulin heavy chain and/or light chain variable domain sequences comprising a plurality of human immunoglobulin heavy chain and/or light chain variable domain sequences encoded by B cells of a rodent immunized with the antigen,
interrogating the library with a plurality of peptide sequences of human immunoglobulin heavy chain and/or light chain variable domains that were obtained from a sample comprising a population of antibodies produced by the rodent immunized with the antigen, and
wherein the immunized rodent comprises in its germline genome:
an immunoglobulin heavy chain variable region comprising one or more human heavy chain V gene segments, one or more human D gene segments, and one or more human heavy chain J gene segments, wherein the heavy chain variable region is operably linked to a murine constant region, and
an immunoglobulin light chain variable region comprising one or more human light chain V gene segments and one or more human light chain J gene segments, wherein the light chain is operably linked to a murine constant region.

3. The method of claim 1, wherein the plurality of human immunoglobulin heavy chain and/or light chain variable domain sequences of the library were obtained from sequencing a sample comprising a population of B cells from bone marrow and/or spleen of the rodent.

4. The method of claim 1, wherein the plurality of human immunoglobulin heavy chain and/or light chain variable domain sequences of the library were obtained from sequencing of cDNA comprising rearranged heavy chain VDJ sequences and/or rearranged light chain VJ sequences.

5. The method of claim 4, wherein the sequencing is by next generation DNA sequencing.

6. The method of claim 1, wherein the sample comprising a population of antibodies produced by the rodent immunized with the antigen is derived from serum, plasma, lymphoid organs, gut, cerebrospinal fluid, brain, spinal cord, and/or placenta of the rodent.

7. The method of claim 1, wherein the plurality of peptide sequences of human immunoglobulin heavy chain and/or light chain variable domains were obtained or determined by mass spectrometry (MS).

8. The method of claim 7, wherein the plurality of peptide sequences of human immunoglobulin heavy chain and/or light chain variable domains were obtained or determined by combined liquid chromatography and mass spectrometry (LC-MS).

9. The method of claim 7, wherein the sample comprising a population of antibodies produced by the rodent immunized with the antigen was denatured prior to MS analysis.

10. The method of claim 7, wherein the sample comprising a population of antibodies produced by the rodent immunized with the antigen was proteolytically digested prior to MS analysis.

11. The method of claim 7, wherein the sample comprising a population of antibodies produced by the rodent immunized with the antigen was enriched for one or more characteristics prior to MS analysis.

12. The method of claim 11, wherein the sample comprising a population of antibodies produced by the rodent immunized with the antigen was enriched for antibodies that bind the antigen.

13. The method of claim 12, wherein the sample comprising a population of antibodies produced by the rodent immunized with the antigen was depleted for antibodies that bind a second, different antigen.

14. The method of claim 1, wherein interrogating the library of human immunoglobulin heavy chain and/or light chain variable domain sequences with the plurality of peptide sequences comprises aligning the peptide sequences to each other and to the amino acid sequences of the plurality of human immunoglobulin heavy chain and/or light chain variable domains.

15. The method of claim 7, wherein the library is a library of human immunoglobulin heavy chain variable domain sequences and the interrogating with the plurality of peptide sequences is based on one or more of:
(1) a match of a CDR3 sequence in the library of human immunoglobulin heavy chain and/or light chain variable domain sequences to a unique peptide obtained or determined by MS,
(2) a match of unique CDR1 and/or CDR2 sequences in the library of human immunoglobulin heavy chain and/or light chain variable domain sequences to one or more unique peptides obtained or determined by MS,
(3) a match of one or more framework sequences in the library of human immunoglobulin heavy chain and/or light chain variable domain sequences to one or more unique peptides obtained or determined by MS,
(4) a number of next generation sequencing counts for a sequence in the library of human immunoglobulin heavy chain and/or light chain variable domain sequences,
(5) exclusion of CDR sequences with methionine, and
(6) exclusion of CDR sequences with potential N glycosylation.

16. The method of claim 1, wherein interrogating the library identifies a plurality of human immunoglobulin variable domain or CDR sequences of antibodies specific for the antigen, and wherein the plurality of human immunoglobulin variable domain or CDR sequences are ranked.

17. The method of claim 1, wherein the rodent is a rat.

18. The method of claim 1, wherein the rodent is a mouse.

19. The method of claim 1, wherein the immunoglobulin heavy chain variable region is operably linked to a mouse heavy chain constant region, and/or the immunoglobulin light chain variable region is operably linked to a mouse light chain constant region.

20. The method of claim 19, wherein the immunoglobulin heavy chain variable region operably linked to a mouse heavy chain constant region is at the endogenous mouse heavy chain locus, and/or the immunoglobulin light chain variable region operably linked to a mouse light chain constant region is at the endogenous mouse light chain locus.

21. The method of claim 1, wherein the immunoglobulin heavy chain variable region comprises a plurality of human heavy chain V gene segments, a plurality of human D gene segments, and a plurality of human heavy chain J gene segments, wherein the heavy chain variable region is operably linked to a murine heavy chain constant region, and
the immunoglobulin light chain variable region comprises:
(i) a universal light chain encoding sequence comprising a rearranged human light chain variable region comprising a single human V_{L} gene segment and single human light κ_{L} gene segment, operably linked to a mouse light chain constant region;
(ii) a restricted light chain variable region, comprising two unrearranged human V_{L} gene segments and one or more unrearranged human J_{L} gene segments, operably linked to a mouse light chain constant region; or
(iii) a histidine modified light chain variable region comprising one or more human light chain V gene segments and one or more human light chain J gene segments, further comprising substitution or insertion of at least one histidine for a non-histidine residue, operably linked to a mouse light chain constant region.

22. The method of claim 1, wherein the immunoglobulin light chain variable region comprises a plurality of human light chain V gene segments and a plurality of human light chain J gene segments, wherein the light chain variable region is operably linked to a murine light chain constant region, and wherein the immunoglobulin heavy chain variable region comprises:
(i) a restricted unrearranged heavy chain variable region, comprising a single human V_{H} gene segment, one or more unrearranged human D_{H} gene segments, and one or more unrearranged human J_{H} gene segments, operably linked to a mouse heavy chain constant region;
(ii) a universal heavy chain encoding sequence comprising a single rearranged human heavy chain variable region comprising a single human V_{H} gene segment, a single human D_{H} gene segment, and a single human J_{H} gene segment, operably linked to a mouse heavy chain constant region;
(iii) a histidine modified unrearranged heavy chain variable region, comprising one or more unrearranged human V_{H} gene segments, one or more unrearranged human D_{H} gene segments, and one or more unrearranged human J_{H} gene segments, further comprising substitution or insertion of at least one histidine for a non-histidine residue, operably linked to a mouse heavy chain constant region.

23. The method of claim 1, wherein the immunoglobulin light chain variable region comprises a universal light chain encoding sequence comprising a rearranged human light chain variable region comprising a single human Vκ gene segment and single human light J_{κ} gene segment, wherein the rearranged human light chain variable region is at the endogenous mouse κ light chain locus and operably linked to a mouse light chain constant region, and wherein the immunoglobulin heavy chain variable region comprises a plurality of human heavy chain V gene segments, a plurality of human D gene segments, and a plurality of human heavy chain J gene segments, wherein the heavy chain variable region is operably linked to a murine heavy chain constant region.

24. The method of claim 1, wherein the immunoglobulin light chain variable region comprises an engineered immunoglobulin κ light chain locus that comprises a single rearranged human immunoglobulin λ light chain variable region comprising a human Vλ gene segment joined to a human Jλ gene segment, and wherein the immunoglobulin heavy chain variable region comprises a plurality of human heavy chain V gene segments, a plurality of human D gene segments, and a plurality of human heavy chain J gene segments, wherein the heavy chain variable region is operably linked to a murine heavy chain constant region.

25. The method of claim 1, wherein the genetically modified mouse further comprises a functional ADAM6 gene, optionally wherein the functional ADAM6 gene is a mouse ADAM6 gene.

26. The method of claim 1, wherein the genetically modified mouse further expresses an exogenous terminal deoxynucleotidyl transferase (TdT) gene.

27. The method of claim 1, wherein the method further comprises expressing a nucleotide sequence encoding the identified human immunoglobulin heavy chain and/or light chain variable domain in a recombinant antigen-binding protein.

28. The method of claim 27, wherein the recombinant antigen-binding protein is a human antibody.

29. The method of claim 27, wherein the recombinant antigen-binding protein is a bispecific antibody.

30. A method for making an antibody comprising:
(a) expressing in a host cell (i) a nucleic acid encoding an immunoglobulin heavy chain comprising a human immunoglobulin heavy chain variable region sequence operably linked to an immunoglobulin heavy chain constant region sequence and (ii) a nucleic acid encoding an immunoglobulin light chain comprising a human immunoglobulin light chain variable region sequence operably linked to an immunoglobulin light chain constant region sequence, wherein the human immunoglobulin heavy chain variable region sequence and/or the human immunoglobulin light chain variable region sequence encode human immunoglobulin heavy chain variable domain and/or human immunoglobulin light chain variable domain, respectively, that were identified by a method of claim 1; and
(b) culturing the host cell under conditions such that the host cell expresses an antibody comprising the immunoglobulin heavy chain and the immunoglobulin light chain.

31. A method of making a fully human immunoglobulin heavy chain and/or fully human immunoglobulin light chain comprising:
(a) identifying a human immunoglobulin heavy chain and/or light chain variable domain sequence by a method of claim 1;
(b) operably linking the nucleic acid encoding the human immunoglobulin heavy chain variable domain with a nucleic acid encoding a human immunoglobulin heavy chain constant domain to form a fully human immunoglobulin heavy chain and/or operably linking the nucleic acid encoding the human immunoglobulin light chain variable domain with a nucleic acid encoding a human immunoglobulin light chain constant domain to form a fully human immunoglobulin light chain; and
(c) expressing the fully human immunoglobulin heavy chain and/or fully human immunoglobulin light chain.


SEQUENCE LISTING (40 sequences)

SEQ ID NO: 1 - DNA, 26 bp, Artificial Sequence
  Description of Artificial Sequence Synthetic oligonucleotide
  Description of Combined DNA/RNA Molecule Synthetic oligonucleotide
  Template Switching Primer
caccatcgatgtcgacacgcctaggg

SEQ ID NO: 2 - DNA, 23 bp, Artificial Sequence
  Description of Artificial Sequence Synthetic primer
  1st Round PCR Primer - IgG Constant
ggaaggtgtgcacaccgctggac

SEQ ID NO: 3 - DNA, 23 bp, Artificial Sequence
  Description of Artificial Sequence Synthetic primer
  1st Round PCR Primer - IgG Constant
ggaaggtgtgcacactgctggac

SEQ ID NO: 4 - DNA, 23 bp, Artificial Sequence
  Description of Artificial Sequence Synthetic primer
  1st Round PCR Primer - IgG Constant
ggaaggtgtgcacaccactggac

SEQ ID NO: 5 - DNA, 23 bp, Artificial Sequence
  Description of Artificial Sequence Synthetic primer
  1st Round PCR Primer - IgG Constant
agactgtgcgcacaccgctggac

SEQ ID NO: 6 - DNA, 27 bp, Artificial Sequence
  Description of Artificial Sequence Synthetic primer
  1st Round PCR Primer - TS Specific
aagcagtggtatcaacgcagagtacat

SEQ ID NO: 7 - DNA, 55 bp, Artificial Sequence
  Description of Artificial Sequence Synthetic primer
  2nd Round PCR Primer - IgG Constant
acactctttccctacacgacgctcttccgatctagtggatagacagatgggggtg

SEQ ID NO: 8 - DNA, 55 bp, Artificial Sequence
  Description of Artificial Sequence Synthetic primer
  2nd Round PCR Primer - IgG Constant
acactctttccctacacgacgctcttccgatctagtggatagactgatgggggtg

SEQ ID NO: 9 - DNA, 55 bp, Artificial Sequence
  Description of Artificial Sequence Synthetic primer
  2nd Round PCR Primer - IgG Constant
acactctttccctacacgacgctcttccgatctagtggatagaccgatggggctg

SEQ ID NO: 10 - DNA, 55 bp, Artificial Sequence
  Description of Artificial Sequence Synthetic primer
  2nd Round PCR Primer - IgG Constant
acactctttccctacacgacgctcttccgatctaagggatagacagatggggctg

SEQ ID NO: 11 - DNA, 57 bp, Artificial Sequence
  Description of Artificial Sequence Synthetic primer
  2nd Round PCR Primer - TS Specific
gtgactggagttcagacgtgtgctcttccgatctcaccatcgatgtcgacacgccta

SEQ ID NO: 12 - DNA, 68 bp, Artificial Sequence
  Description of Artificial Sequence Synthetic primer
  Final Round PCR Primer - Forward
  modified_base (30)..(35): a, c, t, g, unknown or other
  misc_feature (30)..(35): Six base pair index sequence to enable multiplexing samples for sequencing
aatgatacggcgaccaccgagatctacacnnnnnnacactctttccctacacgacgctcttccgatct

SEQ ID NO: 13 - DNA, 64 bp, Artificial Sequence
  Description of Artificial Sequence Synthetic Primer
  Final Round PCR Primers - Reverse
  modified_base (25)..(30): a, c, t, g, unknown or other
  misc_feature (25)..(30): Six base pair index sequence to enable multiplexing samples for sequencing
caagcagaagacggcatacgagatnnnnnngtgactggagttcagacgtgtgctcttccgatct

SEQ ID NO: 14 - protein, 120 aa, Artificial Sequence
  Description of Artificial Sequence Synthetic polypeptide
QMLLVQSGAEVKKPGASVKVSCKASGYTFTAFYIHWVRQAPGQGLEWMGWINPNSGGTNYTQNFQGRVTMTRDTSISTAYMELSSLRSDDTAVYYCARDNWIPWFDPWGQGTLVTVSSAK

SEQ ID NO: 15 - protein, 15 aa, Artificial Sequence
  Description of Artificial Sequence Synthetic peptide
ASGYTFTAFYIHWVR

SEQ ID NO: 16 - protein, 11 aa, Artificial Sequence
  Description of Artificial Sequence Synthetic peptide
SDDTAVYYCAR

SEQ ID NO: 17 - protein, 22 aa, Artificial Sequence
  Description of Artificial Sequence Synthetic peptide
DNWIPWFDPWGQGTLVTVSSAK

SEQ ID NO: 18 - protein, 23 aa, Artificial Sequence
  Description of Artificial Sequence Synthetic peptide
RDNWIPWFDPWGQGTLVTVSSAK

SEQ ID NO: 19 - protein, 15 aa, Artificial Sequence
  Description of Artificial Sequence Synthetic peptide
ENVTIIRGINGWFDS

SEQ ID NO: 20 - protein, 17 aa, Artificial Sequence
  Description of Artificial Sequence Synthetic peptide
QDIALVRGVINIWYFDL

SEQ ID NO: 21 - protein, 12 aa, Artificial Sequence
  Description of Artificial Sequence Synthetic peptide
AGILGAPNWFDP

SEQ ID NO: 22 - protein, 10 aa, Artificial Sequence
  Description of Artificial Sequence Synthetic peptide
AGGLRVWFDP

SEQ ID NO: 23 - protein, 10 aa, Artificial Sequence
  Description of Artificial Sequence Synthetic peptide
EGLVIPLLDY

SEQ ID NO: 24 - protein, 14 aa, Artificial Sequence
  Description of Artificial Sequence Synthetic peptide
HIDPGLIVTRALDY

SEQ ID NO: 25 - protein, 15 aa, Artificial Sequence
  Description of Artificial Sequence Synthetic peptide
HGGLRLGYLSLYRDY

SEQ ID NO: 26 - protein, 10 aa, Artificial Sequence
  Description of Artificial Sequence Synthetic peptide
GPGLYYGMDV

SEQ ID NO: 27 - protein, 11 aa, Artificial Sequence
  Description of Artificial Sequence Synthetic peptide
ISRVSAAGGDY

SEQ ID NO: 28 - protein, 11 aa, Artificial Sequence
  Description of Artificial Sequence Synthetic peptide
HSRLTAAGPDY

SEQ ID NO: 29 - protein, 11 aa, Artificial Sequence
  Description of Artificial Sequence Synthetic peptide
QARTAAAGPDY

SEQ ID NO: 30 - protein, 11 aa, Artificial Sequence
  Description of Artificial Sequence Synthetic peptide
HARSIAARGDY

SEQ ID NO: 31 - protein, 9 aa, Artificial Sequence
  Description of Artificial Sequence Synthetic peptide
KDRIIGTDY

SEQ ID NO: 32 - protein, 11 aa, Artificial Sequence
  Description of Artificial Sequence Synthetic peptide
ARDPLSSSLDY

SEQ ID NO: 33 - protein, 8 aa, Artificial Sequence
  Description of Artificial Sequence Synthetic peptide
GFRYPLEY

SEQ ID NO: 34 - protein, 7 aa, Artificial Sequence
  Description of Artificial Sequence Synthetic peptide
DNFHFDY

SEQ ID NO: 35 - protein, 13 aa, Artificial Sequence
  Description of Artificial Sequence Synthetic peptide
DRATGTYWDYFDY

SEQ ID NO: 36 - protein, 8 aa, Artificial Sequence
  Description of Artificial Sequence Synthetic peptide
RWDYHFDY

SEQ ID NO: 37 - protein, 16 aa, Artificial Sequence
  Description of Artificial Sequence Synthetic peptide
DYYASGSYYKGNWFDS

SEQ ID NO: 38 - protein, 9 aa, Artificial Sequence
  Description of Artificial Sequence Synthetic peptide
DNWIPWFDP

SEQ ID NO: 39 - protein, 8 aa, Artificial Sequence
  Description of Artificial Sequence Synthetic peptide
GLSGSQGY

SEQ ID NO: 40 - protein, 12 aa, Artificial Sequence
  Description of Artificial Sequence Synthetic peptide
DDLRYFDWLLGY